SEQUENCE LISTING (8 sequences)

SEQ ID NO: 1 - DNA, 11 bp, Artificial Sequence
  partial plasmid pSNR52 backbone sequence
tgatcgagacc

SEQ ID NO: 2 - DNA, 11 bp, Artificial Sequence
  partial plasmid pSNR52 backbone sequence
ggtctctgttt

SEQ ID NO: 3 - DNA, 12 bp, Artificial Sequence
  partial DNA insert sequence
cggtctcatgat

SEQ ID NO: 4 - DNA, 12 bp, Artificial Sequence
  partial DNA insert sequence
gggcttgagacc

SEQ ID NO: 5 - DNA, 12 bp, Artificial Sequence
  partial DNA insert sequence
gggtctcaggct

SEQ ID NO: 6 - DNA, 12 bp, Artificial Sequence
  partial DNA insert sequence
gcgcatgagacc

SEQ ID NO: 7 - DNA, 11 bp, Artificial Sequence
  partial DNA insert sequence
ggtctcacgca

SEQ ID NO: 8 - DNA, 12 bp, Artificial Sequence
  partial DNA insert sequence
agtttagagacc

(19) United States
(12) Patent Application Publication
(10) Pub. No.: US 2023/0265460 A1
(43) Pub. Date: Aug. 24, 2023

(54) A MODULAR AND POOLED APPROACH FOR MULTIPLEXED CRISPR GENOME EDITING

(71) Applicant: Zymergen Inc., Emeryville, CA (US)

(72) Inventors: Solomon Henry STONEBLOOM, Alameda, CA (US); Colin Scott MAXWELL, Oakland, CA (US); Shawn SZYJKA, Martinez, CA (US)

(21) Appl. No.: 18/017,956

(22) PCT Filed: Jul. 9, 2021
(86) PCT No.: PCT/US2021/041095

§ 371 Date: Jan. 25, 2023

Related U.S. Application Data
(60) Provisional application No. 63/057,096, filed on Jul. 27, 2020.

(51) Int. Cl. C12N 15/90; C12N 15/11; C12N 9/22

(52) U.S. Cl. CPC C12N 15/905; C12N 15/11; C12N 9/22; C12N 2310/20; C12N 2800/80

(57) ABSTRACT

Provided herein are nucleic acid constructs comprising multiple guide RNAs interspersed with tRNA sequence at regular intervals as well as expression vectors and compositions comprising the same. Also provided herein are methods for assembling the nucleic acid constructs comprising multiple guide RNAs interspersed with tRNA sequence at regular intervals in a pooled and/or modular manner. Methods for using the nucleic acid constructs comprising multiple guide RNAs interspersed with tRNA sequence at regular intervals to facilitate multiplexed genomic editing of a host cell comprising said nucleic acid constructs are also provided herein.

### CROSS-REFERENCE TO RELATED APPLICATIONS

This application claims the benefit of priority to U.S. Provisional Application Ser. No. 63/057,096, filed Jul. 27, 2020, which is herein incorporated by reference in its entirety for all purposes.

### FIELD

The present disclosure is directed to constructs, compositions and methods for the modular assembly of plasmids comprising multiple guide-RNA (gRNA) sequences separated by tRNA sequences. The assembled plasmids can subsequently be used for multiplexed genetic modification of recipient cells using CRISPR-mediated homology-directed repair (HDR).

### STATEMENT REGARDING SEQUENCE LISTING

The Sequence Listing associated with this application is provided in text format in lieu of a paper copy and is hereby incorporated by reference into the specification. The name of the text file containing the Sequence Listing is ZYMR_056_01WO_SeqList_ST25.txt. The text file is ˜2 KB, was created on Jun. 14, 2021, and is being submitted electronically via EFS-Web.

### BACKGROUND

The CRISPR-Cas9 programmable RNA genome editing method has been used in a myriad of organisms including plant cells and yeast strains (see Lowder, L. G., et al., (2015). Plant Physiol. 169: 971-985; DiCarlo, J. E., et al. (2013) Nucleic Acids Res. 41(7):4336-43 and Ryan, O. W., et al. (2014) eLife 3: e03703). In order to try to achieve multiplexed editing of host cell genomes using the CRISPR/Cas9 system, several different strategies have been tried. For example, one strategy entails introducing a single plasmid containing multiple independent gRNA expression cassettes and a Cas9 gene in order to facilitate CRISPR/Cas9 genomic editing across multiple genomic loci. While this strategy has shown some utility in both plants (Lowder, L. G., et al., (2015) Plant Physiol. 169: 971-985) and yeast (Ryan, O. W., et al. (2014) eLife 3:e03703), the use of multiple independent expression cassettes on the single plasmid can be complicated to implement, and may also contribute to plasmid instability. Also, this type of single plasmid, multiple individual expression cassette system requires, at a minimum, that independent promoters and terminators be used for each gRNA being expressed, which can lead to variable expression levels of the individual gRNAs, and, as a result, may sometimes requires the use of additional genetic elements to aid in expression of each gRNA (see Ryan, O. W., et al. (2014) eLife 3: e03703). Alternatively, a strategy for facilitating multiplex CRISPR/Cas9 genomic editing utilized in recipient cells ranging from plants (Xie et al, PNAS 2015 Mar. 17; 112(11):3570-5) to yeast (Zhang et al, (2019). Nature Communications, 10(1), 1-10) to human cell lines (Dong et al., Biochem Biophys Res Commun 2017 Jan. 22; 482(4): 889-895) has recently been reported that consists of building polycistronic arrays of gRNAs in a single plasmid with each gRNA being separated by tRNAs. Following introduction of these polycistronic tRNA-gRNA arrays into the recipient cells, the endogenous tRNA processing system of said host cell then processes the tRNA-gRNA arrays into multiple individual gRNAs that can be used for genetic editing at multiple genomic loci upon expression of Cas9 in the recipient cells. While this system has shown utility for multiplexed genetic editing in the recipient cells tested, the assembly of the gRNAs arrays in this system was accomplished with Golden Gate cloning using ligation sites within the variable spacer sequences, thereby limiting efficient modularity of the system since use of the spacers themselves as the ligation sites for assembly means that each PCR amplicon used in the assembly of the multi-gRNA cassette can only be used to produce a single assembly of gRNAs in a particular order. Further, the amount of repeated DNA sequence in this system due to the use of a single tRNA across the construct can lead to unwanted construct instability (see Oliveira et al., Appl Microbiol Biotechnol 87, 2157-2167 (2010)), thereby decreasing the potential efficiency of genome editing.

Thus, there is a need in the art for constructs, compositions and methods for the simple, efficient assembly of multiple gRNA containing constructs that is modular in nature and effective for use in genetic editing of recipient cell at one or across multiple genomic loci using the CRISPR system. The constructs, compositions and methods provided herein address this need.

### SUMMARY

In one aspect, provided herein is a nucleic acid construct comprising, from 5′ to 3′, a first type IIs restriction site, a guide RNA (gRNA) and a tRNA sequence with a second type IIs restriction site within the tRNA sequence, wherein the gRNA comprises a spacer sequence comprising sequence complementary to a target sequence present at a locus in a genetic element in a cell. In some cases, the tRNA sequence comprises one or more of the group consisting of a pretRNA acceptor stem, a D-loop arm and a TψC-loop arm. In some cases, the tRNA sequence comprises an active site for one or more of the group consisting of RNase P, RNase Z, RNase E, RNase F and RNase D. In some cases, the tRNA sequence comprises an entire pre-tRNA sequence. In some cases, the pre-tRNA sequence is selected from tRNA-ser, tRNA-gln, tRNA-lys or tRNA-gly. In some cases, the second type IIs restriction site is within the 3′ terminus of the tRNA sequence. In some cases, the first and/or second type IIs restriction site is BsaI. In some cases, the gRNA is a single guide RNA (sgRNA). In some cases, the cell is a prokaryotic cell or eukaryotic cell.

In another aspect, provided herein is a composition comprising: (a) a plasmid backbone comprising, from 5′ to 3′, a promoter sequence, a first type IIs restriction site, a stuffer sequence and a second type IIs restriction site; and (b) a plurality of gRNA-tRNA sequence parts, wherein each gRNA-tRNA sequence part in the plurality, comprises, from 5′ to 3′, a first type IIs restriction site, a guide RNA (gRNA) and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality, wherein the first and second type IIs restriction sites in the plasmid backbone allow for insertion of each gRNA-tRNA sequence part of the plurality of gRNA-tRNA sequence parts of (b) into the plasmid backbone upon cleavage. In some cases, the promoter sequence is a Pol III promoter. In some cases, the promoter sequence is a pSNR52 promoter. In some cases, the second type IIs restriction site in each gRNA-tRNA sequence part from the plurality is within the 3′ terminus of the tRNA sequence. In some cases, the tRNA sequence in each gRNA-tRNA sequence part from the plurality comprises one or more of the group consisting of a pretRNA acceptor stem, a D-loop arm and a TψC-loop arm. In some cases, the tRNA sequence in each gRNA-tRNA sequence part from the plurality comprises an active site for one or more of the group consisting of RNase P, RNase Z, RNase E, RNase F and RNase D. In some cases, the tRNA sequence in each gRNA-tRNA sequence part from the plurality comprises a full pre-tRNA sequence. In some cases, the pre-tRNA sequence is selected from tRNA-ser, tRNA-gln, tRNA-lys or tRNA-gly. In some cases, the composition further comprises a type IIs restriction enzyme that recognizes the first and/or the second type IIs restriction sites in the plasmid backbone and each gRNA-tRNA sequence part from the plurality. In some cases, the type IIs restriction enzyme is BsaI. In some cases, the gRNA in each gRNA-tRNA sequence part from the plurality is a single guide RNA (sgRNA). In some cases, the spacer sequence in each gRNA-tRNA sequence part from the plurality of gRNA-tRNA sequence parts comprises sequence complementary to a target sequence present at a different locus in a genetic element in a host cell than the spacer sequence in each other gRNA-tRNA sequence part from the plurality. In some cases, the plasmid backbone further comprises a scaffold sequence 3′ to the second type IIs restriction site, wherein the scaffold sequence comprises sequence necessary to bind to an RNA-guided DNA endonuclease. In some cases, one of the gRNA-tRNA sequence parts from the plurality differs from each other gRNA-tRNA sequence part from the plurality in that said gRNA-tRNA sequence part comprises, from 5′ to 3′, the first type IIs restriction site, a spacer sequence and the second type IIS restriction site. In some cases, each gRNA-tRNA sequence part from the plurality of gRNA-tRNA sequence parts is represented by a pool of gRNA-tRNA sequence parts. In some cases, each gRNA-tRNA sequence part within a pool comprises a spacer sequence that comprises sequence complementary to a target sequence present at an identical locus in a genetic element in a host cell as the spacer sequence in each other gRNA-tRNA sequence part within the pool. In some cases, each gRNA-tRNA sequence part within a pool comprises a spacer sequence that comprises sequence complementary to a target sequence present at a different locus in a genetic element in a host cell as the spacer sequence in each other gRNA-tRNA sequence part within the pool. In some cases, each gRNA-tRNA sequence part within a pool comprises a spacer sequence that comprises sequence complementary to a target sequence present at a different locus in a genetic element in a host cell than the spacer sequence in each gRNA-tRNA sequence part from each other pool.

In yet another aspect, provided herein is a method for preparing a plasmid comprising an array of guide RNA (gRNA)-tRNA sequences in tandem arrangement, the method comprising: (a) incubating under conditions that allow for digestion, a mixture comprising: (i) a type IIs restriction enzyme, (ii) a plasmid backbone comprising, from 5′ to 3′, a promoter sequence, a first type IIs restriction site, a stuffer sequence and a second Type IIs restriction site, wherein the first and the second type IIs restriction site are recognized and digested by the type IIs restriction enzyme of (i), and (iii) a plurality of gRNA-tRNA sequence parts, wherein each gRNA-tRNA sequence part in the plurality, comprises, from 5′ to 3′, a first type IIs restriction site, a gRNA and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality, and wherein the first and the second type IIs restriction site in each gRNA-tRNA sequence part in the plurality are recognized and digested by the type IIs restriction enzyme of (i), wherein digestion of the plasmid backbone of (i) removes the stuffer sequence and generates a first end within the promoter sequence and a second end distal to the first end, while digestion of each gRNA-tRNA sequence part within the plurality generates opposing first and second ends on each gRNA-tRNA sequence part that are different than the opposing first and second ends on each other gRNA-tRNA sequence part in the plurality, and wherein digestion of one gRNA-tRNA sequence part from the plurality generates a first end comprising sequence complementary to the first end of the plasmid backbone, while digestion for each other gRNA-tRNA sequence part from the plurality generates a first end that comprises sequence complementary to the second end of a gRNA-tRNA cleavage sequence part from each other gRNA-tRNA sequence part, and wherein digestion of one gRNA-tRNA sequence part from the plurality generates a second end comprising sequence complementary to the second end of the plasmid backbone; and (b) incubating the mixture with a ligase under conditions that allow for hybridization and covalent joining of first and/or second ends that comprise complementary sequence present within the mixture, wherein ligation operably links the promoter sequence in the plasmid backbone to an array of gRNA-tRNA sequence parts from the plurality of gRNA-tRNA sequence parts in tandem arrangement, thereby generating an assembled plasmid comprising an array of gRNA-tRNA sequences in tandem arrangement. In some cases, the promoter sequence is a Pol III promoter. In some cases, the promoter sequence is a pSNR52 promoter. In some cases, the second type IIs restriction site in each gRNA-tRNA sequence part from the plurality is within the 3′ terminus of the tRNA sequence. In some cases, the tRNA sequence in each gRNA-tRNA sequence part from the plurality comprises one or more of the group consisting of a pretRNA acceptor stem, a D-loop arm and a TψC-loop arm. In some cases, the tRNA sequence in each gRNA-tRNA sequence part from the plurality comprises an active site for one or more of the group consisting of RNase P, RNase Z, RNase E, RNase F and RNase D. In some cases, the tRNA sequence in each gRNA-tRNA sequence part from the plurality comprises a full pre-tRNA sequence. In some cases, the pre-tRNA sequence is selected from tRNA-ser, tRNA-gln, tRNA-lys or tRNA-gly. In some cases, the type IIs restriction enzyme is BsaI. In some cases, the gRNA in each gRNA-tRNA sequence part from the plurality is a single guide RNA (sgRNA). In some cases, the spacer sequence in each gRNA-tRNA sequence part from the plurality of gRNA-tRNA sequence parts comprises sequence complementary to a target sequence present at a different locus in a genetic element in a host cell than the spacer sequence in each other gRNA-tRNA sequence part from the plurality. In some cases, the plasmid backbone further comprises a scaffold comprising sequence necessary to bind to an RNA-guided DNA endonuclease 3′ to the second type IIs restriction site. In some cases, the one gRNA-tRNA sequence part from the plurality whose second end comprises sequence complementary to the second end of the plasmid backbone following digestion with the type IIS restriction enzyme of (i) differs from each other gRNA-tRNA sequence part from the plurality in that said gRNA-tRNA sequence part comprises, from 5′ to 3′, the first type IIS restriction site, a spacer sequence and the second type IIs restriction site. In some cases, each gRNA-tRNA sequence part from the plurality of gRNA-tRNA sequence parts is represented by a pool of gRNA-tRNA sequence parts, thereby generating a library of assembled plasmids comprising an array of gRNA-tRNA sequences in tandem arrangement. In some cases, each gRNA-tRNA sequence part within a pool comprises a spacer sequence that comprises sequence complementary to a target sequence present at an identical locus in a genetic element in a host cell as the spacer sequence in each other gRNA-tRNA sequence part within the pool. In some cases, each gRNA-tRNA sequence part within a pool comprises a spacer sequence that comprises sequence complementary to a target sequence present at a different locus in a genetic element in a host cell as the spacer sequence in each other gRNA-tRNA sequence part within the pool. In some cases, each gRNA-tRNA sequence part within a pool comprises a spacer sequence that comprises sequence complementary to a target sequence present at a different locus in a genetic element in a host cell than the spacer sequence in each gRNA-tRNA sequence part from each other pool. In some cases, the method further comprises (c) propagating the assembled plasmid comprising an array of gRNA-tRNA sequences in tandem arrangement in a microbial cell; and (d) isolating nucleic acid from the microbial cell of step (c), wherein the isolated nucleic acid comprises the assembled plasmid. In some cases, the method further comprises (c) propagating each of the assembled plasmids comprising an array of gRNA-tRNA sequences in tandem arrangement from the library in a microbial cell; and (d) isolating nucleic acid from each of the microbial cells of step (c), wherein the isolated nucleic acid from each of the microbial cells comprises an assembled plasmid from the library. In some cases, the propagating of step (c) entails transforming the microbial cell and growing in growth media. In some cases, the microbial cell is E. coli. In some cases, step (d) comprises picking transformants from step (c). In some cases, the method further comprises (e) sequencing nucleic acid isolated from the transformants from step (d).

In one aspect, provided herein is a method for editing the genome of a host cell, the method comprising: (a) introducing an assembled plasmid comprising an array of guide RNA (gRNA)-tRNA sequences in tandem arrangement generated using any method provided herein into a host cell, wherein the host cell expresses an RNA-guided DNA endonuclease or an RNA-guided DNA endonuclease is introduced into the host cell along with the assembled plasmid and wherein the host cell utilizes the tRNA sequence in each gRNA-tRNA sequence in the array to release each gRNA from the array of gRNA-tRNA sequences in tandem arrangement; and (b) introducing a plurality of repair fragments under conditions that allow for homology-directed repair (HDR) utilizing the RNA-guided DNA endonuclease, wherein the plurality of repair fragments comprises a repair fragment for each gRNA released in step (a) that comprises homology arms on opposing ends of the repair fragment that comprise sequence complementary to the locus targeted by the gRNA and at least one genetic edit, thereby editing the genome of the host cell. In some cases, the at least one genetic edit is selected from the group consisting of a substitution, an inversion, an insertion, a deletion, a single nucleotide polymorphism, and any combination thereof. In some cases, each repair fragment from the plurality of repair fragments is provided on a plasmid or as a linear fragment. In some cases, each repair fragment from the plurality of repair fragments is provided as a single-stranded DNA (ssDNA) or double-stranded DNA (dsDNA) linear fragment. In some cases, each repair fragment from the plurality of repair fragments is represented by a pool of repair fragments. In some cases, the at least one genetic edit within each repair fragment in the pool of repair fragment is different than the at least one genetic edit in each other repair fragment in the pool of repair fragments. In some cases, the assembled plasmid comprising an array of gRNA-tRNA sequences in tandem arrangement further comprises a selectable marker gene. In some cases, the assembled plasmid comprising an array of gRNA-tRNA sequences in tandem arrangement further comprises a centromere and autonomously replicating sequence. In some cases, the RNA-guided DNA endonuclease is selected from Cas9, Cas12a, Cas12b, Cas12c, Cas12d, Cas12e, Cas13a, Cas13b, Cas13c, Cpf1, and MAD7, or homologs, orthologs or paralogs thereof. In some cases, the RNA-guided DNA endonuclease is encoded on a plasmid, encoded in the genome of the host cell, translated from RNA, or introduced into the host cell as protein. In some cases, the host cell is a eukaryotic cell. In some cases, the host cell is a yeast cell. In some cases, the yeast cell is Saccharomyces cerevisiae. In some cases, the host cell is a filamentous fungus. In some cases, filamentous fungus is Aspergillus niger. In some cases, the host cell is a prokaryotic cell. In some cases, the prokaryotic host cell is Escherichia coli or Corynebacterium glutamicum. In some cases, the method for preparing an assembled plasmid comprising an array of guide RNA (gRNA)-tRNA sequences in tandem arrangement comprises (a) incubating under conditions that allow for digestion, a mixture comprising: (i) a type IIs restriction enzyme, (ii) a plasmid backbone comprising, from 5′ to 3′, a promoter sequence, a first type IIs restriction site, a stuffer sequence and a second Type IIs restriction site, wherein the first and the second type IIs restriction site are recognized and digested by the type IIs restriction enzyme of (i), and (iii) a plurality of gRNA-tRNA sequence parts, wherein each gRNA-tRNA sequence part in the plurality, comprises, from 5′ to 3′, a first type IIs restriction site, a gRNA and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality, and wherein the first and the second type IIs restriction site in each gRNA-tRNA sequence part in the plurality are recognized and digested by the type IIs restriction enzyme of (i), wherein digestion of the plasmid backbone of (i) removes the stuffer sequence and generates a first end within the promoter sequence and a second end distal to the first end, while digestion of each gRNA-tRNA sequence part within the plurality generates opposing first and second ends on each gRNA-tRNA sequence part that are different than the opposing first and second ends on each other gRNA-tRNA sequence part in the plurality, and wherein digestion of one gRNA-tRNA sequence part from the plurality generates a first end comprising sequence complementary to the first end of the plasmid backbone, while digestion for each other gRNA-tRNA sequence part from the plurality generates a first end that comprises sequence complementary to the second end of a gRNA-tRNA cleavage sequence part from each other gRNA-tRNA sequence part, and wherein digestion of one gRNA-tRNA sequence part from the plurality generates a second end comprising sequence complementary to the second end of the plasmid backbone; and (b) incubating the mixture with a ligase under conditions that allow for hybridization and covalent joining of first and/or second ends that comprise complementary sequence present within the mixture, wherein ligation operably links the promoter sequence in the plasmid backbone to an array of gRNA-tRNA sequence parts from the plurality of gRNA-tRNA sequence parts in tandem arrangement, thereby generating an assembled plasmid comprising an array of gRNA-tRNA sequences in tandem arrangement.

In another aspect, provided herein is a nucleic acid construct comprising two or more guide RNA (gRNA)-tRNA sequence units in tandem arrangement, wherein the tRNA sequence in each unit is different than the tRNA sequence in an adjacent unit, and wherein each gRNA in each gRNA-tRNA unit comprises a spacer sequence comprising sequence complementary to a locus in a genetic element in a host cell. In some cases, the tRNA sequence in each gRNA-tRNA sequence unit comprises one or more of the group consisting of a pretRNA acceptor stem, a D-loop arm and a TψC-loop arm. In some cases, the tRNA sequence in each gRNA-tRNA sequence unit comprises an active site for one or more of the group consisting of RNase P, RNase Z, RNase E, RNase F and RNase D. In some cases, the tRNA sequence in each gRNA-tRNA sequence part from the plurality comprises a full pre-tRNA sequence. In some cases, the pre-tRNA sequence is selected from tRNA-ser, tRNA-gln, tRNA-lys or tRNA-gly. In some cases, the gRNA in each gRNA-tRNA sequence unit is a single guide RNA (sgRNA). In some cases, the spacer sequence in each gRNA-tRNA sequence unit comprises sequence complementary to a target sequence present at a different locus in a genetic element in a host cell than the spacer sequence in each other gRNA-tRNA sequence unit. In some cases, the spacer sequence in each gRNA-tRNA sequence unit comprises sequence complementary to a target sequence present at an identical locus in a genetic element in a host cell as the spacer sequence in each other gRNA-tRNA sequence unit. In some cases, the nucleic acid construct further comprises a promoter sequence that is operably linked to the two or more gRNA-tRNA sequence units in tandem arrangement. In some cases, the nucleic acid construct further comprises a terminator sequence that is operably linked to the two or more gRNA-tRNA sequence units in tandem arrangement. In some cases, each of the two or more gRNA-tRNA sequence units comprises a promoter sequence and a terminator sequence operably linked thereto. In some cases, the promoter sequence is a Pol III promoter. In some cases, the terminator sequence is a Pol III terminator. In one embodiment, provided herein is an expression cassette comprising the nucleic acid construct. In one embodiment, provided herein is a vector comprising the expression cassette. In another embodiment, provided herein is a host cell comprising the nucleic acid construct. In yet another embodiment, provided herein is a genetically modified cell comprising a genetic edit, the cell having been edited by the introduction of the nucleic acid construct.

### BRIEF DESCRIPTION OF THE DRAWINGS

FIGS. 1A-1B illustrate parts for generating gRNA containing constructs comprising tRNA sequences interspersed therein for use in the genome editing methods provided herein. FIG. 1A shows a plasmid or vector backbone that contains type IIs restriction endonuclease sites (e.g., BsaI sites) for use in creating overhangs A and D (see partial nucleic acid sequences with SEQ ID NOs 1 and 2) in the promoter and gRNA backbone found within the plasmid, respectively. FIG. 1B shows nucleic acid parts (i.e., gRNA-tRNA sequence parts and spacer part; see partial nucleic acid sequences with SEQ ID NOs: 3-8) generated through DNA synthesis, PCR or a combination of the two that can be assembled into locations or slots within constructs in a modular fashion as provided herein. Parts for slot 1 contain a 5′ BsaI site that can create an overhang that will be complementary to overhang A generated within the SNR52 promoter in the plasmid backbone shown in FIG. 1A, while the 3′ BsaI site in slot 1 can create an overhang B in the 3′ end of tRNA-1. Parts for slot 2 have BsaI sites that can create 5′ overhang B and 3′ overhang C. The gRNA-tRNA parts that can be cloned into slot 3 or the last slot or location within a construct do not contain the gRNA backbone and have BsaI sites that can create 5′ overhang C and 3′ overhang D for ligation onto tRNA 2 and the plasmid backbone, respectively. A scarless cloning technique (e.g., Golden Gate (GG) cloning reaction) can be used to create a circular plasmid as shown in FIG. 1C. FIG. 1C shows the three (3) nucleic acid parts from FIG. 1B separated by different tRNAs assembled into the plasmid backbone shown in FIG. 1A. The nucleic acid parts can be assembled directly into a plasmid backbone that is a Cas9 containing plasmid, or into a separate cloning plasmid and then amplified by PCR and transformed directly into yeast as a linear piece of DNA with homology arms for integration into a yeast plasmid. Importantly, while this figure and the examples provided herein demonstrate multiplexed editing of three (3) loci, one may use the approach to edit many loci simultaneously—limited only by the number of tRNAs with unique 3′ overhangs available within the genome of a host cell. The same strategy can be used to create arrays of more than three (3) gRNAs by using additional parts similar to the slot 2 part with different tRNAs.

FIGS. 2A-2B illustrate results of functional testing and validation of tRNAs in a multiplex editing assay using four (4) different tRNAs assembled into duplex gRNA arrays as shown in FIG. 2A with spacers targeting trp1, lys2, leu2 and his3 loci in configurations described in FIG. 2B.

FIGS. 3A-3B illustrate results on use of multiplex genome editing using gRNA-tRNA assemblies comprising three (3) gRNAs targeting three (3) separate genomic loci in a host cell. FIG. 3A shows triplex gRNA-tRNA arrays with varied gRNA and tRNA parts as described by Table 1 for use in the multiplex genome editing method. NT: Non-targeting guide. FIG. 3B shows efficient triplex editing of Leu2, Lys2 and Trp1 loci observed using arrays of gRNAs and tRNAs. Lysine, serine and glycine tRNAs supported efficient gRNA expression and multiplexed genome editing with both plasmid-based (pBrew4) and integrated (pGuide2 & pGuide7) Cas9 expression.

FIGS. 4A-4B illustrate pooled assembly of gRNA-tRNA arrays in a single reaction or pot. FIG. 4A shows the plasmid backbone (see partial nucleic acid sequences with SEQ ID NOs 1 and 2) and gRNA-tRNA parts (see partial nucleic acid sequences with SEQ ID NOs 3-8) for use in the pooled assembly, while FIG. 4B shows the combinatorial assembly of nine (9) gRNAs into a 3× multiplexing gRNA-tRNA array.

FIG. 5 illustrates a scheme for multiplexed CRISPR based genome editing using multiple repair templates in combination with a multiplexed gRNA-tRNA array. A multiple gRNA expression cassette can be used to stably express multiple gRNAs from a Cas9 expressing plasmid. A host cell such as S. cerevisiae can be transformed with a multiple gRNA and Cas9 expression plasmid. The transformation can include multiple repair templates for each locus targeted by a gRNA. A diverse pool of strains with different combinations of alleles at each edited locus can be produced following this transformation procedure.

FIGS. 6A-6B illustrate results of a proof-of-concept assay for multiplexed CRISPR based genome editing using a pool of repair templates. FIG. 6A shows gRNA expression constructs and Lys2 and Trp1 repair fragments for use in the multiplexed CRISPR based genome editing method described in Example 2. Lys2 and Trp1 were simultaneously edited with 4 and 3 repair templates integrating payloads of varied length, respectively. gRNAs targeting both genes were expressed from a single promoter and processed into separate gRNAs using the tRNA processing RNases P and Z. Repair templates were provided in the transformation introducing insertions of varied length. FIG. 6B shows genotyping of multiplexed pooled repair fragment colonies. All possible combinations of edited alleles were recovered by the experiments described in Example 2. Colonies were genotyped by PCR to determine the insertion size in each colony.

FIG. 7 illustrates an embodiment of a gRNA/tRNA comprising unit or part within a nucleic construct comprising a plurality of gRNAs interspersed with tRNA sequences at regular intervals as provided herein where the gRNA/tRNA part is present as an individual expression cassette that comprises a promoter sequence and terminator sequence operably linked thereto.

### DETAILED DESCRIPTION

### Definitions

While the following terms are believed to be well understood by one of ordinary skill in the art, the following definitions are set forth to facilitate explanation of the presently disclosed subject matter.

As used herein, the term “a” or “an” can refer to one or more of that entity, i.e. can refer to plural referents. As such, the terms “a” or “an”, “one or more” and “at least one” can be used interchangeably herein. In addition, reference to “an element” by the indefinite article “a” or “an” does not exclude the possibility that more than one of the elements is present, unless the context clearly requires that there is one and only one of the elements.

Unless the context requires otherwise, throughout the present specification and claims, the word “comprise” and variations thereof, such as, “comprises” and “comprising” are to be construed in an open, inclusive sense that is as “including, but not limited to”.

Reference throughout this specification to “one embodiment” or “an embodiment” means that a particular feature, structure or characteristic described in connection with the embodiment may be included in at least one embodiment of the present disclosure. Thus, the appearances of the phrases “in one embodiment” or “in an embodiment” in various places throughout this specification may not necessarily all referring to the same embodiment. It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination.

As used herein, the terms “cellular organism” “microorganism” or “microbe” should be taken broadly. These terms are used interchangeably and include, but are not limited to, the two prokaryotic domains, Bacteria and Archaea, as well as certain eukaryotic fungi and protists. In some embodiments, the disclosure refers to the “microorganisms” or “cellular organisms” or “microbes” of lists/tables and figures present in the disclosure. This characterization can refer to not only the identified taxonomic genera of the tables and figures, but also the identified taxonomic species, as well as the various novel and newly identified or designed strains of any organism in said tables or figures. The same characterization holds true for the recitation of these terms in other parts of the Specification, such as in the Examples.

As used herein, the term “prokaryotes” is art recognized and refers to cells that contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

As used herein, the term “Archaea” refers to a categorization of organisms of the division Mendosicutes, typically found in unusual environments and distinguished from the rest of the prokaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt (NaCl); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

As used herein, “bacteria” or “eubacteria” can refer to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

As used herein, a “eukaryote” is any organism whose cells contain a nucleus and other organelles enclosed within membranes. Eukaryotes belong to the taxon Eukarya or Eukaryota. The defining feature that sets eukaryotic cells apart from prokaryotic cells (the aforementioned Bacteria and Archaea) is that they have membrane-bound organelles, especially the nucleus, which contains the genetic material, and is enclosed by the nuclear envelope.

As used herein, the terms “genetically modified host cell,” “recombinant host cell,” and “recombinant strain” are used interchangeably herein and can refer to host cells that have been genetically modified by the cloning and transformation methods of the present disclosure. Thus, the terms include a host cell (e.g., bacteria, yeast cell, fungal cell, CHO, human cell, etc.) that has been genetically altered, modified, or engineered, such that it exhibits an altered, modified, or different genotype and/or phenotype (e.g., when the genetic modification affects coding nucleic acid sequences of the microorganism), as compared to the naturally-occurring organism from which it was derived. It is understood that in some embodiments, the terms refer not only to the particular recombinant host cell in question, but also to the progeny or potential progeny of such a host cell

As used herein, the term “wild-type microorganism” or “wild-type host cell” can describe a cell that occurs in nature, i.e. a cell that has not been genetically modified.

As used herein, the term “genetically engineered” may refer to any manipulation of a host cell's genome (e.g. by insertion, deletion, mutation, or replacement of nucleic acids).

As used herein, the term “control” or “control host cell” can refer to an appropriate comparator host cell for determining the effect of a genetic modification or experimental treatment. In some embodiments, the control host cell is a wild type cell. In other embodiments, a control host cell is genetically identical to the genetically modified host cell, save for the genetic modification(s) differentiating the treatment host cell. In some embodiments, the present disclosure teaches the use of parent strains as control host cells (e.g., the S₁ strain that was used as the basis for the strain improvement program). In other embodiments, a host cell may be a genetically identical cell that lacks a specific promoter or SNP being tested in the treatment host cell.

As used herein, the term “allele(s)” can mean any of one or more alternative forms of a gene, all of which alleles relate to at least one trait or characteristic. In a diploid cell, the two alleles of a given gene occupy corresponding loci on a pair of homologous chromosomes.

As used herein, the term “locus” (loci plural) can mean any site at which an edit to the native genomic sequence is desired. In one embodiment, said term can mean a specific place or places or a site on a chromosome where for example a gene or genetic marker is found.

As used herein, the term “genetically linked” can refer to two or more traits that are co-inherited at a high rate during breeding such that they are difficult to separate through crossing.

A “recombination” or “recombination event” as used herein can refer to a chromosomal crossing over or independent assortment.

As used herein, the term “phenotype” can refer to the observable characteristics of an individual cell, cell culture, organism, or group of organisms, which results from the interaction between that individual's genetic makeup (i.e., genotype) and the environment.

As used herein, the term “chimeric” or “recombinant” when describing a nucleic acid sequence or a protein sequence can refer to a nucleic acid, or a protein sequence, that links at least two heterologous polynucleotides, or two heterologous polypeptides, into a single macromolecule, or that rearranges one or more elements of at least one natural nucleic acid or protein sequence. For example, the term “recombinant” can refer to an artificial combination of two otherwise separated segments of sequence, e.g., by chemical synthesis or by the manipulation of isolated segments of nucleic acids by genetic engineering techniques.

As used herein, a “synthetic nucleotide sequence” or “synthetic polynucleotide sequence” is a nucleotide sequence that is not known to occur in nature or that is not naturally occurring. Generally, such a synthetic nucleotide sequence can comprise at least one nucleotide difference when compared to any other naturally occurring nucleotide sequence.

As used herein, the term “nucleic acid” can refer to a polymeric form of nucleotides of any length, either ribonucleotides or deoxyribonucleotides, or analogs thereof. This term can refer to the primary structure of the molecule, and thus includes double- and single-stranded DNA, as well as double- and single-stranded RNA. It also includes modified nucleic acids such as methylated and/or capped nucleic acids, nucleic acids containing modified bases, backbone modifications, and the like. The terms “nucleic acid” and “nucleotide sequence” are used interchangeably.

As used herein, the term “gene” can refer to any segment of DNA associated with a biological function. Thus, genes can include, but are not limited to, coding sequences and/or the regulatory sequences required for their expression. Genes can also include non-expressed DNA segments that, for example, form recognition sequences for other proteins. Genes can be obtained from a variety of sources, including cloning from a source of interest or synthesizing from known or predicted sequence information, and may include sequences designed to have desired parameters.

As used herein, the term “homologous” or “homologue” or “ortholog” or “orthologue” is known in the art and can refer to related sequences that share a common ancestor or family member and are determined based on the degree of sequence identity.

The terms “homology,” “homologous,” “substantially similar” and “corresponding substantially” can be used interchangeably herein. Said terms can refer to nucleic acid fragments wherein changes in one or more nucleotide bases do not affect the ability of the nucleic acid fragment to mediate gene expression or produce a certain phenotype. These terms can also refer to modifications of the nucleic acid fragments of the instant disclosure such as deletion or insertion of one or more nucleotides that do not substantially alter the functional properties of the resulting nucleic acid fragment relative to the initial, unmodified fragment. It is therefore understood, as those skilled in the art will appreciate, that the disclosure encompasses more than the specific exemplary sequences. These terms describe the relationship between a gene found in one species, subspecies, variety, cultivar or strain and the corresponding or equivalent gene in another species, subspecies, variety, cultivar or strain. For purposes of this disclosure homologous sequences are compared.

“Homologous sequences” or “homologues” or “orthologs” are thought, believed, or known to be functionally related. A functional relationship may be indicated in any one of a number of ways, including, but not limited to: (a) degree of sequence identity and/or (b) the same or similar biological function. Preferably, both (a) and (b) are indicated. Sequence homology between amino acid or nucleic acid sequences can be defined in terms of shared ancestry. Two segments of nucleic acid can have shared ancestry because of either a speciation event (orthologs) or a duplication event (paralogs). Homology among amino acid or nucleic acid sequences can be inferred from their sequence similarity such that amino acid or nucleic acid sequences are said to be homologous is said amino acid or nucleic acid sequences share significant similarity. Significant similarity can be strong evidence that two sequences are related by divergent evolution from a common ancestor. Alignments of multiple sequences can be used to discover the homologous regions. Homology can be determined using software programs readily available in the art, such as those discussed in Current Protocols in Molecular Biology (F. M. Ausubel et al., eds., 1987) Supplement 30, section 7.718, Table 7.71. Some alignment programs are BLAST (NCBI), MacVector (Oxford Molecular Ltd, Oxford, U.K.), ALIGN Plus (Scientific and Educational Software, Pennsylvania) and AlignX (Vector NTI, Invitrogen, Carlsbad, Calif.). Another alignment program is Sequencher (Gene Codes, Ann Arbor, Mich.), using default parameters.

As used herein, the term “endogenous” or “endogenous gene,” can refer to the naturally occurring gene, in the location in which it is naturally found within the host cell genome. In the context of the present disclosure, operably linking a heterologous promoter to an endogenous gene means genetically inserting a heterologous promoter sequence in front of an existing gene, in the location where that gene is naturally present. An endogenous gene as described herein can include alleles of naturally occurring genes that have been mutated according to any of the methods of the present disclosure.

As used herein, the term “exogenous” can be used interchangeably with the term “heterologous,” and refers to a substance coming from some source other than its native source. For example, the terms “exogenous protein,” or “exogenous gene” refer to a protein or gene from a non-native source or location, and that have been artificially supplied to a biological system.

As used herein, the term “nucleotide change” refers to, e.g., nucleotide substitution, deletion, and/or insertion, as is well understood in the art. For example, mutations can contain alterations that produce silent substitutions, additions, or deletions, but do not alter the properties or activities of the encoded protein or how the proteins are made. Alternatively, mutations can be nonsynonymous substitutions or changes that can alter the amino acid sequence of the encoded protein and can result in an alteration in properties or activities of the protein.

As used herein, the term “protein modification” can refer to, e.g., amino acid substitution, amino acid modification, deletion, and/or insertion, as is well understood in the art.

As used herein, the term “at least a portion” or “fragment” of a nucleic acid or polypeptide can mean a portion having the minimal size characteristics of such sequences, or any larger fragment of the full-length molecule, up to and including the full-length molecule. A fragment of a polynucleotide of the disclosure may encode a biologically active portion of a genetic regulatory element. A biologically active portion of a genetic regulatory element can be prepared by isolating a portion of one of the polynucleotides of the disclosure that comprises the genetic regulatory element and assessing activity as described herein. Similarly, a portion of a polypeptide may be 4 amino acids, 5 amino acids, 6 amino acids, 7 amino acids, and so on, going up to the full-length polypeptide. The length of the portion to be used can depend on the particular application. A portion of a nucleic acid useful as a hybridization probe may be as short as 12 nucleotides; in some embodiments, it is 20 nucleotides. A portion of a polypeptide useful as an epitope may be as short as 4 amino acids. A portion of a polypeptide that performs the function of the full-length polypeptide would generally be longer than 4 amino acids.

Variant polynucleotides can also encompass sequences derived from a mutagenic and recombinogenic procedure such as DNA shuffling. Strategies for such DNA shuffling are known in the art. See, for example, Stemmer (1994) PNAS 91:10747-10751; Stemmer (1994) Nature 370:389-391; Crameri et al., (1997) Nature Biotech. 15:436-438; Moore et al., (1997) J. Mol. Biol. 272:336-347; Zhang et al., (1997) PNAS 94:4504-4509; Crameri et al., (1998) Nature 391:288-291; and U.S. Pat. Nos. 5,605,793 and 5,837,458.

For PCR amplifications disclosed herein, oligonucleotide primers can be designed for use in PCR reactions to amplify corresponding DNA sequences from cDNA or genomic DNA extracted from any organism of interest. Methods for designing PCR primers and PCR cloning are generally known in the art and are disclosed in Sambrook et al., (2001) Molecular Cloning: A Laboratory Manual (3^{rd} ed., Cold Spring Harbor Laboratory Press, Plainview, N.Y.). See also Innis et al., eds. (1990) PCR Protocols: A Guide to Methods and Applications (Academic Press, New York); Innis and Gelfand, eds. (1995) PCR Strategies (Academic Press, New York); and Innis and Gelfand, eds. (1999) PCR Methods Manual (Academic Press, New York). Known methods of PCR include, but are not limited to, methods using paired primers, nested primers, single specific primers, degenerate primers, gene-specific primers, vector-specific primers, partially-mismatched primers, and the like.

The term “primer” as used herein can refer to an oligonucleotide which is capable of annealing to the amplification target allowing a DNA polymerase to attach, thereby serving as a point of initiation of DNA synthesis when placed under conditions in which synthesis of primer extension product is induced, i.e., in the presence of nucleotides and an agent for polymerization such as DNA polymerase and at a suitable temperature and pH. The (amplification) primer can be single stranded for maximum efficiency in amplification. The primer can be an oligodeoxyribonucleotide. The primer must be sufficiently long to prime the synthesis of extension products in the presence of the agent for polymerization. The exact lengths of the primers will depend on many factors, including temperature and composition (A/T vs. G/C content) of primer. A pair of bi-directional primers consists of one forward and one reverse primer as commonly used in the art of DNA amplification such as in PCR amplification.

As used herein, “promoter” can refer to a DNA sequence capable of controlling the expression of a coding sequence or functional RNA. In some embodiments, the promoter sequence consists of proximal and more distal upstream elements, the latter elements often referred to as enhancers. Accordingly, an “enhancer” can be a DNA sequence that can stimulate promoter activity and may be an innate element of the promoter or a heterologous element inserted to enhance the level or tissue specificity of a promoter. Promoters may be derived in their entirety from a native gene or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Any type of promoter can be operably linked to a nucleic acid construct or expression cassette provided herein. Examples of promoters can include, without limitation, tissue-specific promoters, constitutive promoters, inducible promoters, and promoters responsive or unresponsive to a particular stimulus. In other embodiments, a promoter that facilitates the expression of a nucleic acid molecule without significant tissue or temporal-specificity can be used (i.e., a constitutive promoter). For example, a beta-actin promoter such as the chicken beta-actin gene promoter, ubiquitin promoter, miniCAGs promoter, glyceraldehyde-3-phosphate dehydrogenase (GAPDH) promoter, or 3-phosphoglycerate kinase (PGK) promoter can be used, as well as viral promoters such as the herpes simplex virus thymidine kinase (HSV-TK) promoter, the SV40 promoter, or a cytomegalovirus (CMV) promoter. In some embodiments, a fusion of the chicken beta actin gene promoter and the CMV enhancer is used as a promoter. See, for example, Xu et al. (2001) Hum. Gene Ther. 12:563; and Kiwaki et al. (1996) Hum. Gene Ther. 7:821.

As used herein, the phrases “recombinant construct”, “expression construct”, “chimeric construct”, “construct”, and “recombinant DNA construct” are used interchangeably herein. A recombinant construct can comprise an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source but arranged in a manner different than that found in nature. Such construct may be used by itself or may be used in conjunction with a vector. If a vector is used, then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the disclosure. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by direct sequencing, Southern analysis of DNA, Northern analysis of mRNA expression, immunoblotting analysis of protein expression, or phenotypic analysis, among others. Vectors can be plasmids, viruses, bacteriophages, pro-viruses, phagemids, transposons, artificial chromosomes, and the like, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that is not autonomously replicating. As used herein, the term “expression” refers to the production of a functional end-product e.g., an mRNA or a protein (precursor or mature).

“Operably linked” or “functionally linked” can mean the sequential arrangement of any functional payload according to the disclosure (e.g., promoter, terminator, degron, solubility tag, etc.) with a further oligo- or polynucleotide. In some cases, the sequential arrangement can result in transcription of said further polynucleotide. In some cases, the sequential arrangement can result in translation of said further polynucleotide. The functional payloads can be present upstream or downstream of the further oligo or polynucleotide. In one example, “operably linked” or “functionally linked” can mean a promoter controls the transcription of the gene adjacent or downstream or 3′ to said promoter. In another example, “operably linked” or “functionally linked” can mean a terminator controls termination of transcription of the gene adjacent or upstream or 5′ to said terminator.

The term “product of interest” or “biomolecule” as used herein can refer to any product produced by microbes from feedstock. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a microbe, such as: a microbial enzyme, including: catalase, amylase, protease, pectinase, glucose isomerase, cellulase, hemicellulase, lipase, lactase, streptokinase, and many others. The intracellular component may also include recombinant proteins, such as insulin, hepatitis B vaccine, interferon, granulocyte colony-stimulating factor, streptokinase and others.

As used herein, the term “HTP genetic design library” or “library” can refer to collections of genetic perturbations according to the present disclosure. In some embodiments, the libraries of the present disclosure may manifest as (i) a collection of sequence information in a database or other computer file, (ii) a collection of genetic constructs encoding for the aforementioned series of genetic elements, or (iii) host cell strains comprising said genetic elements. In some embodiments, the libraries of the present disclosure may refer to collections of individual elements (e.g., tRNA-gRNA expression cassettes, tRNA-gRNA arrays, gRNA-tRNA expression cassettes, gRNA-tRNA arrays, collections of promoters for PRO swap libraries, collections of terminators for STOP swap libraries, collections of protein solubility tags for SOLUBILITY TAG swap libraries, or collections of protein degradation tags for DEGRADATION TAG swap libraries). In other embodiments, the libraries of the present disclosure may also refer to combinations of genetic elements, such as combinations of promoter:genes, gene:terminator, or even promoter:gene:terminators. In some embodiments, the libraries of the present disclosure may also refer to combinations of promoters, terminators, protein solubility tags and/or protein degradation tags. In some embodiments, the libraries of the present disclosure further comprise meta-data associated with the effects of applying each member of the library in host organisms. For example, a library as used herein can include a collection of promoter:gene sequence combinations, together with the resulting effect of those combinations on one or more phenotypes in a particular species, thus improving the future predictive value of using said combination in future promoter swaps.

As used herein, the term “SNP” refers to Small Nuclear Polymorphism(s). In some embodiments, SNPs of the present disclosure should be construed broadly, and include single nucleotide polymorphisms, sequence insertions, deletions, inversions, and other sequence replacements. As used herein, the term “non-synonymous” or non-synonymous SNPs” refers to mutations that lead to coding changes in host cell proteins

A “high-throughput (HTP)” method of genomic engineering may involve the utilization of at least one piece of automated equipment (e.g. a liquid handler or plate handler machine) to carry out at least one-step of said method.

The term “polynucleotide” as used herein encompasses oligonucleotides and refers to a nucleic acid of any length. Polynucleotides may be DNA or RNA. Polynucleotides may be single-stranded (ss) or double-stranded (ds) unless otherwise specified. Polynucleotides may be synthetic, for example, synthesized in a DNA synthesizer, or naturally occurring, for example, extracted from a natural source, or derived from cloned or amplified material. Polynucleotides referred to herein can contain modified bases or nucleotides.

The term “pool”, as used herein, can refer to a collection of at least 2 polynucleotides. In some embodiments, a set of polynucleotides may comprise at least 5, at least 10, at least 12 or at least 15 or more polynucleotides. In some embodiments, a set of polynucleotides may comprise at least 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 2000, 3000, 4000, 5000, 6000, 7000, 8000, 9000, 10000 or more polynucleotides.

As used herein, the term “assembling”, can refer to a reaction in which two or more, four or more, six or more, eight or more, ten or more, 12 or more 15 or more polynucleotides, e.g., four or more polynucleotides are joined to another to make a longer polynucleotide.

As used herein, the term “incubating under suitable reaction conditions”, can refer to maintaining a reaction at a suitable temperature and time to achieve the desired results, i.e., polynucleotide assembly. Reaction conditions suitable for the enzymes and reagents used in the present method are known (e.g. as described in the Examples herein) and, as such, suitable reaction conditions for the present method can be readily determined. These reactions conditions may change depending on the enzymes used (e.g., depending on their optimum temperatures, etc.).

As used herein, the term “joining”, can refer to the production of covalent linkage between two sequences.

As used herein, the term “composition” can refer to a combination of reagents that may contain other reagents, e.g., glycerol, salt, dNTPs, etc., in addition to those listed. A composition may be in any form, e.g., aqueous or lyophilized, and may be at any state (e.g., frozen or in liquid form).

As used herein, a “vector” is a suitable DNA into which a fragment or nucleic acid assembly may be integrated such that the engineered vector can be replicated in a host cell. A vector as used herein can be referred to as an expression vector, or a vector system. A vector system as used herein can be set of components needed to bring about DNA insertion into a genome or other targeted DNA sequence such as an episome, plasmid, or even virus/phage DNA segment. Vector systems can be viral vectors (e.g., retroviruses, adeno-associated virus and integrating phage viruses), and non-viral vectors (e.g., transposons) used for gene delivery in animals. Vector as used herein can contain one or more expression cassettes that comprise one or more expression control sequences, wherein an expression control sequence can be a nucleic acid sequence that controls and regulates the transcription and/or translation of another DNA sequence or mRNA, respectively. The vector can be any vector, expression vector, or a vector system known in the art. The vector can be selected from a plasmid, episome, cosmid, and viral vector. A vector used in any of the methods, compositions or kits provided herein can be linearized. A linearized vector may be created by restriction endonuclease digestion of a circular vector or by PCR. The concentration of fragments and/or linearized vectors can be determined by gel electrophoresis or other means.

As used herein, the term “tRNA sequence” can refer to any portion of a pre-tRNA or tRNA precursor that is required to facilitate recognition and cleavage of both ends of said pre-tRNA or tRNA precursor by one or more enzymes of a tRNA processing system. In some cases, the tRNA sequence consist of a full pre-tRNA sequence. In some cases, the tRNA sequence consists of one or more portions of the pre-tRNA required to facilitate recognition and cleavage of said pre-tRNA or tRNA precursor portion by one or more enzymes of a tRNA processing system. The one or more portions can be selected from the group consisting of the acceptor stem, D-loop arm, TψC-loop arm and any combination thereof. The tRNA sequence can be a naturally occurring pre-tRNA sequence or portion thereof and can be endogenous or exogenous to a host cell used in any of the methods provided herein. The tRNA sequence can be a non-naturally occurring or mutated form of a pre-tRNA sequence or portion thereof such that said non-naturally occurring or mutated form of a pre-tRNA sequence or portion thereof retains the ability to be recognized and cleaved by one or more enzymes of a tRNA processing system. In some cases, the one or more enzymes of the tRNA processing system can be selected from RNase P, RNase Z, RNase E and any combination thereof. In some cases, the one or more enzymes of the tRNA processing system can be selected from RNase P, RNase F, RNase D and any combination thereof. The one or more enzymes of the tRNA processing system can be endogenous or exogenous to the host cell used in any of the methods provided herein.

The term “CRISPR RNA” or “crRNA” as used herein can refer to the RNA strand responsible for hybridizing with target DNA sequences, and/or recruiting CRISPR endonucleases. crRNAs may be naturally occurring or may be synthesized according to any known method of producing RNA.

The term “guide sequence” or “spacer” or “spacer sequence” as used herein can refer to the portion of a crRNA, guide RNA (gRNA) or single guide RNA (sgRNA) that is responsible for hybridizing with the target DNA.

The term “protospacer” as used herein can refer to the sequence targeted by a crRNA or guide sequence. In some embodiments, the protospacer sequence hybridizes with the crRNA/gRNA/sgRNA guide sequence of a CRISPR complex. In some cases, the protospacer can be referred to as a target sequence.

The term “seed region” as used herein can refer to the ribonucleic sequence responsible for initial complexation between a DNA sequence CRISPR ribonucleoprotein complex. Mismatches between the seed region and a target DNA sequence have a stronger effect on target site recognition and cleavage than the remainder of the crRNA/sgRNA sequence. In some embodiments a single mismatch in the seed region of a crRNA/gRNA can render a CRISPR complex inactive at that binding site. In some embodiments, the seed regions for Cas9 endonucleases are located along the last ˜12 nt of the 3′ portion of the guide sequence, which correspond (hybridize) to the portion of the protospacer target sequence that is adjacent to the PAM. In some embodiments, the seed regions for Cpf1 endonucleases are located along the first ˜5 nt of the 5′ portion of the guide sequence, which correspond (hybridize) to the portion of the protospacer target sequence adjacent to the PAM.

The term “tracrRNA” as used herein can refer to a small trans-encoded RNA. TracrRNA is complementary to and base pairs with crRNA to form a crRNA/tracrRNA hybrid, capable of recruiting CRISPR endonucleases to target sequences.

The term “Guide RNA” or “gRNA” as used herein refers to an RNA sequence or combination of sequences capable of recruiting a CRISPR endonuclease to a target sequence. Thus, as used herein, a guide RNA can be a natural or synthetic crRNA (e.g., for Cpf1), a natural or synthetic crRNA/tracrRNA hybrid (e.g., for Cas9), or a single-guide RNA (sgRNA).

### Overview

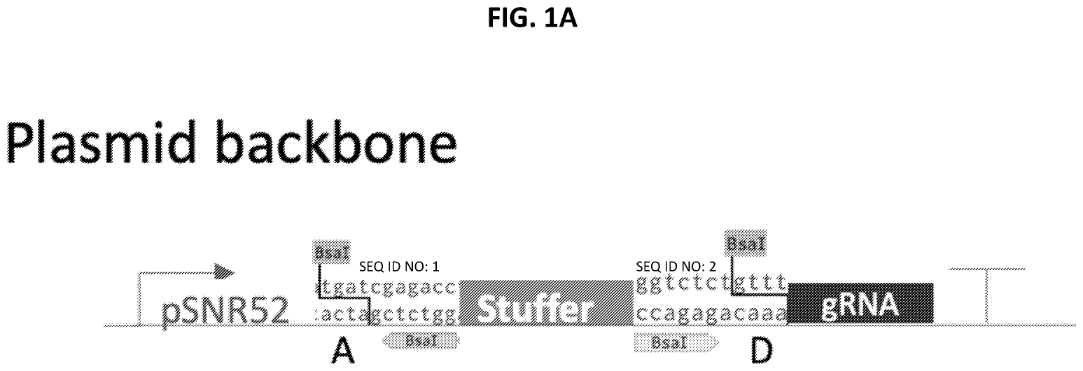

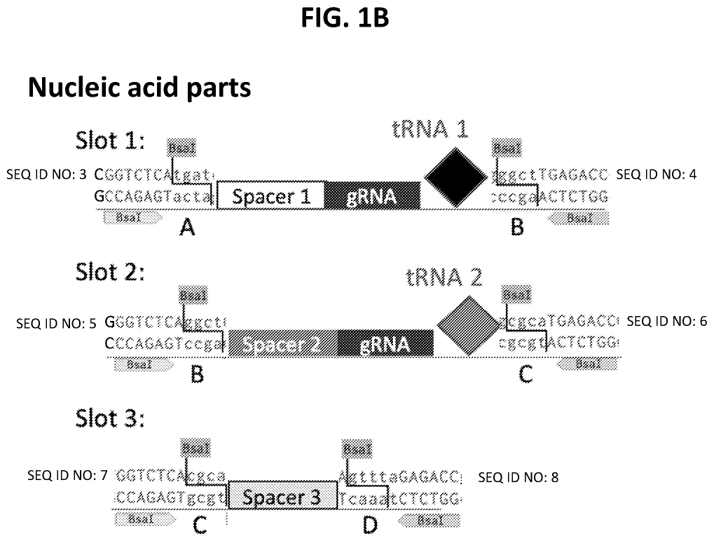

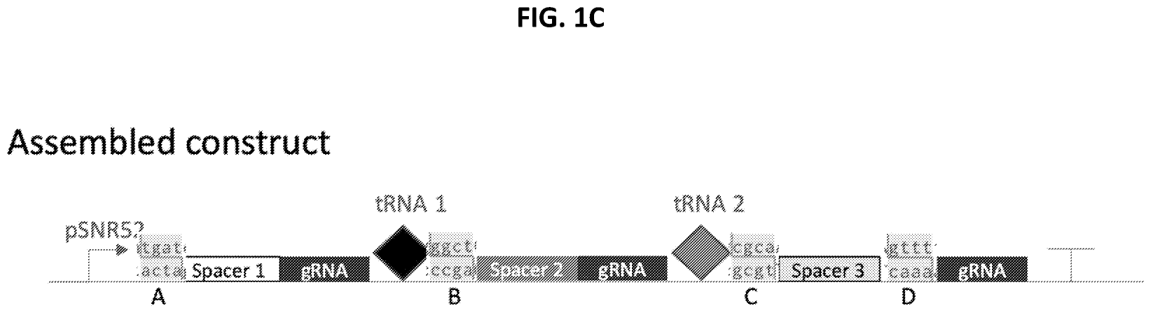

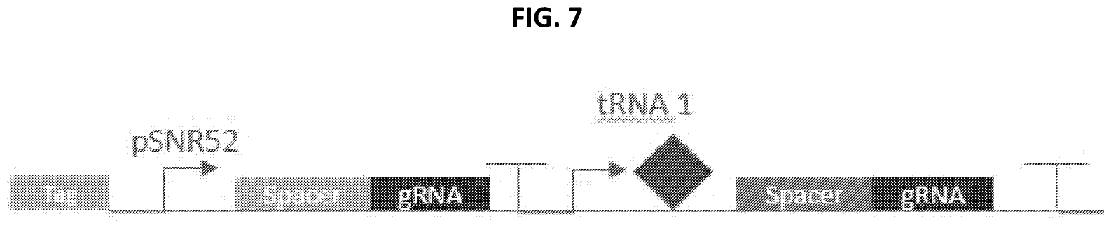

Provided herein is a nucleic acid construct comprising a plurality of guide RNAs (gRNAs) interspersed with tRNA sequences at regular intervals as well as kits and compositions comprising said nucleic acid construct and methods of producing said nucleic acid construct. The plurality of gRNAs interspersed with tRNA sequences in the construct can be arranged as units or parts. The units or parts can be arranged in a series or tandemly. In one embodiment, the plurality of gRNAs interspersed with tRNA sequences in the construct are arranged as a plurality of tRNA sequence-gRNA units or parts in the construct. The plurality of tRNA sequence-gRNA units can be at least, at most or exactly 2, 3, 4, 5, 6, 7, 8, 9 or 10 units arranged in series. In another embodiment, the plurality of gRNAs interspersed with tRNA sequences in the construct are arranged as a plurality of gRNA-tRNA sequence units or parts in the construct. The plurality of gRNA-tRNA sequence units can be at most or exactly 2, 3, 4, 5, 6, 7, 8, 9 or 10 units arranged in series. In one embodiment, a nucleic construct comprising a plurality of gRNAs interspersed with tRNA sequences at regular intervals as provided herein is present as an expression cassette that comprises a promoter sequence and terminator sequence operably linked thereto. An example of this embodiment can be found in FIG. 1C. In another embodiment, each gRNA/tRNA comprising unit or part within the nucleic construct comprising a plurality of gRNAs interspersed with tRNA sequences at regular intervals as provided herein is present as an individual expression cassette that comprises a promoter sequence and terminator sequence operably linked thereto. An example of this embodiment can be found in FIG. 7. In yet another embodiment, the nucleic acid construct comprises two or more expression cassettes such that each of the expression cassettes comprises a promoter and terminator operably linked to a plurality of gRNA-tRNA sequence units or tRNA sequence-gRNA units arranged in tandem. The plurality of tandemly arranged gRNA-tRNA sequence units operably linked to the promoter and terminator sequences within each expression cassette can be at least, at most or exactly 2, 3, 4 or 5 units. The plurality of tandemly arranged tRNA sequence-gRNA units operably linked to the promoter and terminator sequences within each expression cassette can be at least, at most or exactly 2, 3, 4 or 5 units. The nucleic acid construct can be present in or assembled into a vector. In one embodiment, the vector comprises a promoter and terminator such that assembly of the nucleic construct into the vector operably links the promoter and terminator of the vector to the nucleic acid construct. The vector can be any vector, expression vector, or a vector system known in the art. The vector can be selected from a plasmid, episome, cosmid, and viral vector. The promoter sequence can be a RNA polymerase II (Pol II) or RNA polymerase III (Pol III) promoter. In one embodiment, the promoter is a Pol III promoter. In one embodiment, the promoter is an SNR52 promoter. The terminator sequence can be a Pol III terminator.

In some cases, the tRNA sequence in each unit in a nucleic acid construct provided herein is different than the tRNA sequence in an adjacent unit. In some cases, the tRNA sequence in each unit is different than the tRNA sequence in each adjacent unit. In some cases, the tRNA sequence in each unit is different than the tRNA sequence that precedes and proceeds a respective unit. The tRNA sequence in each unit can be any tRNA sequence or portion thereof that can be recognized and processed (i.e., cleaved) by components of a tRNA processing system. The tRNA processing system can be endogenous to a host cell that contains the nucleic acid constructs or can be exogenously introduced into said cell. In one embodiment, the tRNA sequence in each unit includes one or more of the group consisting of a pretRNA acceptor stem, a D-loop arm and a TψC-loop arm. In one embodiment, the tRNA sequence in each unit includes an active site for one or more of the group consisting of RNase P, RNase Z and RNase E. In another embodiment, the tRNA sequence in each unit includes an active site for one or more of the group consisting of RNase P, RNase F and RNase D. In another embodiment, the tRNA sequence in each unit includes a full pre-tRNA sequence. The pre-tRNA sequence can be selected from tRNA-ser, tRNA-gln, tRNA-lys and tRNA-gly. In one embodiment, the pre-tRNA sequence is selected from tRNA-ser, tRNA-gln, tRNA-lys and tRNA-gly from S. cerevisiae. The tRNA sequences used in the nucleic constructs provided herein can be exogenous or endogenous to the host cell into which the nucleic acid construct is introduced.

Each gRNA in each gRNA/tRNA comprising unit in a nucleic acid construct comprising a plurality of guide RNAs (gRNAs) interspersed with tRNA sequences at regular intervals as provided herein can comprise a spacer sequence that comprises sequence complementary to a target or protospacer sequence present at a locus in a genetic element in a cell into which the nucleic acid construct can be introduced. In one embodiment, the spacer sequence in each unit comprises sequence complementary to a target sequence that is present at a different locus in a genetic element in a host cell than the target sequence targeted by the spacer sequence in each other unit. Further to this embodiment, the gRNAs in the nucleic acid construct can target multiple different genes in the host cell. In another embodiment, the spacer sequence in each unit comprises sequence complementary to a target sequence at an identical locus in a genetic element in a host cell as the target sequence complementary to the spacer sequence in each other unit. Further to this embodiment, the gRNAs in the nucleic acid construct can target multiple sites within a single gene in the host cell. In yet another embodiment, the spacer sequences in a subset of units within the nucleic acid construct comprise sequences complementary to target sequences at an identical locus in a genetic element in a host cell and yet target a different locus in the genetic element than the target sequences that comprise sequence complementary to the spacer sequences in each other unit not in said subset. The genetic element can be a chromosome, plasmid or cosmid. In one embodiment, the gRNA in each gRNA-tRNA sequence unit is a single guide RNA (sgRNA).

Also provided herein are methods for editing the genome of a host cell following introduction of a nucleic acid construct comprising a plurality of guide RNAs (gRNAs) interspersed with tRNA sequences at regular intervals as provided herein. Said methods can entail the use of an expression vector comprising a nucleic acid construct comprising a plurality of guide RNAs (gRNAs) interspersed with tRNA sequences at regular intervals as provided herein or compositions comprising the same in combination with one or a pool of repair fragments introduced into the host cell in order to facilitate homology-directed repair (HDR) using CRISPR. The use of different tRNA sequences within the nucleic acid construct as described herein can serve to increase the stability of the expression vector when present in a host cell as compared to similar expression vectors that contain identical tRNA sequences in an array of gRNAs interspersed with tRNA sequences. The methods provided herein can be used for single or multiplexed genome editing of a host or recipient cell. The introduction of multiple repair templates for each target locus can be used to increase the amount of genetic diversity produced in a single round of editing. For example, in a process aimed at varying the level of expression of three (3) genes by changing their promoter sequences, a ladder of six (6) promoters driving the expression of each targeted gene at varying levels of expression can be introduced as repair fragments in a single step. Using a combinatorial approach, this single transformation event can produce all 342 possible new genotypes. The genomic editing methods provided herein can be used with any nucleic acid guided nuclease and in any organism in which editing can be accomplished by homologous recombination with a nucleic acid (e.g., DNA) repair template. Any type of genetic edit that can be facilitated using homologous recombination can be made using the methods provided herein such as, for example, SNP swaps, promoter swaps, ORF insertions, pathway insertions, pathway deletions or combinations thereof.

### Nucleic Acid Constructs or Parts

Provided herein are nucleic acid construct or parts for use in assembly methods provided herein to generate a nucleic acid construct comprising a plurality of guide RNAs (gRNAs) interspersed with tRNA sequences at regular intervals provided herein. In one embodiment, provided herein is a nucleic acid construct comprising a spacer sequence or targeting sequence flanked by restriction endonuclease sites. A nucleic acid construct comprising a spacer sequence flanked by restriction endonuclease sites can be referred to as a “spacer part”. In another embodiment, provided herein is a nucleic acid construct comprising a guide nucleic acid flanked by restriction endonuclease sites. In still another embodiment, provided herein is a nucleic acid construct comprising a guide nucleic acid and a tRNA sequence flanked by restriction endonuclease sites.

In some cases, the guide nucleic acid in any nucleic acid construct provided herein can refer to a polynucleotide comprising: (1) a guide sequence capable of hybridizing to a target sequence (referred to herein as a “targeting segment” or “spacer sequence”) and (2) a scaffold sequence capable of interacting with (either alone or in combination with a tracrRNA molecule) a nucleic acid guided nuclease as described herein (referred to herein as a “scaffold segment”). In some embodiments, the guide nucleic acids in any nucleic acid construct provided herein are transcribed within a host cell to generate RNA guide nucleic acids and, thus, can be referred to as “guide RNAs” or “gRNAs”. A nucleic acid construct comprising a guide nucleic acid flanked by restriction endonuclease sites as provided herein can be referred to as a “gRNA part”. A nucleic acid construct comprising a guide nucleic acid and a tRNA sequence flanked by restriction endonuclease sites can be referred to as a “gRNA-tRNA sequence part”. A gRNA-tRNA sequence part can comprise from 5′ to 3′, the gRNA and the tRNA sequence or the tRNA sequence and the gRNA. The guide nucleic acid or gRNA portion of a nucleic acid construct provided herein can comprise a targeting segment and a scaffold segment. In some embodiments, the scaffold segment of a gRNA is present in one nucleic acid construct, while the targeting segment is present in another separate nucleic acid construct (e.g., a nucleic acid construct comprising a guide nucleic acid alone or a guide nucleic acid and a tRNA sequence flanked by restriction endonuclease sites). Such embodiments are referred to herein as “double-molecule gRNAs” or “two-molecule gRNAs” or “dual gRNAs.” In some embodiments, the gRNA is present as a single nucleic acid construct (e.g., a nucleic acid construct comprising a guide nucleic acid alone or a guide nucleic acid and a tRNA sequence flanked by restriction endonuclease sites) and is referred to herein as a “single-guide RNA” or an “sgRNA.” The term “guide RNA” or “gRNA” is inclusive, referring both to two-molecule guide RNAs and sgRNAs.

In one embodiment, any nucleic acid part is DNA. The nucleic acid part can be generated as synthesized oligonucleotides or synthesized double-stranded (ds) DNA fragments (e.g., gBlocks). In some cases, the nucleic acid part is generated via polymerase chain reaction (PCR).

A DNA-targeting segment or spacer sequence provided herein (e.g., either alone or as part of a gRNA present in a nucleic acid construct provided herein) can comprise a nucleotide sequence that is complementary to a sequence in a target nucleic acid sequence. The target nucleic acid sequence of a spacer sequence can be referred to as the protospacer sequence. The target sequence can be present a locus in genetic element (e.g., plasmid, cosmid or chromosome) present in a host cell. As such, the targeting segment or spacer sequence can interact with a target nucleic acid in a sequence-specific manner via hybridization (i.e., base pairing), and the nucleotide sequence of the targeting segment can determine the location within the target DNA that the spacer sequence and any associated gRNA will bind. The degree of complementarity between a spacer sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences. In some embodiments, a spacer sequence is about or more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 75, or more nucleotides in length. In some embodiments, a spacer sequence is less than about 75, 50, 45, 40, 35, 30, 25, 20 nucleotides in length. In aspects, the spacer sequence is 10-30 nucleotides long. The spacer sequence can be 15-20 nucleotides in length. The spacer sequence can be 15 nucleotides in length. The spacer sequence can be 16 nucleotides in length. The spacer sequence can be 17 nucleotides in length. The spacer sequence can be 18 nucleotides in length. The spacer sequence can be 19 nucleotides in length. The spacer sequence can be 20 nucleotides in length.

The scaffold segment of a gRNA as provided herein can interact with a one or more Cas effector proteins to form a ribonucleoprotein complex (referred to herein as a CRISPR-RNP or a RNP-complex). The gRNA directs the bound polypeptide to a specific nucleotide sequence within a target nucleic acid sequence via the above-described targeting segment or spacer sequence. The scaffold segment of a guide RNA comprises two stretches of nucleotides that are complementary to one another and which form a double stranded RNA duplex. Sufficient sequence within the scaffold sequence to promote formation of a targetable nuclease complex may include a degree of complementarity along the length of two sequence regions within the scaffold sequence, such as one or two sequence regions involved in forming a secondary structure. In some cases, the one or two sequence regions are comprised or encoded on the same polynucleotide. In some cases, the one or two sequence regions are comprised or encoded on separate polynucleotides. Optimal alignment may be determined by any suitable alignment algorithm, and may further account for secondary structures, such as self-complementarity within either the one or two sequence regions. In some embodiments, the degree of complementarity between the one or two sequence regions along the length of the shorter of the two when optimally aligned is about or more than about 25%, 30%, 40%, 50%, 60%, 70%, 80%, 90%, 95%, 97.5%, 99%, or higher. In some embodiments, at least one of the two sequence regions is about or more than about 5, 6, 7, 8, 9, 10, 11, 12, 13, 14, 15, 16, 17, 18, 19, 20, 25, 30, 40, 50, or more nucleotides in length.

A scaffold sequence of a subject gRNA can comprise a secondary structure. A secondary structure can comprise a pseudoknot region or stem-loop structure. In some examples, the compatibility of a guide nucleic acid and nucleic acid guided nuclease is at least partially determined by sequence within or adjacent to the secondary structure region of the guide RNA. In some cases, binding kinetics of a guide nucleic acid to a nucleic acid guided nuclease is determined in part by secondary structures within the scaffold sequence. In some cases, binding kinetics of a guide nucleic acid to a nucleic acid guided nuclease is determined in part by nucleic acid sequence with the scaffold sequence. The nucleic acid guided nuclease can be any nucleic acid guided nuclease known in the art and/or provided herein.

A compatible scaffold sequence for a gRNA-Cas effector protein combination can be found by scanning sequences adjacent to a native Cas nuclease loci. In other words, native Cas nucleases can be encoded on a genome within proximity to a corresponding compatible guide nucleic acid or scaffold sequence.

Nucleic acid guided nucleases can be compatible with guide nucleic acids that are not found within the nucleases endogenous host. Such orthogonal guide nucleic acids can be determined by empirical testing. Orthogonal guide nucleic acids can come from different bacterial species or be synthetic or otherwise engineered to be non-naturally occurring. Orthogonal guide nucleic acids that are compatible with a common nucleic acid-guided nuclease can comprise one or more common features. Common features can include sequence outside a pseudoknot region. Common features can include a pseudoknot region. Common features can include a primary sequence or secondary structure.

A guide nucleic acid can be engineered to target a desired target sequence by altering the spacer sequence such that the spacer or guide sequence is complementary to the target sequence, thereby allowing hybridization between the guide sequence and the target sequence. A guide nucleic acid with an engineered guide sequence can be referred to as an engineered guide nucleic acid or gRNA. Engineered guide nucleic acids are often non-naturally occurring and are not found in nature.

The tRNA sequence in any nucleic acid construct comprising a guide nucleic acid and tRNA sequence flanked by restriction endonuclease sites as provided herein can be any tRNA sequence or portion thereof that can be recognized and processed (i.e., cleaved) by components of a tRNA processing system. The tRNA processing system can be endogenous to a host cell that contains the nucleic acid construct or can be exogenously introduced into said cell. In one embodiment, the tRNA sequence includes one or more of the group consisting of a pretRNA acceptor stem, a D-loop arm, a TψC-loop arm and any combination thereof. In one embodiment, the tRNA sequence includes an active site for one or more of the group consisting of RNase P, RNase Z, RNase E and any combination thereof. In another embodiment, the tRNA sequence includes an active site for one or more of the group consisting of RNase P, RNase F and RNase D. In another embodiment, the tRNA sequence includes a full pre-tRNA sequence. The pre-tRNA sequence can be selected from tRNA-ser, tRNA-gln, tRNA-lys and tRNA-gly. In one embodiment, the pre-tRNA sequence is selected from tRNA-ser, tRNA-gln, tRNA-lys and tRNA-gly from S. cerevisiae. The tRNA sequence used in any nucleic constructs provided herein can be exogenous or endogenous to the host cell into which the nucleic acid construct is introduced.

In one embodiment, any nucleic acid part provided herein further comprises one or more regulatory elements operably linked thereto. The regulatory elements can be selected from the group consisting of promoters, enhancers, transcription initiation sites, translation start sites, translation stop sites, and transcription terminators. In one embodiment, a nucleic acid part provided herein comprises a promoter operably linked thereto. In one embodiment, a nucleic acid part provided herein comprises a terminator operably linked thereto. In one embodiment, a nucleic acid part provided herein comprises a promoter and a terminator operably linked thereto. In one embodiment, a nucleic acid construct comprising a guide nucleic acid or a guide nucleic acid and tRNA sequence flanked by restriction endonuclease sites as provided herein comprises a promoter and a terminator that flank the guide nucleic acid or the guide nucleic acid and tRNA sequence and are operably linked thereto. The promoter sequence can be any promoter sequence provided herein. The promoter sequence can be a RNA polymerase II (Pol II) or RNA polymerase III (Pol III) promoter. In one embodiment, the promoter is a Pol III promoter. In one embodiment, the promoter is an SNR52 promoter. The terminator sequence can be any terminator sequence provided herein. The terminator sequence can be a Pol III terminator.

The restriction endonuclease sites present in any nucleic acid part provided herein can be any restriction endonuclease sites known in the art that are compatible for use in a scarless or seamless cloning technique such as, for example, Golden Gate cloning. In one embodiment, the restriction endonuclease sites are for Type IIs restriction endonucleases. The restriction endonuclease sites present in any nucleic acid part provided herein can be for an identical restriction endonuclease (e.g., type IIs restriction endonuclease) or different restriction endonuclease sites (e.g., type IIs restriction endonucleases). The type IIs restriction endonucleases for use with the constructs and methods provided herein can be any type IIs restriction endonuclease known in the art. The type IIs restriction endonuclease can be selected from the group consisting of AarI, BbsI, BsmAI, BsmBI and BsaI. In one embodiment, the type IIs restriction endonuclease sites present in any nucleic acid part provided herein are BsaI sites.

In one embodiment, nucleic acid construct comprising a guide nucleic acid and a tRNA sequence flanked by restriction endonuclease sites as provided herein comprises, from 5′ to 3′, a first restriction site (e.g., type IIs restriction site), the gRNA and the tRNA sequence with a second restriction site (e.g., type IIs restriction site) within the tRNA sequence. In one embodiment, the nucleic acid construct comprising a guide nucleic acid and a tRNA sequence flanked by restriction endonuclease sites as provided herein comprises, from 5′ to 3′, a first restriction site (e.g., Type IIs restriction site), a promoter sequence operably linked to the gRNA and the tRNA sequence with a second restriction site (e.g., type IIs restriction site) within the tRNA sequence. In one embodiment, the second restriction site (e.g., type IIs restriction site) within the tRNA sequence is within the 3′ end or terminus of the tRNA sequence. In one embodiment, a nucleic acid construct comprising a guide nucleic acid and a tRNA sequence flanked by restriction endonuclease sites as provided herein comprises, from 5′ to 3′, a first restriction site, a promoter sequence operably linked to the tRNA sequence, the gRNA, a terminator sequence operably linked to the gRNA and second restriction site. In each of these exemplary embodiments, the gRNA can comprise a spacer sequence solely or can be a sgRNA. In embodiments where the gRNA comprises a spacer sequence solely, a compatible scaffold sequence can be present as a separate expression cassette either as a fragment or within a vector. As described herein, the gRNA can comprise a spacer sequence comprising sequence complementary to a target sequence present at a locus in a genetic element in a cell.

Also provided herein are compositions comprising one or more nucleic acid parts provided herein. Said compositions can further comprise a vector and/or one or more enzymes. The one or more enzymes can be any enzyme(s) compatible with a scarless or seamless cloning technique such as, for example, Golden Gate cloning as well as one or more restriction endonucleases. The one or more restriction enzymes can be restriction enzymes that recognize restriction sites present in any of the nucleic acid parts and/or vector backbones present in said compositions. In one embodiment, the restriction sites are type IIs restriction sites and type IIs restriction endonucleases are present in the composition. The type IIs restriction endonuclease can be any type IIs restriction enzyme known in the art and/or provided herein. The vector provided in a composition provided herein can comprise a promoter sequence and a terminator sequence with one or more restriction sites (e.g., type IIs restriction sites) located therebetween such that assembly of the plurality of nucleic acid parts into the vector operably links the promoter and terminator to the plurality of nucleic acid parts.

In one embodiment, the plurality of nucleic acid parts in a composition provided herein is a plurality of gRNA-tRNA sequence parts alone. In one embodiment, the plurality of nucleic acid parts in a composition provided herein comprises one or a plurality of gRNA-tRNA sequence parts in combination with a spacer part or a gRNA part. The plurality of gRNA-tRNA sequence parts can be at least, at most or exactly 2, 3, 4, 5, 6, 7, 8, 9 or 10 gRNA-tRNA sequence parts. The tRNA sequence in each gRNA-tRNA sequence part as well as gRNA or spacer part if present can be different than the tRNA sequence in each other gRNA-tRNA sequence part as well as gRNA or spacer part if present in the plurality. In one embodiment, the plurality of gRNA-tRNA sequence parts comprises subsets of gRNA-tRNA sequence parts such that the tRNA sequence in each subset is identical but different than the tRNA sequence in each other spacer sequence comprising nucleic acid part present in the composition. In one embodiment, the spacer sequence in each nucleic acid part that comprises a spacer sequence present in a composition provided herein comprises sequence complementary to a target sequence that is present at a different locus in a genetic element in a host cell than the target sequence targeted by the spacer sequence in each other spacer sequence comprising nucleic acid part in the composition. Further to this embodiment, the spacer sequence comprising nucleic acid parts in the composition can target multiple different genes in the host cell. In another embodiment, the spacer sequence in each spacer sequence comprising nucleic acid part in the composition comprises sequence complementary to a target sequence at an identical locus in a genetic element in a host cell as the target sequence complementary to the spacer sequence in each other spacer sequence comprising nucleic acid part in the composition. Further to this embodiment, the spacer sequence comprising nucleic acid parts in the composition can target multiple sites within a single gene in the host cell. In yet another embodiment, the spacer sequences in a subset of spacer sequence comprising nucleic acid parts in the composition comprise sequences complementary to target sequences at an identical locus in a genetic element in a host cell and yet target a different locus in the genetic element than the target sequences that comprise sequence complementary to the spacer sequences in each other spacer sequence comprising nucleic acid part in the composition not in said subset. The genetic element can be a chromosome, plasmid or cosmid.

Each of the nucleic acid parts present in a composition provided herein can be designed to insert or assemble into a vector present in the composition in a pre-determined order or slot within the vector backbone. The order in which a nucleic acid part can be assembled into a vector backbone can be determined by the restriction sites (e.g., type IIs restriction sites) present on the nucleic acid part and vector backbone and the compatible ends generated following digestion of said restriction sites by the compatible restriction enzyme(s). The compatible restriction enzyme(s) can be present in or added to the composition comprising the nucleic acid parts.

In one embodiment, each nucleic acid part present in a composition provided herein that is designed to insert into a pre-determined slot within the vector backbone can be represented by a pool of nucleic acid parts. In this way, a composition provided herein can be used in a pooled assembly method. The spacer sequences present in any pool of nucleic acid parts can each comprise sequence complementary to a target sequence that resides within an identical locus in a genetic element within a host cell. The spacer sequences present in any pool of nucleic acid parts can each comprise sequence complementary to a target sequence that resides within a different locus in a genetic element within a host cell. The spacer sequences present in any pool of nucleic acid parts can comprise sequence complementary to a target sequence that is different than the target sequences that the spacer sequences in each nucleic acid part from each other pool are directed to.

In one embodiment, provided herein is a composition comprising: (a) a plasmid backbone comprising, from 5′ to 3′, a promoter sequence, a first type IIs restriction site, a stuffer sequence and a second type IIS restriction site; and (b) a plurality of gRNA-tRNA sequence parts, wherein each gRNA-tRNA sequence part in the plurality comprises, from 5′ to 3′, a first type IIs restriction site, a gRNA and a tRNA sequence with a second type IIs restriction site within the tRNA sequence. In one embodiment, the first and second type IIs restriction sites in the plasmid backbone allow for insertion of each gRNA-tRNA sequence part of the plurality of gRNA-tRNA sequence parts of (b) into the plasmid backbone upon cleavage. As provided herein, the gRNA can comprise a spacer sequence comprising sequence complementary to a target sequence present at a locus in a genetic element in a host cell. In one embodiment, the second type IIs restriction site in each of the plurality of gRNA-tRNA sequence parts is within the 3′ end or terminus of the tRNA sequence. In one embodiment, the tRNA sequences in each gRNA-tRNA sequence part in the plurality is different than the tRNA sequence in each other gRNA-tRNA sequence part in the plurality. Further to this embodiment, cleavage of the type IIs restriction sites with the compatible type IIs restriction enzyme (e.g., BsaI) on each of the plurality of gRNA-tRNA parts can generate compatible ends that allow for the assembly of the plurality of gRNA-tRNA sequence parts into the vector backbone in a pre-determined order. In other words, based on the compatible ends generated following digestion of the type IIs restriction sites with the cognate type IIs restriction enzyme, each gRNA-tRNA sequence part inserts into the vector backbone at a particular slot. In this way, each gRNA-tRNA sequence part of the plurality can be considered as an individual module. In one embodiment, each gRNA-tRNA sequence part in the plurality comprises a promoter sequence operably linked thereto. In another embodiment, only a subset of gRNA-tRNA sequence parts in the plurality comprises a promoter sequence operably linked thereto. Further to this embodiment, the subset of gRNA-tRNA sequence parts that comprise a promoter sequence operably linked thereto can be designed to insert or assemble into particular slots in the vector background at regular intervals. The regular intervals can be such that each promoter sequence present in nucleic acid construct assembled into the vector backbone is operably linked to and controls the expression of up to two, three, four or five gRNA-tRNA sequence parts. In one embodiment, the vector backbone further comprises a terminator sequence located downstream or 3′ to the second type IIs restriction site within the vector backbone.

In another embodiment, provided herein is a composition comprising: (a) a plasmid backbone comprising, from 5′ to 3′, a promoter sequence, a first type IIs restriction site, a stuffer sequence and a second type IIs restriction site; (b) a plurality of gRNA-tRNA sequence parts, wherein each gRNA-tRNA sequence part in the plurality comprises, from 5′ to 3′, a first type IIs restriction site, a gRNA and a tRNA sequence with a second type IIs restriction site within the tRNA sequence; and (c) a gRNA part, wherein the gRNA part comprises, from 5′ to 3′, a first type IIs restriction site, a gRNA and a second type IIs restriction site. In one embodiment, the first and second type IIS restriction sites in the plasmid backbone allow for insertion of each gRNA-tRNA sequence part of the plurality of gRNA-tRNA sequence parts of (b) and the gRNA part of (c) into the plasmid backbone upon cleavage. As provided herein, the gRNA in each gRNA-tRNA sequence part from the plurality and the gRNA part can comprise a spacer sequence comprising sequence complementary to a target sequence present at a locus in a genetic element in a host cell. In one embodiment, the second type IIs restriction site in each of the plurality of gRNA-tRNA sequence parts is within the 3′ end or terminus of the tRNA sequence. In one embodiment, the tRNA sequences in each gRNA-tRNA sequence part in the plurality is different than the tRNA sequence in each other gRNA-tRNA sequence part in the plurality. Further to this embodiment, cleavage of the type IIs restriction sites with the compatible type IIs restriction enzyme (e.g., BsaI) on each of the plurality of gRNA-tRNA parts as well as gRNA part can generate compatible ends that allow for the assembly of the plurality of gRNA-tRNA sequence parts as well as gRNA part into the vector backbone in a pre-determined order. In other words, based on the compatible ends generated following digestion of the type IIs restriction sites with the cognate type IIs restriction enzyme, each gRNA-tRNA sequence part as well as gRNA part inserts into the vector backbone at a particular slot. In this way, each gRNA-tRNA sequence part of the plurality as well as the gRNA part can be considered as an individual module. In one embodiment, the gRNA part is designed to assemble or insert into the vector backbone in the slot adjacent to or immediately upstream of the second type IIs restriction site within the vector backbone. In one embodiment, each gRNA-tRNA sequence part in the plurality comprises a promoter sequence operably linked thereto. In another embodiment, only a subset of gRNA-tRNA sequence parts in the plurality comprises a promoter sequence operably linked thereto. Further to this embodiment, the subset of gRNA-tRNA sequence parts that comprise a promoter sequence operably linked thereto can be designed to insert or assemble into particular slots in the vector background at regular intervals. The regular intervals can be such that each promoter sequence present in nucleic acid construct assembled into the vector backbone is operably linked to and controls the expression of up to two, three, four or five gRNA-tRNA sequence parts. In one embodiment, the gRNA part comprises a promoter sequence operably linked thereto. In another embodiment, the gRNA part comprises a terminator sequence operably linked thereto. In yet another embodiment, the gRNA part comprises a promoter sequence and a terminator sequence operably linked thereto. The promoter sequence in a gRNA part can be located 3′ or downstream to the first type IIs restriction site in the gRNA part. The terminator sequence in a gRNA part can be located upstream or 5′ to the second type IIs restriction site within the gRNA part. In one embodiment, the vector backbone further comprises a terminator sequence located downstream or 3′ to the second type IIs restriction site within the vector backbone.

In still another embodiment, provided herein is a composition comprising: (a) a plasmid backbone comprising, from 5′ to 3′, a promoter sequence, a first type IIs restriction site, a stuffer sequence and a second type IIs restriction site; (b) a plurality of gRNA-tRNA sequence parts, wherein each gRNA-tRNA sequence part in the plurality comprises, from 5′ to 3′, a first type IIs restriction site, a gRNA and a tRNA sequence with a second type IIs restriction site within the tRNA sequence; and (c) a spacer part, wherein the spacer part comprises, from 5′ to 3′, a first type IIs restriction site, a spacer sequence and a second type IIs restriction site. In one embodiment, the first and second type IIS restriction sites in the plasmid backbone allow for insertion of each gRNA-tRNA sequence part of the plurality of gRNA-tRNA sequence parts of (b) and the spacer part of (c) into the plasmid backbone upon cleavage. As provided herein, the gRNA in each gRNA-tRNA sequence part from the plurality and the spacer part can comprise a spacer sequence comprising sequence complementary to a target sequence present at a locus in a genetic element in a host cell. In one embodiment, the second type IIs restriction site in each of the plurality of gRNA-tRNA sequence parts is within the 3′ end or terminus of the tRNA sequence. In one embodiment, the tRNA sequences in each gRNA-tRNA sequence part in the plurality is different than the tRNA sequence in each other gRNA-tRNA sequence part in the plurality. Further to this embodiment cleavage of the type IIs restriction sites with the compatible type IIs restriction enzyme (e.g., BsaI) on each of the plurality of gRNA-tRNA parts as well as spacer part can generate compatible ends that allow for the assembly of the plurality of gRNA-tRNA sequence parts as well as spacer part into the vector backbone in a pre-determined order. In other words, based on the compatible ends generated following digestion of the type IIs restriction sites with the cognate type IIs restriction enzyme, each gRNA-tRNA sequence part as well as spacer part inserts into the vector backbone at a particular slot. In this way, each gRNA-tRNA sequence part of the plurality as well as the spacer part can be considered as an individual module. In one embodiment, the spacer part is designed to assemble or insert into the vector backbone in the slot adjacent to or immediately upstream of the second type IIs restriction site within the vector backbone. In one embodiment, each gRNA-tRNA sequence part in the plurality comprises a promoter sequence operably linked thereto. In another embodiment, only a subset of gRNA-tRNA sequence parts in the plurality comprises a promoter sequence operably linked thereto. Further to this embodiment, the subset of gRNA-tRNA sequence parts that comprise a promoter sequence operably linked thereto can be designed to insert or assemble into particular slots in the vector background at regular intervals. The regular intervals can be such that each promoter sequence present in nucleic acid construct assembled into the vector backbone is operably linked to and controls the expression of up to two, three, four or five gRNA-tRNA sequence parts. In one embodiment, the spacer part comprises a promoter sequence operably linked thereto. The promoter sequence in a spacer part can be located 3′ or downstream to the first type IIs restriction site in the spacer part. In one embodiment, the vector backbone further comprises a terminator sequence located downstream or 3′ to the second type IIs restriction site within the vector backbone. In another embodiment, the vector backbone comprises a scaffold portion of a gRNA that operably links to the spacer part upon insertion or assembly of the spacer part into the vector backbone. The scaffold portion of the gRNA in the vector backbone can be located downstream or 3′ to the second type IIs restriction site within the gRNA part but upstream or 5′ to the terminator sequence in the vector backbone, if present.

An example of the nucleic acid parts and a vector backbone that can be present in a composition provided herein for an embodiment where the composition comprises a mixture of gRNA-tRNA sequence parts and a spacer part can be found in FIGS. 1A-1B. The nucleic parts and vector present in the composition can be assembled using the assembly methods provided herein into a nucleic acid construct comprising a plurality of guide RNAs (gRNAs) interspersed with tRNA sequences at regular intervals as shown in FIG. 1C. In this example, the nucleic acid parts comprise a vector backbone (see FIG. 1A) and nucleic acid parts (see FIG. 1B). The vector backbone (i.e., plasmid backbone) comprises, from 5′ to 3′, a promoter sequence (i.e., pSNR52 promoter) followed by a first type IIs restriction site (i.e., BsaI site), a stuffer sequence, a second type IIs restriction site (i.e., BsaI site) the scaffold portion of a gRNA and a terminator sequence (i.e., the “T” in FIG. 1A). As shown in FIG. 1B, the nucleic acid parts in the composition consist of a plurality of gRNA-tRNA sequence parts and a spacer part such that the gRNA-tRNA parts comprise, from 5′ to 3′, a first type IIs restriction site (i.e., BsaI site), a gRNA, a tRNA sequence and a second type IIs restriction site (i.e., BsaI site) that is present in the 3′ terminal portion of the tRNA sequence, while the spacer part comprises, from 5′ to 3′, a first type IIs restriction site (i.e., BsaI site), a spacer sequence portion of a gRNA, and a second type IIs restriction site (i.e., BsaI site). As can be seen in FIGS. 1A-1B, the vector backbone and each nucleic acid part within the composition has been designed (via placement of the respective type IIs restriction sites) to assemble or insert into the vector backbone in a pre-determined or specified order. In particular, FIG. 1A shows a plasmid or vector backbone that comprises type IIs restriction endonuclease sites (e.g., BsaI sites) for use in creating overhangs A and D in the promoter and gRNA backbone found within the plasmid, respectively, while FIG. 1B shows nucleic acid parts (i.e., gRNA-tRNA sequence parts and spacer part) such that nucleic acid parts for slot 1 comprise a 5′ BsaI site that can create an overhang that will be complementary to overhang A generated within the SNR52 promoter in the plasmid backbone shown in FIG. 1A, while the 3′ BsaI site in slot 1 can create an overhang B in the 3′ end of tRNA-1. Further, nucleic acid parts for slot 2 comprise BsaI sites that can create 5′ overhang B and 3′ overhang C, while the nucleic acid parts that can be cloned into slot 3 or the last slot or location within the construct do not contain the gRNA backbone but rather a spacer sequence alone and have BsaI sites that can create 5′ overhang C and 3′ overhang D for ligation onto tRNA 2 and the plasmid backbone, respectively. Ultimately, through the use of a scarless cloning technique (e.g., Golden Gate (GG) cloning reaction) as provided herein, a circular plasmid as shown in FIG. 1C can be generated comprising the three (3) nucleic acid parts from FIG. 1B separated by different tRNAs assembled into the plasmid backbone shown in FIG. 1A. It should be noted that the nucleic acid parts can be assembled directly into a plasmid backbone that comprises a nucleic acid guided nuclease (e.g., a Cas9 containing plasmid), or into a separate cloning plasmid and then amplified by PCR and transformed directly into yeast as a linear piece of DNA with homology arms that comprise sequence complementary to sequence in a yeast plasmid that are used for integration into the yeast plasmid. In one embodiment, each of the spacer sequences in the construct of FIG. 1C can comprise sequence complementary to a target or protospacer sequence present at a different locus in the genome of a host cell. In this way, the construct of FIG. 1C can be used for multiplexed genetic editing using the editing methods provided herein. Importantly, while this figure demonstrates multiplexed editing of three (3) loci, one may use the approach to edit many loci simultaneously—limited only by the number of tRNAs with 3′ terminal sequences such that digestion with a type IIs restriction enzyme at a type IIs restriction site added within or at the 3′ terminus of said tRNA sequence can generate unique 3′ overhangs. The same strategy can be used to create arrays of more than three (3) gRNAs by using additional parts similar to the slot 2 part with different tRNAs. The use of different tRNA sequences within the nucleic acid construct as described herein can serve to increase the stability of the expression vector when present in a host cell as compared to similar expression vectors that contain identical tRNA sequences in an array of gRNAs interspersed with tRNA sequences.

In one embodiment, each gRNA-tRNA sequence part, gRNA part and/or spacer part present in a composition provided herein can be represented by a pool of gRNA-tRNA sequence acid parts, a pool of gRNA parts and/or a pool of spacer parts. In this way, the composition provided herein can be used in a pooled assembly method as provided herein. The spacer sequences present in each pool of parts (i.e., gRNA-tRNA sequence part, gRNA part or spacer part) can each comprise sequence complementary to a target sequence that resides within an identical locus in a genetic element within a host cell. The spacer sequences present in each pool of parts (i.e., gRNA-tRNA sequence part, gRNA part or spacer part) can each comprise sequence complementary to a target sequence that resides within a different locus in a genetic element within a host cell. The spacer sequence present in each pool of parts (i.e., gRNA-tRNA sequence part, gRNA part or spacer part) can each comprise sequence complementary to a target sequence that is different than the target sequences that the spacer sequences in each gRNA-tRNA sequence part from each other pool are directed to. The genetic element can be a chromosome, plasmid or cosmid.

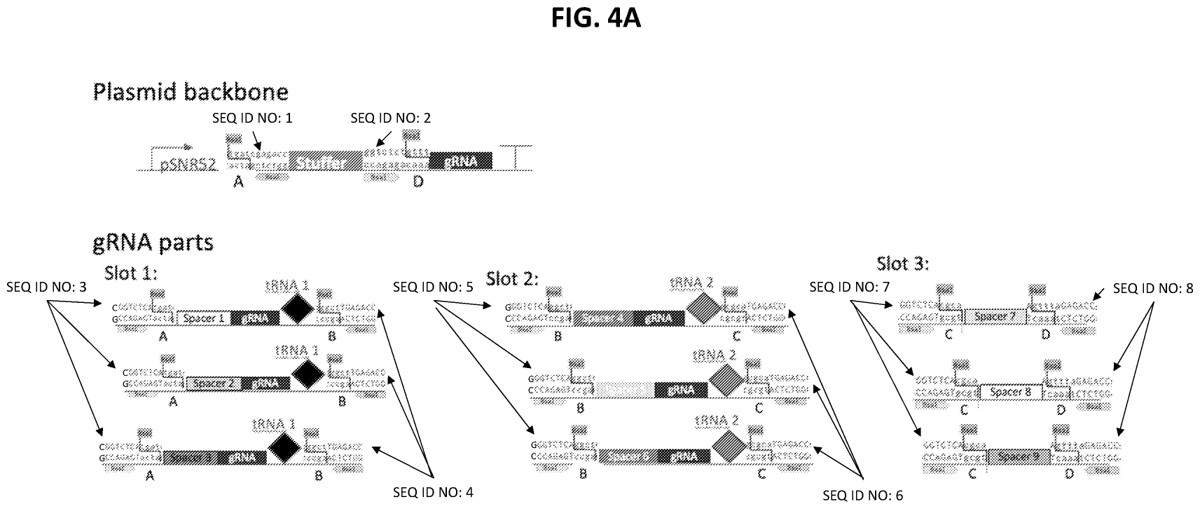

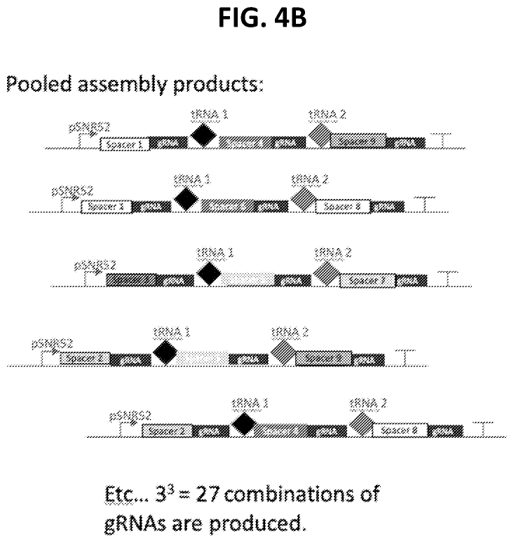

An example of an embodiment where each nucleic acid part in a composition provided herein is represented by a pool of nucleic acid parts can be found in FIGS. 4A-4B. Similar to FIGS. 1A-1B, FIG. 4A shows a vector background (i.e., plasmid backbone) comprising, from 5′ to 3′, a promoter sequence (i.e., pSNR52 promoter) followed by a first type IIs restriction site (i.e., BsaI site), a stuffer sequence, a second type IIs restriction site (i.e., BsaI site) the scaffold portion of a gRNA and a terminator sequence (i.e., the “T” in FIG. 1A) as well as a plurality of gRNA-tRNA sequence parts and a spacer part such that the gRNA-tRNA parts comprise, from 5′ to 3′, a first type IIs restriction site (i.e., BsaI site), a gRNA, a tRNA sequence and a second type IIs restriction site (i.e., BsaI site) that is present in the 3′ terminal portion of the tRNA sequence, while the spacer part comprises, from 5′ to 3′, a first type IIs restriction site (i.e., BsaI site), a spacer sequence portion of a gRNA, and a second type IIs restriction site (i.e., BsaI site). Further, like FIG. 1B, the vector backbone and each nucleic acid part in FIG. 4B has been designed (via placement of the respective type IIs restriction sites) to assemble or insert into the vector backbone in a pre-determined or specified order; however, unlike FIG. 1B, FIG. 4A shows that the nucleic acid parts designed to assemble or insert into a particular slot within the vector backbone is represented by a pool of nucleic parts (i.e., either gRNA-tRNA sequence parts or spacer parts) such that each member of a respective pool possesses an identical tRNA sequence where applicable (i.e., in gRNA-tRNA sequence parts), but a different spacer sequence. Assembly of the pools of nucleic acid parts into the vector backbone using a scarless cloning technique (e.g., GG cloning) can yield a library of nucleic acid constructs comprising gRNA sequences interspersed with tRNA sequences at regular intervals that can comprise numerous combinations of the nucleic acid parts from the respective nucleic acid part pools as shown in FIG. 4B. For example, as shown in FIG. 4B, three (3) pools of nucleic acid parts each comprising three (3) parts per pool for insertion into three (3) slots in a vector backbone can yield a library comprising potentially twenty-seven (27) combinations (i.e., 33 combinations) of multiple gRNA containing constructs.

The promoter sequence present in the vector backbone, gRNA-tRNA sequence part, spacer sequence part and/or gRNA part can be a Pol III promoter such as, for example, an SNR52 gene promoter (i.e., pSNR52). The terminator sequence present in the vector backbone, gRNA-tRNA sequence part, spacer sequence part and/or gRNA part can be a Pol III terminator sequence. In some cases, the composition further comprises a type IIs restriction enzyme that recognizes the first and/or the second type IIs restriction sites in the plasmid backbone and each gRNA-tRNA sequence part from the plurality. In one embodiment, the type IIs restriction enzyme is BsaI.

The tRNA sequence in any gRNA-tRNA sequence part present in a composition provided herein can be any tRNA sequence or portion thereof that can be recognized and processed (i.e., cleaved) by components of a tRNA processing system. The tRNA processing system can be endogenous to a host cell that contains the nucleic acid constructs or can be exogenously introduced into said cell. In one embodiment, the tRNA sequence comprises one or more of the group consisting of a pretRNA acceptor stem, a D-loop arm, a TψC-loop arm and any combination thereof. In one embodiment, the tRNA sequence comprises an active site for one or more of the group consisting of RNase P, RNase Z, RNase E and any combination thereof. In another embodiment, the tRNA sequence includes an active site for one or more of the group consisting of RNase P, RNase F and RNase D. In another embodiment, the tRNA sequence comprises a full pre-tRNA sequence. The pre-tRNA sequence can be selected from tRNA-ser, tRNA-gln, tRNA-lys and tRNA-gly. In one embodiment, the pre-tRNA sequence is selected from tRNA-ser, tRNA-gln, tRNA-lys and tRNA-gly from S. cerevisiae. The tRNA sequences used in the nucleic constructs provided herein can be exogenous or endogenous to the host cell into which the nucleic acid construct is introduced.

### Assembly Methods

Also provided herein are assembly methods for inserting nucleic acids parts provided herein into vector backbones in order to generate assembled vectors or plasmids comprising a plurality of guide RNAs (gRNAs) interspersed with tRNA sequences at regular intervals. Said assembled vectors or plasmids can subsequently be used in gene editing methods to generate genetically engineered organisms.

In one embodiment, provided herein is a method for preparing a vector (e.g., plasmid) comprising an array of guide RNA (gRNA)-tRNA sequences in an ordered arrangement, the method comprising: incubating under conditions that allow for digestion, a composition comprising: (i) a type IIs restriction enzyme, (ii) a vector backbone comprising, from 5′ to 3′, a promoter sequence, a first type IIs restriction site, a stuffer sequence and a second type IIs restriction site, wherein the first and the second Type IIs restriction site are recognized and digested by the type IIs restriction enzyme of (i) and (iii) a plurality of gRNA-tRNA sequence parts, wherein each gRNA-tRNA part in the plurality comprises, from 5′ to 3′, a first type IIs restriction site, a gRNA, a tRNA sequence and a second type IIs restriction site, wherein the first and the second Type IIs restriction site are recognized and digested by the type IIs restriction enzyme of (i), wherein digestion of the vector backbone and plurality of gRNA-tRNA sequence parts generates compatible ends (e.g., overhang sequences) on the vector backbone and each of the plurality of gRNA-tRNA sequence parts that allow each of the plurality of gRNA-tRNA sequence parts to insert into the vector backbone in a pre-determined order or series. In this way, each gRNA-tRNA sequence part can be said to be designed to insert into a specific slot within the vector backbone. The type IIs restriction site in each gRNA-tRNA sequence part in the plurality can be present within the tRNA sequence. In one embodiment, the second type IIs restriction site is present in the 3′ end or terminus of the tRNA sequence. Following digestion of the plasmid backbone and plurality of gRNA-tRNA sequence parts, the method can further comprise incubating the mixture with a ligase under conditions that allow for hybridization and covalent joining of the compatible ends generated in the composition. The ligase can be any ligase known in the art. In one embodiment, the ligase is a ligase compatible with a scarless cloning method such as Golden Gate cloning. Ligation can serve to insert the plurality of gRNA-tRNA sequence parts into the vector backbone in the pre-determined order, thereby generating an assembled vector backbone comprising each of the plurality of gRNA-tRNA sequence parts in a defined order. In some cases, the plurality of gRNA-tRNA sequence parts are arranged tandemly. The assembly method provided herein can further comprise (c) propagating the assembled vector in a microbial cell; and (d) isolating nucleic acid (e.g., plasmid nucleic acid that comprises the assembled plasmid) from the microbial cell(s) of step (c). The propagating step can entail introducing the assembly vector into the microbial cell and growing the microbial cell in growth media. The growth media can comprise one or more selection agents as needed. The introduction can be via any method for inserting nucleic acid into a microbial cell known in the art and/or provided herein. In one embodiment, the introduction of the assembled vector entails transformation. The transforming can be performed using any transformation method known in the art and/or provided herein that is compatible with the microbial cell selected for propagation. In one embodiment, the microbial cell is a strain of E. coli. The isolating can comprise selecting or picking transformants from step (c) and extracting and/or purifying nucleic acid isolated from the selected transformants. In some cases, individual transformants are selected or picked followed by extraction or purification of nucleic acid from said individual transformants. In some cases, transformants are gathered en masse followed by extraction or purification of nucleic acid from said transformants. In some cases, the nucleic acid extracted or purified is plasmid nucleic acid that contains or comprises the propagated assembled vector(s). The assembly method can further comprise sequencing the nucleic acid (e.g., plasmid nucleic acid including the assembled vector) from step (d) in order to confirm the sequence of the order series of gRNA-tRNA sequence parts within the assembled vector. The sequencing can be any sequencing method known in the art such as, for example, a next generation sequencing method. In one embodiment, the isolated assembled vector can be introduced directly into a host cell in order to facilitate gene editing as provided herein or the assembled vector can be linearized and subsequently introduced in the host cell. The introduction can be via transformation as provided herein.

In one embodiment, one of the gRNA-tRNA sequence parts in the plurality of gRNA-tRNA sequence parts for use in an assembly method provided herein is a spacer part as provided herein or a gRNA part as provided herein. If present, the spacer part or gRNA part can be designed to assemble or insert into the vector backbone in the slot adjacent to or immediately upstream of the second type IIs restriction site within the vector backbone. The plurality of gRNA-tRNA sequence parts can be at least, at most or exactly 2, 3, 4, 5, 6, 7, 8, 9 or 10 gRNA-tRNA sequence parts. The tRNA sequence in each gRNA-tRNA sequence part as well as gRNA or spacer part if present can be different than the tRNA sequence in each other gRNA-tRNA sequence part as well as gRNA or spacer part if present in the composition. In one embodiment, the plurality of gRNA-tRNA sequence parts comprises subsets of gRNA-tRNA sequence parts such that the tRNA sequence in each subset is identical but different than the tRNA sequence in each other spacer sequence comprising nucleic acid part present in the composition. In one embodiment, the spacer sequence in each nucleic acid part that comprises a spacer sequence present in a composition provided herein comprises sequence complementary to a target sequence that is present at a different locus in a genetic element in a host cell than the target sequence targeted by the spacer sequence in each other spacer sequence comprising nucleic acid part in the composition. Further to this embodiment, the spacer sequence comprising nucleic acid parts in the composition can target multiple different genes in the host cell. In another embodiment, the spacer sequence in each spacer sequence comprising nucleic acid part in the composition comprises sequence complementary to a target sequence at an identical locus in a genetic element in a host cell as the target sequence complementary to the spacer sequence in each other spacer sequence comprising nucleic acid part in the composition. Further to this embodiment, the spacer sequence comprising nucleic acid parts in the composition can target multiple sites within a single gene in the host cell. In yet another embodiment, the spacer sequences in a subset of spacer sequence comprising nucleic acid parts in the composition comprise sequences complementary to target sequences at an identical locus in a genetic element in a host cell and yet target a different locus in the genetic element than the target sequences that comprise sequence complementary to the spacer sequences in each other spacer sequence comprising nucleic acid part in the composition not in said subset. The genetic element can be a chromosome, plasmid or cosmid.

The vector backbone for use in an assembly method provided herein can comprise a promoter sequence and/or a terminator sequence. In some cases, the ligating step can serve to operably link the promoter and/or terminator to the plurality of gRNA-tRNA sequence parts. In one embodiment, each gRNA-tRNA sequence part in the plurality comprises a promoter sequence operably linked thereto. In another embodiment, only a subset of gRNA-tRNA sequence parts in the plurality comprises a promoter sequence operably linked thereto. Further to this embodiment, the subset of gRNA-tRNA sequence parts that comprise a promoter sequence operably linked thereto can be designed to insert or assemble into particular slots in the vector background at regular intervals. The regular intervals can be such that each promoter sequence present in nucleic acid construct assembled into the vector backbone is operably linked to and controls the expression of up to two, three, four or five gRNA-tRNA sequence parts. If present, a spacer part or gRNA part can comprise a promoter sequence operably linked thereto. The promoter sequence in a spacer part or gRNA part can be located 3′ or downstream to the first type IIs restriction site in the spacer part. If present, a gRNA part can comprise a terminator sequence. The terminator sequence in a gRNA part can be located 5′ or upstream of the second type IIs restriction site in the gRNA part. In embodiments that utilize a spacer part, the vector backbone further comprises a scaffold comprising sequence necessary to bind to the RNA-guided DNA endonuclease (e.g., Cas9) 3′ to the second Type IIS restriction site. The ligating step can serve to operably link the spacer part to the scaffold sequence present in the vector backbone.

In one embodiment, provided herein is a method for preparing a plasmid comprising an array of guide RNA (gRNA)-tRNA sequences in tandem arrangement, the method comprising: (a) incubating under conditions that allow for digestion, a mixture comprising: (i) a type IIs restriction enzyme, (ii) a plasmid backbone comprising, from 5′ to 3′, a promoter sequence, a first type IIS restriction site, a stuffer sequence and a second type IIs restriction site, wherein the first and the second type IIs restriction site are recognized and digested by the type IIs restriction enzyme of (i), and (iii) a plurality of gRNA-tRNA sequence parts, wherein each gRNA-tRNA sequence part in the plurality, comprises, from 5′ to 3′, a first type IIs restriction site, a gRNA and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality, and wherein the first and the second Type IIs restriction site in each gRNA-tRNA sequence part in the plurality are recognized and digested by the Type IIs restriction enzyme of (i), wherein digestion of the plasmid backbone of (i) removes the stuffer sequence and generates a first end within the promoter sequence and a second end distal to the first end, while digestion of each gRNA-tRNA sequence part within the plurality generates opposing first and second ends on each gRNA-tRNA sequence part that are different than the opposing first and second ends on each other gRNA-tRNA sequence part in the plurality, and wherein digestion of one gRNA-tRNA sequence part from the plurality generates a first end comprising sequence complementary to the first end of the plasmid backbone, while digestion for each other gRNA-tRNA sequence part from the plurality generates a first end that comprises sequence complementary to the second end of a gRNA-tRNA cleavage sequence part from each other gRNA-tRNA sequence part, and wherein digestion of one gRNA-tRNA sequence part from the plurality generates a second end comprising sequence complementary to the second end of the plasmid backbone; and (b) incubating the mixture with a ligase under conditions that allow for hybridization and covalent joining of first and/or second ends that comprise complementary sequence present within the mixture, wherein ligation operably links the promoter sequence in the plasmid backbone to an array of gRNA-tRNA sequence parts from the plurality of gRNA-tRNA sequence parts in tandem arrangement, thereby generating an assembled plasmid comprising an array of guide RNA (gRNA)-tRNA sequences in tandem arrangement. In one embodiment, the plasmid backbone further comprises a scaffold comprising sequence necessary to bind to an RNA-guided DNA endonuclease (e.g., Cas9) 3′ to the second Type IIs restriction site. Further to this embodiment, the one gRNA-tRNA sequence part from the plurality whose second end comprises sequence complementary to the second end of the plasmid backbone following digestion with the type IIs restriction enzyme of (i) differs from each other gRNA-tRNA sequence part from the plurality in that said gRNA-tRNA sequence part is a spacer part that comprises, from 5′ to 3′, the first Type IIs restriction site, a spacer sequence and the second Type IIs restriction site.

FIGS. 1A-1C depict the parts (FIGS. 1A-1B) that can be utilized in and the product (FIG. 1C) that can be generated by the assembly method described by the above embodiment. In particular, FIGS. 1A-1C show three (3) nucleic acid parts that can be inserted into a vector backbone in a specific order that is governed by the location of type IIs restriction sites within the parts and vector backbone. FIG. 1A shows a plasmid or vector backbone that contains type IIs BsaI sites such that digestion with BsaI creates overhangs A and D in the promoter and gRNA backbone found within the plasmid, respectively. FIG. 1B shows nucleic acid parts (i.e., gRNA-tRNA sequence parts and spacer part) that can be assembled into locations or slots within the vector background in a modular fashion as provided herein. Parts for slot 1 comprise a 5′ BsaI site that upon digestion with BsaI creates an overhang that is complementary to overhang A generated within the SNR52 promoter (pSNR52) in the plasmid backbone shown in FIG. 1A, while the 3′ BsaI site in slot 1 upon digestion with BsaI creates an overhang B in the 3′ end of tRNA-1. Parts for slot 2 have BsaI sites that create 5′ overhang B and 3′ overhang C when digested with BsaI. The gRNA-tRNA parts that can be cloned into slot 3 or the last slot or location within the backbone do not contain the gRNA backbone and have BsaI sites that can create 5′ overhang C and 3′ overhang D upon digestion with BsaI for ligation onto tRNA 2 and the plasmid backbone, respectively. Following digestion, a ligase can then be used to create a circular plasmid as shown in FIG. 1C.

In one embodiment, an assembly method provided herein is a pooled assembly method. The pooled assembly method can be used to generate a library of assembled plasmids comprising an array of guide RNA (gRNA)-tRNA sequences in tandem arrangement. Further to this embodiment, each gRNA-tRNA sequence part present in a composition provided herein that is designed to insert into a pre-determined slot within the vector backbone can be represented by a pool of gRNA-tRNA sequence parts for that particular spot. The spacer sequences present in any pool of gRNA-tRNA sequence parts designed to assemble into any particular slot in the vector backbone can each comprise sequence complementary to a target sequence that resides within an identical locus in a genetic element within a host cell. The spacer sequences present in any pool of gRNA-tRNA sequence parts designed to assemble into any particular slot in the vector backbone can each comprise sequence complementary to a target sequence that resides within a different locus in a genetic element within a host cell. The spacer sequences present in any pool of gRNA-tRNA sequence parts designed to assemble into any particular slot in the vector backbone can comprise sequence complementary to a target sequence that is different than the target sequences that the spacer sequences in each gRNA-tRNA sequence part from each other pool are directed to. As described previously, in some cases, one of the plurality of gRNA-tRNA sequence parts in the plurality of gRNA-tRNA sequence parts for use in an assembly method provided herein can actually be a spacer part as provided herein or a gRNA part as provided herein. In cases where a spacer part or gRNA part is present, said spacer part or gRNA part can be represented by a pool of spacer parts or a pool of gRNA parts. The genetic element can be a chromosome, plasmid or cosmid.

FIGS. 4A-4B provide an example of how an assembly method using the parts shown in FIGS. 1A-1B can be pooled in nature. In particular, FIG. 4A illustrates an embodiment where each of the gRNA-tRNA sequence parts from FIG. 1B that are designed to insert into a particular slot in the plasmid backbone is represented by a pool of gRNA-tRNA sequence parts for each particular slot. Following digestion with the appropriate type IIs restriction enzyme (e.g., BsaI) and subsequent assembly using a ligase in a scarless cloning method (e.g., Golden Gate cloning), a library of nucleic acid constructs comprising gRNA-tRNA sequence parts arranged in tandem is created as shown in FIG. 4B.

The promoter sequence present in the vector backbone, gRNA-tRNA sequence part, spacer sequence part and/or gRNA part can be a Pol III promoter such as, for example, an SNR52 gene promoter (i.e., pSNR52). The terminator sequence present in the vector backbone, gRNA-tRNA sequence part, spacer sequence part and/or gRNA part can be a Pol III terminator sequence.

The tRNA sequence in any gRNA-tRNA sequence part present in a composition for use in an assembly method provided herein can be any tRNA sequence or portion thereof that can be recognized and processed (i.e., cleaved) by components of a tRNA processing system. The tRNA processing system can be endogenous to a host cell that contains the nucleic acid constructs or can be exogenously introduced into said cell. In one embodiment, the tRNA sequence comprises one or more of the group consisting of a pretRNA acceptor stem, a D-loop arm and a TψC-loop arm. In one embodiment, the tRNA sequence comprises an active site for one or more of the group consisting of RNase P, RNase Z and RNase E. In another embodiment, the tRNA sequence includes an active site for one or more of the group consisting of RNase P, RNase F and RNase D. In another embodiment, the tRNA sequence comprises a full pre-tRNA sequence. The pre-tRNA sequence can be selected from tRNA-ser, tRNA-gln, tRNA-lys and tRNA-gly. In one embodiment, the pre-tRNA sequence is selected from tRNA-ser, tRNA-gln, tRNA-lys and tRNA-gly from S. cerevisiae. The tRNA sequences used in the nucleic constructs provided herein can be exogenous or endogenous to the host cell into which the nucleic acid construct is introduced.

### Gene Editing

In one aspect, provided herein is a method for modifying a genetic element (e.g., genome, cosmid, or plasmid) within a host cell using a system that uses a nucleic acid guided nuclease such as, for example, CRISPR. In one embodiment, provided herein is a method for editing the genome of a host cell that comprises (a) introducing a vector comprising an array of guide RNA (gRNA)-tRNA sequences in an order arrangement generated using an assembly method provided herein into a host cell expressing a nucleic acid-guided nuclease; and (b) introducing one or a plurality of repair fragments in the host cell under conditions that allow for homology-directed repair (HDR) utilizing the nucleic acid-guided nuclease. In one embodiment, the host cell utilizes the tRNA sequence in each gRNA-tRNA sequence in the array to release each gRNA from the array of gRNA-tRNA sequences in tandem arrangement. In order to utilize the tRNA sequences present in the vector introduced into the host cell, said host cell can use one or more enzyme of a tRNA processing system in order to bind and cleave the tRNA sequences, thereby releasing each gRNA from any tRNA sequence tethered thereto. The one or more enzymes can be selected from RNase P, RNase Z, RNase E and any combination thereof. The one or more enzymes can be selected from RNase P, RNase F, RNase D and any combination thereof. The one or more enzymes can be part of the endogenous tRNA processing system of the host cell or can be exogenous to the host cell. In one embodiment, the one or more enzymes of the tRNA processing system are introduced heterologously. The host cell can be any host cell provided herein. In one embodiment, the host cell is a eukaryotic cell. The eukaryotic cell can be a yeast cell or a filamentous fungal cell. The yeast cell can be Saccharomyces cerevisiae. The filamentous fungus can be Aspergillus niger. In one embodiment, the host cell is a prokaryotic cell. The prokaryotic host cell can be Escherichia coli or Corynebacterium glutamicum. In one embodiment, the vector is an assembled plasmid. The vector (e.g., assembled plasmid) comprising an array of guide RNA (gRNA)-tRNA sequences in an ordered arrangement can further comprise a selectable marker gene. The selectable marker gene can be any selectable marker gene known in the art and/or provided herein such as, for example an antibiotic resistance gene, an auxotrophic marker, a colorimetric marker, and a directional marker. The vector (e.g. assembled plasmid) comprising an array of guide RNA (gRNA)-tRNA sequences in an ordered arrangement can further comprise a centromere and/or autonomously replicating sequence. The vector (e.g., assembled plasmid) comprising an array of gRNA-tRNA sequence in an ordered arrangement can be removed as a terminal step in a gene editing method provided herein. Removal of the vector (e.g., assembled plasmid) can be accomplished by a passive counterselection method that entails growing host cells containing the vector in media not selective for a selectable marker gene present in the vector. Removal of the vector (e.g., assembled plasmid) can also be accomplished via CRISPR. The CRISPR based removal can entail introducing into the host cell containing the vector one or more gRNAs that comprise sequence complementary or homologous to target sequences present on the vector, whereby an RNA-guided nuclease complexes with the one or more gRNAs and cleaves the vector at the target sequences. Removal of the vector (e.g., assembled plasmid) can be accomplished by making sure the vector is a temperate sensitive replicon such that the vector loses stability when the host cell is present in a medium whose temperature is outside of a defined temperature or temperature range.

In one embodiment, each repair fragment in the plurality of repair fragments comprises a repair fragment for a gRNA released in step (a) of the method. Each repair fragment can be used in combination with a gRNA in a CRISPR method of gene editing using homology directed repair (HDR) such that a CRISPR-gRNA complex can form following release of each gRNA from the ordered array introduced into the host cell. Each repair fragment can comprise at least one desired genetic perturbation or edit (e.g., a substitution, an inversion, an insertion, a deletion, a single nucleotide polymorphism, and any combination thereof) as well as targeting sequences derived from homology arms present on opposing ends of the repair fragment that comprise sequence complementary to the locus targeted by the gRNA. In this embodiment, the CRISPR-gRNA complex cleaves the target gene specified by the one or more gRNAs. The repair fragment can then be used as a template for the homologous recombination machinery to incorporate the desired genetic perturbation or edit into the host cell. The repair fragment can be single-stranded, double-stranded or a double-stranded plasmid. In one embodiment, each repair fragment from the plurality of repair fragments is provided on a plasmid or as a linear fragment. In another embodiment, each repair fragment from the plurality of repair fragments is provided as a ssDNA or dsDNA linear fragment. The repair fragment can lack a PAM sequence or comprise a scrambled, altered or non-functional PAM in order to prevent re-cleavage. In some cases, the repair fragment can contain a functional or non-altered PAM site. The mutated or edited sequence in the repair fragment (also flanked by the regions of homology) can prevents re-cleavage by the CRISPR-complex after the genetic edit(s) has/have been incorporated into the genome.

In one embodiment, each repair fragment from the plurality of repair fragments is represented by a pool of repair fragments. The at least one genetic edit within each repair fragment in a respective pool of repair fragments can be different than the at least one genetic edit in each other repair fragment in the respective pool of repair fragments.

A genetic edit for use in a gene editing method provided herein can be a random sequence. A genetic edit can be a marker sequence. The marker sequence can be any marker sequence known in the art. A genetic edit can be a gene or a portion thereof. The gene or portion thereof can be part of a metabolic or biochemical pathway. The gene or portion thereof can encode a protein or a domain thereof. A genetic edit can be whole or portions of promoters, genes, regulatory sequences, nucleic acid sequence encoding degrons, nucleic acid sequence encoding solubility tags, nucleic acid sequence encoding degradation tags, terminators, barcodes, regulatory sequences or portions thereof. In some cases, one or each of the at least one genetic edit on a repair fragment can comprise a barcode sequence. The barcode sequence can comprise a sequence unique to each combination of repair fragments and homology arms flanked by sequence universal to the barcode sequence present in each other repair fragment. The sequence universal to the barcode sequence present in each other repair fragment can be used for amplifying or sequencing the unique sequence in each barcode. The marker sequence can be any selectable marker sequence known in the art. The selectable marker sequence can be selected from the group consisting of an antibiotic resistance gene, an auxotrophic marker, a colorimetric marker, and a directional marker. In one embodiment, the at least one genetic edit present in a repair fragment provided herein is selected from SNP swaps, Promoter swaps, ORF insertions, pathway insertions and deletions and combinations thereof. In another embodiment, the at least one genetic edit present in a repair fragment provided herein is selected from whole or portions of promoters, genes, regulatory sequences, nucleic acid sequence encoding degrons, nucleic acid sequence encoding solubility tags, terminators, unique identifier sequence, and combinations thereof.

In one embodiment, the nucleic acid guided nuclease used by the host cell for use with any construct, composition and/or gene editing method provided herein is an RNA-guided DNA endonuclease. The nucleic acid guided nuclease (e.g., RNA-guided DNA endonuclease) can be encoded on a plasmid, encoded in the genome of the host cell, translated from RNA, or introduced into the host cell as protein. The RNA-guided DNA endonuclease can be part of CRISPR/Cas system.

The CRISPR/Cas system is a prokaryotic immune system that confers resistance to foreign genetic elements such as those present within plasmids and phages and that provides a form of acquired immunity. CRISPR stands for Clustered Regularly Interspaced Short Palindromic Repeat, and cas stands for CRISPR-associated system, and refers to the small cas genes associated with the CRISPR complex.

CRISPR-Cas systems are most broadly characterized as either Class 1 or Class 2 systems. The main distinguishing feature between these two systems is the nature of the Cas-effector module. Class 1 systems require assembly of multiple Cas proteins in a complex (referred to as a “Cascade complex”) to mediate interference, while Class 2 systems use a large single Cas enzyme to mediate interference. Each of the Class 1 and Class 2 systems are further divided into multiple CRISPR-Cas types based on the presence of a specific Cas protein. For example, the Class 1 system is divided into the following three types: Type I systems, which contain the Cas3 protein; Type III systems, which contain the Cas10 protein; and the putative Type IV systems, which contain the Csf1 protein, a Cas8-like protein. Class 2 systems are generally less common than Class 1 systems and are further divided into the following three types: Type II systems, which contain the Cas9 protein; Type V systems, which contain Cas12a protein (previously known as Cpf1, and referred to as Cpf1 herein), Cas12b (previously known as C2c1), Cas12c (previously known as C2c3), Cas12d (previously known as CasY), and Cas12e (previously known as CasX); and Type VI systems, which contain Cas13a (previously known as C2c2), Cas13b, and Cas13c. Pyzocha et al., ACS Chemical Biology, Vol. 13 (2), pgs. 347-356. In one embodiment, the CRISPR-Cas system for use in the methods provided herein is a Class 2 system. In one embodiment, the CRISPR-Cas system for use in the methods provided herein is a Type II, Type V or Type VI Class 2 system. In one embodiment, the CRISPR-Cas system for use in the methods provided herein is selected from Cas9, Cas12a, Cas12b, Cas12c, Cas12d, Cas12e, Cas13a, Cas13b, Cas13c or homologs, orthologs or paralogs thereof.

CRISPR systems used in methods disclosed herein comprise a Cas effector module comprising one or more nucleic acid guided CRISPR-associated (Cas) nucleases, referred to herein as Cas effector proteins. In some embodiments, the Cas proteins can comprise one or multiple nuclease domains. A Cas effector protein can target single stranded or double stranded nucleic acid molecules (e.g. DNA or RNA nucleic acids) and can generate double strand or single strand breaks. In some embodiments, the Cas effector proteins are wild-type or naturally occurring Cas proteins. In some embodiments, the Cas effector proteins are mutant Cas proteins, wherein one or more mutations, insertions, or deletions are made in a WT or naturally occurring Cas protein (e.g., a parental Cas protein) to produce a Cas protein with one or more altered characteristics compared to the parental Cas protein.

In some instances, the Cas protein is a wild-type (WT) nuclease. Non-limiting examples of suitable Cas proteins for use in the present disclosure include C2c1, C2c2, C2c3, Cas1, Cas1B, Cas2, Cas3, Cas4, Cas5, Cas6, Cas7, Cas8, Cas9 (also known as Csn1 and Csx12), Cas10, Cpf1, Csy1, Csy2, Csy3, Cse1, Cse2, Csc1, Csc2, Csa5, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx100, Csx16, CsaX, Csx3, Csx1, Csx15, Csf1, Csf2, Csf3, Csf4, MAD1-20, SmCsm1, homologues thereof, orthologues thereof, variants thereof, mutants thereof, or modified versions thereof. Suitable nucleic acid guided nucleases (e.g., Cas 9) can be from an organism from a genus, which includes but is not limited to: Thiomicrospira, Succinivibrio, Candidatus, Porphyromonas, Acidomonococcus, Prevotella, Smithella, Moraxella, Synergistes, Francisella, Leptospira, Catenibacterium, Kandleria, Clostridium, Dorea, Coprococcus, Enterococcus, Fructobacillus, Weissella, Pediococcus, Corynebacter, Sutterella, Legionella, Treponema, Roseburia, Filifactor, Eubacterium, Streptococcus, Lactobacillus, Mycoplasma, Bacteroides, Flaviivola, Flavobacterium, Sphaerochaeta, Azospirillum, Gluconacetobacter, Neisseria, Roseburia, Parvibaculum, Staphylococcus, Nitratifractor, Mycoplasma, Alicyclobacillus, Brevibacilus, Bacillus, Bacteroidetes, Brevibacilus, Carnobacterium, Clostridiaridium, Clostridium, Desulfonatronum, Desulfovibrio, Helcococcus, Leptotrichia, Listeria, Methanomethyophilus, Methylobacterium, Opitutaceae, Paludibacter, Rhodobacter, Sphaerochaeta, Tuberibacillus, and Campylobacter. Species of organism of such a genus can be as otherwise herein discussed.

Suitable nucleic acid guided nucleases (e.g., Cas9) can be from an organism from a phylum, which includes but is not limited to: Firmicute, Actinobacteria, Bacteroidetes, Proteobacteria, Spirochetes, and Tenericutes. Suitable nucleic acid guided nucleases can be from an organism from a class, which includes but is not limited to: Erysipelotrichia, Clostridia, Bacilli, Actinobacteria, Bacteroidetes, Flavobacteria, Alphaproteobacteria, Betaproteobacteria, Gammaproteobacteria, Deltaproteobacteria, Epsilonproteobacteria, Spirochaetes, and Mollicutes. Suitable nucleic acid guided nucleases can be from an organism from an order, which includes but is not limited to: Clostridiales, Lactobacillales, Actinomycetales, Bacteroidales, Flavobacteriales, Rhizobiales, Rhodospirillales, Burkholderiales, Neisseriales, Legionellales, Nautiliales, Campylobacterales, Spirochaetales, Mycoplasmatales, and Thiotrichales. Suitable nucleic acid guided nucleases can be from an organism from within a family, which includes but is not limited to: Lachnospiraceae, Enterococcaceae, Leuconostocaceae, Lactobacillaceae, Streptococcaceae, Peptostreptococcaceae, Staphylococcaceae, Eubacteriaceae, Corynebacterineae, Bacteroidaceae, Flavobacterium, Cryomoorphaceae, Rhodobiaceae, Rhodospirillaceae, Acetobacteraceae, Sutterellaceae, Neisseriaceae, Legionellaceae, Nautiliaceae, Campylobacteraceae, Spirochaetaceae, Mycoplasmataceae, and Francisellaceae.

Other nucleic acid guided nucleases (e.g., Cas9) suitable for use in the methods, systems, constructs and compositions of the present disclosure include those derived from an organism such as, but not limited to: Thiomicrospira sp. XS5, Eubacterium rectale, Succinivibrio dextrinosolvens, Candidatus Methanoplasma termitum, Candidatus Methanomethylophilus alvus, Porphyromonas crevioricanis, Flavobacterium branchiophilum, Acidomonococcus sp., Lachnospiraceae bacterium COE1, Prevotella brevis ATCC 19188, Smithella sp. SCADC, Moraxella bovoculi, Synergistes jonesii, Bacteroidetes oral taxon 274, Francisella tularensis, Leptospira inadai serovar Lyme str. 10, Acidomonococcus sp. crystal structure (5B43) S. mutans, S. agalactiae, S. equisimilis, S. sanguinis, S. pneumonia; C. jejuni, C. coli; N. salsuginis, N. tergarcus; S. auricularis, S. carnosus; N. meningitides, N gonorrhoeae; L. monocytogenes, L. ivanovii; C. botulinum, C. difficile, C. tetani, C. sordellii; Francisella tularensis 1, Prevotella albensis, Lachnospiraceae bacterium MC2017 1, Butyrivibrio proteoclasticus, Peregrinibacteria bacterium GW2011_GWA2_33_10, Parcubacteria bacterium GW2011_GWC2_44_17, Smithella sp. SCADC, Microgenomates, Acidaminococcus sp. BV3L6, Lachnospiraceae bacterium MA2020, Candidatus methanoplasma termitum, Eubacterium eligens, Moraxella bovoculi 237, Leptospira inadai, Lachnospiraceae bacterium ND2006, Porphyromonas crevioricanis 3, Prevotella disiens, Porphyromonas macacae, Catenibacterium sp. CAG:290, Kandleria vitulina, Clostridiales bacterium KA00274, Lachnospiraceae bacterium 3-2, Dorea longicatena, Coprococcus catus GD/7, Enterococcus columbae DSM 7374, Fructobacillus sp. EFB-N1, Weissella halotolerans, Pediococcus acidilactici, Lactobacillus curvatus, Streptococcus pyogenes, Lactobacillus versmoldensis, and Filifactor alocis ATCC 35896. See, U.S. Pat. Nos. 8,697,359; 8,771,945; 8,795,965; 8,865,406; 8,871,445; 8,889,356; 8,895,308; 8,906,616; 8,932,814; 8,945,839; 8,993,233; 8,999,641; 9,822,372; 9,840,713; U.S. patent application Ser. No. 13/842,859 (US 2014/0068797 A1); U.S. Pat. Nos. 9,260,723; 9,023,649; 9,834,791; 9,637,739; U.S. patent application Ser. No. 14/683,443 (US 2015/0240261 A1); U.S. patent application Ser. No. 14/743,764 (US 2015/0291961 A1); U.S. Pat. Nos. 9,790,490; 9,688,972; 9,580,701; 9,745,562; 9,816,081; 9,677,090; 9,738,687; U.S. application Ser. No. 15/632,222 (US 2017/0369879 A1); U.S. application Ser. No. 15/631,989; U.S. application Ser. No. 15/632,001; and U.S. Pat. No. 9,896,696, each of which is herein incorporated by reference.

In some embodiments, a Cas effector protein comprises one or more of the following activities: a nickase activity, i.e., the ability to cleave a single strand of a nucleic acid molecule; a double stranded nuclease activity, i.e., the ability to cleave both strands of a double stranded nucleic acid and create a double stranded break; an endonuclease activity; an exonuclease activity; and/or a helicase activity, i.e., the ability to unwind the helical structure of a double stranded nucleic acid.

In one embodiment, the RNA-guided DNA endonuclease for use in a construct, composition and/or gene editing method provided herein is selected from Cas9, Cas12a, Cas12b, Cas12c, Cas12d, Cas12e, Cas13a, Cas13b, Cas13c, Cpf1, and MAD7, or homologs, orthologs or paralogs thereof.

### Promoters and Terminators

A promoter for use in any of the nucleic acid constructs and/or gRNA comprising parts provided herein can be any promoter or portion thereof known in the art. In one embodiment, the promoter is an RNA polymerase III promoter (RNA Pol III or Pol III promoter). A Pol III promoter can refer to a nucleotide sequence that directs the transcription of RNA by RNA polymerase III. A Pol III promoter for use in the nucleic acid constructs of gRNA comprising parts provided herein may include a full-length promoter or a fragment thereof sufficient to drive transcription by RNA polymerase III. The Pol III promoter can be a type I, II or III Pol III promoter known in the art. In some cases, the Pol III is any hybrid Pol III promoter known in the art such as the S. cerevisiae hybrid Pol III promoter described in Schramm, L. and Hernandez, N. (2002) Genes Dev. 16:2593-620. Examples of RNA polymerase III promoters for use herein may include, without limitation, promoters for 5S RNA, U6 snRNA, 7SK, RNase P, the RNA component of the Signal Recognition Particle and snoRNAs. In one embodiment, the promoter is the SNR52 promoter. As used herein, SNR52 refers to a C/D box small nucleolar RNA (snoRNA) involved in methylation of rRNA. As used herein, an SNR52 promoter may refer to a full-length promoter sequence, or a fragment thereof.

A promoter for use in any of the nucleic acid constructs and/or gRNA comprising parts provided herein may further comprise one or more genetic elements that serve to specify the prerequisite conditions for transcriptional initiation. A promoter may be encoded by the endogenous genome of a host cell, or it may be introduced as part of a recombinantly engineered polynucleotide. A promoter sequence may be taken from one host species and used to drive expression of a gene in a host cell of a different species. A promoter sequence may also be artificially designed for a particular mode of expression in a particular species, through random mutation or rational design.

A terminator for use in any of the nucleic acid constructs and/or nucleic acid parts provided herein can be any terminator portion thereof known in the art. In one embodiment, the terminator is an RNA polymerase III terminator (RNA Pol III or Pol III terminator). As used herein, an “RNA polymerase III terminator” refers to any nucleotide sequence that is sufficient to terminate a transcript transcribed by RNA polymerase III. As used herein, and unless specified, an RNA polymerase III terminator may refer to the transcribed RNA sequence itself or the DNA sequence encoding it. Examples of RNA polymerase III terminators can include, without limitation, a string of uridine nucleotides of at least 5-6 bases in length. In one embodiment, the RNA polymerase III terminator is UUUUUUUTUUUUUU.

### Expression Vectors

An expression vector for use in the compositions, kits and methods provided herein can include plasmids, yeast artificial chromosomes, 2 μm plasmids, yeast integrative plasmids, yeast replicative plasmids, shuttle vectors, and episomal plasmids. The expression vector may further comprise any desired elements that control the expression of the nucleic acid(s), as well as any elements that enable the replication and maintenance of the expression vector inside a given host cell. Expression vectors for use herein may comprise elements selected from the group consisting of a centromeric (CEN) sequence, an autonomous replication sequence (ARS), a promoter, an origin of replication, a marker gene (e.g., auxotrophic, antibiotic, or other selectable markers and any combination thereof. In some cases, an expression vector may comprise sequences encoding an RNA polymerase III promoter, a plurality of gRNA-tRNA sequence parts or tRNA sequence-gRNA parts, gRNA only parts, spacer sequence only parts, an RNA polymerase III terminator, and/or an RNA-guided DNA endonuclease protein (e.g., Cas9). As used herein, a “host cell” can refer to a cell that contains the expression vector. In some cases, the expression vector can be an expression vector whose replication is temperature sensitive. In some cases, the expression vector may comprise marker genes that can be used for counterselection.

### Selectable Marker Genes

A selectable marker can often encode a gene product providing a specific type of resistance foreign to a non-transformed host cell. This can be resistance to heavy metals, antibiotics or biocides in general. Prototrophy can also be a useful selectable marker of the non-antibiotic variety. Auxotrophic markers can generate nutritional deficiencies in the host cells, and genes correcting those deficiencies can be used for selection.

There is a wide range of selection markers in use in the art and any or all of these can be applied to the methods, compositions and constructs provided herein. The selectable marker genes for use herein can be auxotrophic markers, prototrophic markers, dominant markers, recessive markers, antibiotic resistance markers, catabolic markers, enzymatic markers, fluorescent markers, luminescent markers, directional markers or combinations thereof. Examples of these include, but are not limited to: amdS (acetamide/fluoroacetamide), ble (belomycin-phleomycin resistance), hyg (hygromycinR), nat (nourseotricin R), pyrG (uracil/5FOA), niaD (nitrate/chlorate), sutB (sulphate/selenate), eGFP (Green Fluorescent Protein) and all the different color variants, aygA (colorimetric marker), meta (methionine/selenate), pyrE (orotate P-ribosyl transferase), trpC (anthranilate synthase), argB (ornithine carbamoyltransferase), bar (phosphinothricin acetyltransferase), mutant acetolactate synthase (sulfonylurea resistance), and neomycin phosphotransferase (aminoglycoside resistance). A directional marker gene can be selected from an acetamidase (amdS) gene or a nitrate reductase gene (niaD).

The antibiotic selection markers genes for use in any method, construct or compositions provided herein can be any antibiotic selection marker genes known in the art. The antibiotic selection marker genes used in any of the vectors (e.g., plasmids) utilized in the methods, constructs and compositions provided herein can be chosen based on the microbial host cell. For example, for prokaryotic host cells, the antibiotic selection marker gene can be any genes known in the art that confers resistance against ampicillin, kanamycin, tetracycline, chloramphenicol, zeocin, spectinomycin/streptomycin. For eukaryotic host cells, the antibiotic selection marker gene can be any genes known in the art that confers resistance against bleomycin, phleomycin geneticin, neomycin, hygromycin, puromycin, blasticidin, zeocin.

The auxotrophic selection markers genes can be any auxotrophic selection marker genes known in the art for a particular microbial host cell. The auxotrophic selection marker genes used in any of the plasmids utilized in the methods provided herein for prokaryotic cells can be selected from known amino acid auxotrophic markers. The auxotrophic selection marker genes used in any of the plasmids utilized in the methods, constructs and compositions provided herein for eukaryotic cells can be selected from yeast URA3, LYS2, LEU2, TRP1, HIS3, MET15 and ADE2 or homologs or orthologs thereof.

As described throughout this disclosure, the vectors (e.g., plasmids) utilized in the methods provided herein can further comprise counter-selectable or counterselection marker genes. The counter-selectable marker genes can be genes often also referred to as “death genes” which express toxic gene products that kill producer cells. The counter-selectable marker genes for use in the methods and compositions provided herein can be any ‘death genes’ known in the art. In one embodiment, the counter-selectable or counterselection marker genes are antibiotic, chemical, or temperature-sensitive selection marker genes. The counter-selectable marker genes used in any of the plasmids utilized in the methods provided herein can be chosen based on the microbial host cell. For example, for prokaryotic host cells (e.g., E. coli or C. glutamicum), the counter-selectable marker gene can be selected from sacB, rpsL(strA), tetAR, pheS, thyA, gata-1, or ccdB, the function of which is described in (Reyrat et al. 1998 “Counterselectable Markers: Untapped Tools for Bacterial Genetics and Pathogenesis.” Infect Immun. 66(9): 4011-4017). For eukaryotic host cells, the counter-selectable marker genes can be selected from yeast LYS2, TRP1, MET15, URA3, URA4+ and thymidine kinase or homologs or orthologs thereof.

### Host Cells

As provided herein, the nucleic acid constructs comprising gRNAs interspersed with tRNA sequences at regular intervals generated using the compositions and/or methods provided herein can be used to edit or modify a genetic element (e.g., genome, cosmid or plasmid) of a host cell or engineer the host cell via introducing (e.g., transforming or transducing) one or more gRNA-tRNA comprising constructs generated using the methods and/or compositions provided herein into said host cell. The genomic engineering or editing methods can be applicable to any organism where desired traits can be identified in a population of genetic mutants. The organism can be a microorganism or higher eukaryotic organism.

Thus, as used herein, the term “microorganism” should be taken broadly. It includes, but is not limited to, the two prokaryotic domains, Bacteria and Archaea, as well as certain eukaryotic fungi and protists. However, in certain aspects, “higher” eukaryotic organisms such as insects, plants, and animals can be utilized in the methods taught herein.

Suitable host cells include, but are not limited to bacterial cells, algal cells, plant cells, fungal cells, insect cells, and mammalian cells. In one illustrative embodiment, suitable host cells include E. coli (e.g., strain W3110).

Other suitable host organisms of the present disclosure include microorganisms of the genus Corynebacterium. In some embodiments, preferred Corynebacterium strains/species include: C. efficiens, with the deposited type strain being DSM44549, C. glutamicum, with the deposited type strain being ATCC13032, and C. ammoniagenes, with the deposited type strain being ATCC6871. In some embodiments, the preferred host of the present disclosure is C. glutamicum.

Suitable host strains of the genus Corynebacterium, in particular of the species Corynebacterium glutamicum, are in particular the known wild-type strains: Corynebacterium glutamicum ATCC13032, Corynebacterium acetoglutamicum ATCC15806, Corynebacterium acetoacidophilum ATCC13870, Corynebacterium melassecola ATCC17965, Corynebacterium thermoaminogenes FERM BP-1539, Brevibacterium flavum ATCC14067, Brevibacterium lactofermentum ATCC13869, and Brevibacterium divaricatum ATCC14020; and L-amino acid-producing mutants, or strains, prepared therefrom, such as, for example, the L-lysine-producing strains: Corynebacterium glutamicum FERM-P 1709, Brevibacterium flavum FERM-P 1708, Brevibacterium lactofermentum FERM-P 1712, Corynebacterium glutamicum FERM-P 6463, Corynebacterium glutamicum FERM-P 6464, Corynebacterium glutamicum DM58-1, Corynebacterium glutamicum DG52-5, Corynebacterium glutamicum DSM5714, and Corynebacterium glutamicum DSM12866.

The term “Micrococcus glutamicus” has also been in use for C. glutamicum. Some representatives of the species C. efficiens have also been referred to as C. thermoaminogenes in the prior art, such as the strain FERM BP-1539, for example.

In some embodiments, the host cell of the present disclosure is a eukaryotic cell. Suitable eukaryotic host cells include, but are not limited to: fungal cells, algal cells, insect cells, animal cells, and plant cells. Suitable fungal host cells include, but are not limited to: Ascomycota, Basidiomycota, Deuteromycota, Zygomycota, Fungi imperfecti. Certain preferred fungal host cells include yeast cells and filamentous fungal cells. Suitable filamentous fungi host cells include, for example, any filamentous forms of the subdivision Eumycotina and Oomycota. (see, e.g., Hawksworth et al., In Ainsworth and Bisby's Dictionary of The Fungi, 8^{th} edition, 1995, CAB International, University Press, Cambridge, UK, which is incorporated herein by reference). Filamentous fungi are characterized by a vegetative mycelium with a cell wall composed of chitin, cellulose and other complex polysaccharides. The filamentous fungi host cells are morphologically distinct from yeast.

In certain illustrative, but non-limiting embodiments, the filamentous fungal host cell may be a cell of a species of: Achlya, Acremonium, Aspergillus, Aureobasidium, Bjerkandera, Ceriporiopsis, Cephalosporium, Chrysosporium, Cochliobolus, Corynascus, Cryphonectria, Cryptococcus, Coprinus, Coriolus, Diplodia, Endothis, Fusarium, Gibberella, Ghocladium, Humicola, Hypocrea, Myceliophthora (e.g., Myceliophthora thermophila), Mucor, Neurospora, Penicillium, Podospora, Phlebia, Piromyces, Pyricularia, Rhizomucor, Rhizopus, Schizophyllum, Scytandium, Sporotrichum, Talaromyces, Thermoascus, Thielavia, Tramates, Tolypocladium, Trichoderma, Verticillium, Volvariella, or teleomorphs, or anamorphs, and synonyms or taxonomic equivalents thereof.

Suitable yeast host cells include, but are not limited to: Candida, Hansenula, Saccharomyces, Schizosaccharomyces, Pichia, Kluyveromyces, and Yarrowia. In some embodiments, the yeast cell is Hansenula polymorpha, Saccharomyces cerevisiae, Saccaromyces carlsbergensis, Saccharomyces diastaticus, Saccharomyces norbensis, Saccharomyces kluyveri, Schizosaccharomyces pombe, Pichia pastoris, Pichia finlandica, Pichia trehalophila, Pichia kodamae, Pichia membranaefaciens, Pichia opuntiae, Pichia thermotolerans, Pichia salictaria, Pichia quercuum, Pichia pijperi, Pichia stipitis, Pichia methanohca, Pichia angusta, Kluyveromyces lactis, Candida albicans, or Yarrowia lipolytica.

In certain embodiments, the host cell is an algal such as, Chlamydomonas (e.g., C. reinhardtii) and Phormidium (P. sp. ATCC29409).

In other embodiments, the host cell is a prokaryotic cell. Suitable prokaryotic cells include gram positive, gram negative, and gram-variable bacterial cells. The host cell may be a species of, but not limited to: Agrobacterium, Alicyclobacillus, Anabaena, Anacystis, Acinetobacter, Acidothermus, Arthrobacter, Azobacter, Bacillus, Bifidobacterium, Brevibacterium, Butyrivibrio, Buchnera, Campestris, Camplyobacter, Clostridium, Corynebacterium, Chromatium, Coprococcus, Escherichia, Enterococcus, Enterobacter, Erwinia, Fusobacterium, Faecalibacterium, Francisella, Flavobacterium, Geobacillus, Haemophilus, Helicobacter, Klebsiella, Lactobacillus, Lactococcus, Ilyobacter, Micrococcus, Microbacterium, Mesorhizobium, Methylobacterium, Methylobacterium, Mycobacterium, Neisseria, Pantoea, Pseudomonas, Prochlorococcus, Rhodobacter, Rhodopseudomonas, Rhodopseudomonas, Roseburia, Rhodospirillum, Rhodococcus, Scenedesmus, Streptomyces, Streptococcus, Synecoccus, Saccharomonospora, Staphylococcus, Serratia, Salmonella, Shigella, Thermoanaerobacterium, Tropheryma, Tularensis, Temecula, Thermosynechococcus, Thermococcus, Ureaplasma, Xanthomonas, Xylella, Yersinia, and Zymomonas. In some embodiments, the host cell is Corynebacterium glutamicum.

In some embodiments, the bacterial host strain is an industrial strain. Numerous bacterial industrial strains are known and suitable in the methods and compositions described herein.

In some embodiments, the bacterial host cell is of the Agrobacterium species (e.g., A. radiobacter, A. rhizogenes, A. rubi), the Arthrobacterspecies (e.g., A. aurescens, A. citreus, A. globformis, A. hydrocarboglutamicus, A. mysorens, A. nicotianae, A. paraffineus, A. protophonniae, A. roseoparaffinus, A. sulfureus, A. ureafaciens), the Bacillus species (e.g., B. thuringiensis, B. anthracis, B. megaterium, B. subtilis, B. lentus, B. circulars, B. pumilus, B. lautus, B. coagulans, B. brevis, B. firmus, B. alkaophius, B. licheniformis, B. clausii, B. stearothermophilus, B. halodurans and B. amyloliquefaciens). In particular embodiments, the host cell will be an industrial Bacillus strain including but not limited to B. subtilis, B. pumilus, B. licheniformis, B. megaterium, B. clausii, B. stearothermophilus and B. amyloliquefaciens. In some embodiments, the host cell will be an industrial Clostridium species (e.g., C. acetobutylicum, C. tetani E88, C. lituseburense, C. saccharobutylicum, C. perfringens, C. beijerinckii). In some embodiments, the host cell will be an industrial Corynebacterium species (e.g., C. glutamicum, C. acetoacidophilum). In some embodiments, the host cell will be an industrial Escherichia species (e.g., E. coli). In some embodiments, the host cell will be an industrial Erwinia species (e.g., E. uredovora, E. carotovora, E. ananas, E. herbicola, E. punctata, E. terreus). In some embodiments, the host cell will be an industrial Pantoea species (e.g., P. citrea, P. agglomerans). In some embodiments, the host cell will be an industrial Pseudomonas species, (e.g., P. putida, P. aeruginosa, P. mevalonii). In some embodiments, the host cell will be an industrial Streptococcus species (e.g., S. equisimiles, S. pyogenes, S. uberis). In some embodiments, the host cell will be an industrial Streptomyces species (e.g., S. ambofaciens, S. achromogenes, S. avennitihs, S. coelicolor, S. aureofaciens, S. aureus, S. fungicidicus, S. griseus, S. lividans). In some embodiments, the host cell will be an industrial Zymomonas species (e.g., Z. mobilis, Z. lipolytica), and the like.

In some embodiments, the host cell will be an industrial Escherichia species (e.g., E. coli).

Suitable host strains of the E. coli species comprise: Enterotoxigenic E. coli (ETEC), Enteropathogenic E. coli (EPEC), Enteroinvasive E. coli (EIEC), Enterohemorrhagic E. coli (EHEC), Uropathogenic E. coli (UPEC), Verotoxin-producing E. coli, E. coli O157:H7, E. coli O104:H4, Escherichia coli O121, Escherichia coli O104:H21, Escherichia coli K1, and Escherichia coli NC101. In some embodiments, the present disclosure teaches genomic engineering of E. coli K12, E. coli B, and E. coli C.

In some embodiments, the host cell can be E. coli strains NCTC 12757, NCTC 12779, NCTC 12790, NCTC 12796, NCTC 12811, ATCC 11229, ATCC 25922, ATCC 8739, DSM 30083, BC 5849, BC 8265, BC 8267, BC 8268, BC 8270, BC 8271, BC 8272, BC 8273, BC 8276, BC 8277, BC 8278, BC 8279, BC 8312, BC 8317, BC 8319, BC 8320, BC 8321, BC 8322, BC 8326, BC 8327, BC 8331, BC 8335, BC 8338, BC 8341, BC 8344, BC 8345, BC 8346, BC 8347, BC 8348, BC 8863, and BC 8864.

In some embodiments, the present disclosure teaches host cells that can be verocytotoxigenic E. coli (VTEC), such as strains BC 4734 (O26:H11), BC 4735 (O157:H−), BC 4736, BC 4737 (n.d.), BC 4738 (O157:H7), BC 4945 (O26:H−), BC 4946 (O157:H7), BC 4947 (O111:H−), BC 4948 (O157:H), BC 4949 (O5), BC 5579 (O157:H7), BC 5580 (O157:H7), BC 5582 (O3:H), BC 5643 (O2:H5), BC 5644 (O128), BC 5645 (O55:H−), BC 5646 (O69:H−), BC 5647 (O101:H9), BC 5648 (O103:H2), BC 5850 (O22:H8), BC 5851 (O55:H−), BC 5852 (O48:H21), BC 5853 (O26:H11), BC 5854 (O157:H7), BC 5855 (O157:H−), BC 5856 (O26:H−), BC 5857 (O103:H2), BC 5858 (O26:H11), BC 7832, BC 7833 (O raw form:H−), BC 7834 (ONT:H−), BC 7835 (O103:H2), BC 7836 (O57:H−), BC 7837 (ONT:H−), BC 7838, BC 7839 (O128:H2), BC 7840 (O157:H−), BC 7841 (O23:H−), BC 7842 (O157:H−), BC 7843, BC 7844 (O157:H−), BC 7845 (O103:H2), BC 7846 (O26:H11), BC 7847 (O145:H−), BC 7848 (O157:H−), BC 7849 (O156:H47), BC 7850, BC 7851 (O157:H−), BC 7852 (O157:H−), BC 7853 (O5:H−), BC 7854 (O157:H7), BC 7855 (O157:H7), BC 7856 (O26:H−), BC 7857, BC 7858, BC 7859 (ONT:H−), BC 7860 (O129:H−), BC 7861, BC 7862 (O103:H2), BC 7863, BC 7864 (O raw form:H−), BC 7865, BC 7866 (O26:H−), BC 7867 (O raw form:H−), BC 7868, BC 7869 (ONT:H−), BC 7870 (O113:H−), BC 7871 (ONT:H−), BC 7872 (ONT:H−), BC 7873, BC 7874 (O raw form:H−), BC 7875 (O157:H−), BC 7876 (O111:H−), BC 7877 (O146:H21), BC 7878 (O145:H−), BC 7879 (O22:H8), BC 7880 (O raw form:H−), BC 7881 (O145:H−), BC 8275 (O157:H7), BC 8318 (O55:K−:H−), BC 8325 (O157:H7), and BC 8332 (ONT), BC 8333.

In some embodiments, the present disclosure teaches host cells that can be enteroinvasive E. coli (EIEC), such as strains BC 8246 (O152:K−:H−), BC 8247 (O124:K(72):H3), BC 8248 (O124), BC 8249 (O112), BC 8250 (O136:K(78):H−), BC 8251 (O124:H−), BC 8252 (O144:K−:H−), BC 8253 (O143:K:H−), BC 8254 (O143), BC 8255 (O112), BC 8256 (O28a.e), BC 8257 (O124:H−), BC 8258 (O143), BC 8259 (O167:K−:H5), BC 8260 (O128a.c.:H35), BC 8261 (O164), BC 8262 (O164:K−:H−), BC 8263 (O164), and BC 8264 (O124).

In some embodiments, the present disclosure teaches host cells that can be enterotoxigenic E. coli (ETEC), such as strains BC 5581 (O78:H11), BC 5583 (O2:K1), BC 8221 (O118), BC 8222 (O148:H−), BC 8223 (O111), BC 8224 (O110:H−), BC 8225 (O148), BC 8226 (O118), BC 8227 (O25:H42), BC 8229 (O6), BC 8231 (O153:H45), BC 8232 (O9), BC 8233 (O148), BC 8234 (O128), BC 8235 (O118), BC 8237 (O111), BC 8238 (O110:H17), BC 8240 (O148), BC 8241 (O6H16), BC 8243 (O153), BC 8244 (O15:H−), BC 8245 (O20), BC 8269 (O125a. c:H−), BC 8313 (O6:H6), BC 8315 (O153:H−), BC 8329, BC 8334 (O118:H12), and BC 8339.

In some embodiments, the present disclosure teaches host cells that can be enteropathogenic E. coli (EPEC), such as strains BC 7567 (O86), BC 7568 (O128), BC 7571 (O114), BC 7572 (O119), BC 7573 (O125), BC 7574 (O124), BC 7576 (O127a), BC 7577 (O126), BC 7578 (O142), BC 7579 (O26), BC 7580 (OK26), BC 7581 (O142), BC 7582 (O55), BC 7583 (O158), BC 7584 (O−), BC 7585 (O−), BC 7586 (O−), BC 8330, BC 8550 (O26), BC 8551 (O55), BC 8552 (O158), BC 8553 (O26), BC 8554 (O158), BC 8555 (O86), BC 8556 (O128), BC 8557 (OK26), BC 8558 (O55), BC 8560 (O158), BC 8561 (O158), BC 8562 (O114), BC 8563 (O86), BC 8564 (O128), BC 8565 (O158), BC 8566 (O158), BC 8567 (O158), BC 8568 (O111), BC 8569 (O128), BC 8570 (O114), BC 8571 (O128), BC 8572 (O128), BC 8573 (O158), BC 8574 (O158), BC 8575 (O158), BC 8576 (O158), BC 8577 (O158), BC 8578 (O158), BC 8581 (O158), BC 8583 (O128), BC 8584 (O158), BC 8585 (O128), BC 8586 (O158), BC 8588 (O26), BC 8589 (O86), BC 8590 (O127), BC 8591 (O128), BC 8592 (O114), BC 8593 (O114), BC 8594 (O114), BC 8595 (O125), BC 8596 (O158), BC 8597 (O26), BC 8598 (O26), BC 8599 (O158), BC 8605 (O158), BC 8606 (O158), BC 8607 (O158), BC 8608 (O128), BC 8609 (O55), BC 8610 (O114), BC 8615 (O158), BC 8616 (O128), BC 8617 (O26), BC 8618 (O86), BC 8619, BC 8620, BC 8621, BC 8622, BC 8623, BC 8624 (O158), and BC 8625 (O158).

In some embodiments, the present disclosure also teaches host cells that can be Shigella organisms, including Shigella flexneri, Shigella dysenteriae, Shigella boydii, and Shigella sonnei.

The present disclosure is also suitable for use with a variety of animal cell types, including mammalian cells, for example, human (including 293, WI38, PER.C6 and Bowes melanoma cells), mouse (including 3T3, NS0, NS1, Sp2/0), hamster (CHO, BHK), monkey (COS, FRhL, Vero), and hybridoma cell lines.

In various embodiments, strains that may be used in the practice of the disclosure including both prokaryotic and eukaryotic strains, are readily accessible to the public from a number of culture collections such as American Type Culture Collection (ATCC), Deutsche Sammlung von Mikroorganismen and Zellkulturen GmbH (DSM), Centraalbureau Voor Schimmelcultures (CBS), and Agricultural Research Service Patent Culture Collection, Northern Regional Research Center (NRRL).

In some embodiments, the methods of the present disclosure are also applicable to multi-cellular organisms. For example, the platform could be used for improving the performance of crops. The organisms can comprise a plurality of plants such as Gramineae, Fetucoideae, Poacoideae, Agrostis, Phleum, Dactylis, Sorgum, Setaria, Zea, Oryza, Triticum, Secale, Avena, Hordeum, Saccharum, Poa, Festuca, Stenotaphrum, Cynodon, Coix, Olyreae, Phareae, Compositae or Leguminosae. For example, the plants can be corn, rice, soybean, cotton, wheat, rye, oats, barley, pea, beans, lentil, peanut, yam bean, cowpeas, velvet beans, clover, alfalfa, lupine, vetch, lotus, sweet clover, wisteria, sweet pea, sorghum, millet, sunflower, canola or the like. Similarly, the organisms can include a plurality of animals such as non-human mammals, fish, insects, or the like.

### Transformation of Host Cells

In some embodiments, the constructs as provided herein and/or generated by the methods of the present disclosure may be introduced into the host cells using any of a variety of techniques, including transformation, transfection, transduction, viral infection, gene guns, or Ti-mediated gene transfer. Particular methods include calcium chloride-mediated transformation, calcium phosphate transfection, DEAE-Dextran mediated transfection, lipofection, or electroporation (Davis, L., Dibner, M., Battey, I., 1986 “Basic Methods in Molecular Biology”). Other methods of transformation include for example, lithium acetate transformation and electroporation See, e.g., Gietz et al., Nucleic Acids Res. 27:69-74 (1992); Ito et al., J. Bacterol. 153:163-168 (1983); and Becker and Guarente, Methods in Enzymology 194:182-187 (1991). In some embodiments, transformed host cells are referred to as recombinant host strains.

Methods for transforming a host cell with a construct provided herein or an expression vector comprising a gRNA-tRNA comprising construct as provided herein may differ depending upon the species of the desired host cell. For example, yeast cells may be transformed by lithium acetate treatment (which may further include carrier DNA and PEG treatment) or electroporation. These methods are included for illustrative purposes and are in no way intended to be limiting or comprehensive. Routine experimentation through means well known in the art may be used to determine whether a particular expression vector or transformation method is suited for a given host cell. Furthermore, reagents and vectors suitable for many different host microorganisms are commercially available and/or well known in the art.

### Automation

In one embodiment, the constructs, compositions and methods provided herein are incorporated into a high-throughput (HTP) method for genetic engineering of a host cell. In another embodiment, the methods provided herein can be a molecular tool that is part of the suite of HTP molecular tool sets described in PCT/US18/36360, PCT/US18/36333 or WO 2017/100377, each of which is herein incorporated by reference, for all purposes, to create HTP genetic design libraries, which are derived from, inter alia, scientific insight and iterative pattern recognition. The constructs, compositions and methods provided herein can be used to generate libraries for use in high-throughput methods such as those described in PCT/US18/36360, PCT/US18/36333 or WO 2017/100377. Examples of libraries that can be generated using the methods provided herein can include but are not limited to libraries of nucleic constructs comprising combinations of gRNAs targeting multiple loci in genomes of host cells. Examples of high-throughput genomic engineering methods that can utilize the compositions and methods provided herein can include, but are not limited to, promoter swapping, terminator (stop) swapping, solubility tag swapping, degradation tag swapping or SNP swapping as described in PCT/US18/36360, PCT/US18/36333 or WO 2017/100377. The high-throughput methods can be automated and/or utilize robotics and liquid handling platforms (e.g., plate robotics platform and liquid handling machines known in the art). The high-throughput methods can utilize multi-well plates such as, for example microtiter plates.

In some embodiments, the automated methods of the disclosure comprise a robotic system. The systems outlined herein are generally directed to the use of 96- or 384-well microtiter plates, but as will be appreciated by those in the art, any number of different plates or configurations may be used. In addition, any or all of the steps outlined herein may be automated; thus, for example, the systems may be completely or partially automated. The robotic systems compatible with the methods and compositions provided herein can be those described in PCT/US18/36360, PCT/US18/36333 or WO 2017/100377.

### Applications

The composition and assembly methods provided herein can be used to construct any desired assembly, such as plasmids, vectors, genes, metabolic pathways, minimal genomes, partial genomes, genomes, chromosomes, extrachromosomal nucleic acids, for example, cytoplasmic organelles, such as mitochondria (animals), and in chloroplasts and plastids (plants), and the like.

The constructs, compositions and assembly methods provided herein can be used to generate libraries of nucleic acid constructs comprising a plurality of guide RNAs (gRNAs) interspersed with tRNA sequences at regular intervals such that said each member or subsets of members of said libraries can possess spacer sequences that are directed to different genomic loci. The libraries can contain 2 or more variants, and said multiple variants, can be screened for members having desired characteristics. Such screening may be done by high throughput methods, which may be robotic/automated as provided herein.

The disclosure also further includes products made by the compositions and assembly methods provided herein, for example, the resulting assembled synthetic genes or genomes (synthetic or naturally occurring) and modified optimized genes and genomes, and the use(s) thereof.

The constructs, compositions and assembly methods provided herein can have a wide variety of applications, permitting, for example, the design of pathways for the synthesis of desired products of interest or optimization of one or more sequences whose gene products play a role in the synthesis or expression of a desired product. The compositions and assembly methods provided herein can also be used to generate optimized sequences of a gene or expression thereof or to combine one or more functional domains or motifs of protein encoded by a gene. The gene can be part of a biochemical or metabolic pathway. The biochemical or metabolic pathway can produce a desired product of interest.

The desired product of interest can be any molecule that can be assembled in a cell culture, eukaryotic or prokaryotic expression system or in a transgenic animal or plant. Thus, the nucleic acid molecules or libraries thereof that result from the assembly constructs provided herein may be employed in a wide variety of contexts to produce desired products of interest. In some cases, the product of interest may be a small molecule, enzyme, peptide, amino acid, organic acid, synthetic compound, fuel, alcohol, etc. For example, the product of interest or biomolecule may be any primary or secondary extracellular metabolite. The primary metabolite may be, inter alia, ethanol, citric acid, lactic acid, glutamic acid, glutamate, lysine, threonine, tryptophan and other amino acids, vitamins, polysaccharides, etc. The secondary metabolite may be, inter alia, an antibiotic compound like penicillin, or an immunosuppressant like cyclosporin A, a plant hormone like gibberellin, a statin drug like lovastatin, a fungicide like griseofulvin, etc. The product of interest or biomolecule may also be any intracellular component produced by a host cell, such as: a microbial enzyme, including catalase, amylase, protease, pectinase, glucose isomerase, cellulase, hemicellulase, lipase, lactase, streptokinase, and many others. The intracellular component may also include recombinant proteins, such as: insulin, hepatitis B vaccine, interferon, granulocyte colony-stimulating factor, streptokinase and others. The product of interest may also refer to a protein of interest

### Pathway Assembly

In one embodiment, the constructs, compositions and methods provided herein are used to assemble a gene or a variant thereof. The gene or variant thereof can encode a protein that is part of a metabolic or biochemical pathway. The variant can be a codon optimized version or mutated version of said gene. The metabolic or biochemical pathway can produce a product of interest as provided herein. In one embodiment, the gene sequence or variant thereof can be present as a a repair fragment as provided herein. The pair of homology arms on a repair fragment as provided herein can serve to facilitate targeting of and insertion into a locus in a genetic element (e.g., genome, plasmid, etc.) within a host cell targeted by a cognate gRNA from a nucleic acid construct produced using the methods provided herein using a gene editing method as provided herein. In some cases, the repair fragment can further comprise sequence of a regulatory or control element that can govern an aspect of the gene or variant thereof or the protein encoded thereby such as the transcription, translation, solubility, or degradation thereof. The regulatory or control element can be a promoter, terminator, solubility tag, degradation tag or degron.

In another embodiment, the constructs, compositions and methods provided herein are used to assemble or combine nucleic acid sequence that encode motifs or domains of a target protein. The nucleic acid sequence encoding a particular motif or domain of a target protein can be with a repair fragment provided herein.

### Kits

Also provided by the present disclosure are kits for practicing the methods for generating nucleic acid constructs or libraries derived therefrom as described above. The kit can comprise a mixture containing all of the reagents necessary for assembling nucleic acid constructs comprising a plurality of guide RNAs (gRNAs) interspersed with tRNA sequences at regular intervals. In certain embodiments, a subject kit may contain: (i) a vector backbone comprising type IIs restriction sites, promoter, gRNA and/or terminator sequences as provided herein, (ii) a plurality of nucleic acid parts (gRNA-tRNA sequence parts alone and in some cases along with either gRNA parts or spacer parts), wherein each nucleic acid part comprises type IIs restriction sites and spacer sequences therebetween and (iii) optionally, any enzymes necessary to facilitate scarless assembly of the nucleic acid parts into the vector backbone to generate a nucleic acid construct comprising a plurality of guide RNAs (gRNAs) interspersed with tRNA sequences at regular intervals. A kit provided herein can further comprises a pool of nucleic acid parts for each slot designed to be present in the vector backbone.

In a separate embodiment, the kits provided herein further comprise a repair fragment or pool of repair fragments such that the repair fragment or each repair fragment from the pool comprises homology arms that can facilitate insertion of a genetic edit present on the repair fragment into a specific locus in the genome of a host cell that is targeted by a gRNA supplied in the kit.

Any of the kits provided herein may also contain other reagents described above and below that may be employed in the method, e.g., type IIs restriction enzymes, buffers, nucleic acid guided DNA nucleases or constructs encoding said nucleases and/or competent cells to receive the plasmids, controls etc., depending on how the method is going to be implemented.

The components of the kit may be combined in one container, or each component may be in its own container. For example, the components of the kit may be combined in a single reaction tube or in one or more different reaction tubes.

In addition to above-mentioned components, the subject kit further includes instructions for using the components of the kit to practice the subject method. The instructions for practicing the subject method are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded.

Compositions, kits and methods for assembling of nucleic acid parts and a plasmid backbone as described herein result in a product that is a plasmid comprising an array of two or more gRNA-tRNA sequence units in a tandem arrangement that can used to edit the genome of host cell using CRISPR mediated homology directed repair.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art.

### Example 1: Validation of tRNAs Used in 2RNA-tRNA Constructs for Use in Modular Assembly of 2RNA-tRNA Arrays

### Objective

CRISPR/Cas systems can be used to create multiplexed genomic edits if multiple guide RNAs (gRNAs) are expressed in a host organism. One-way of introducing multiple gRNAs into a host organism entails building guide gRNA-tRNA arrays by employing a modular Golden Gate cloning strategy to stitch together interchangeable, modular parts, each containing a single gRNA sequence. This modular assembly process can reduce the cost of building constructs for expressing a variable number of gRNAs for multiplexed genome editing and can enable the combinatorial re-use of DNA parts as well as pooled, combinatorial assembly of multiplexed gRNA expression constructs. An example of this process of modular assembly of gRNA-tRNA parts is shown in FIGS. 1A-1C. In order to facilitate this process of modular assembly of gRNA-tRNA, this example describes testing of a set of different tRNAs with variable 3′ termini for the processing of each gRNA from a primary transcript of gRNA-tRNAs with the idea that genetic diversity at the 3′ terminus of tRNAs may enable efficient and modular assembly of the gRNA arrays DNA constructs.

### Materials and Methods

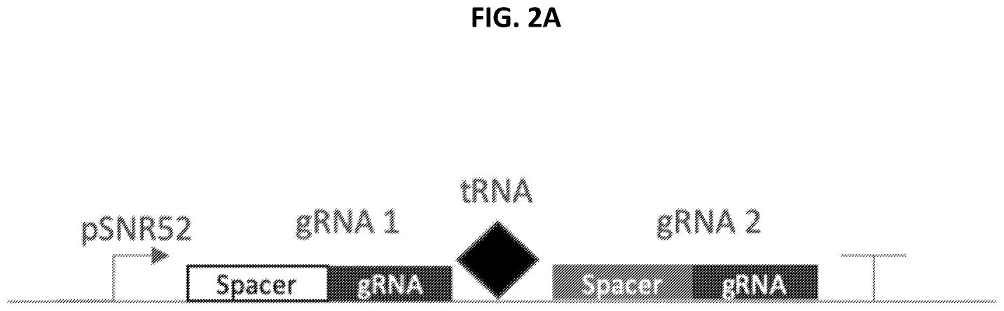

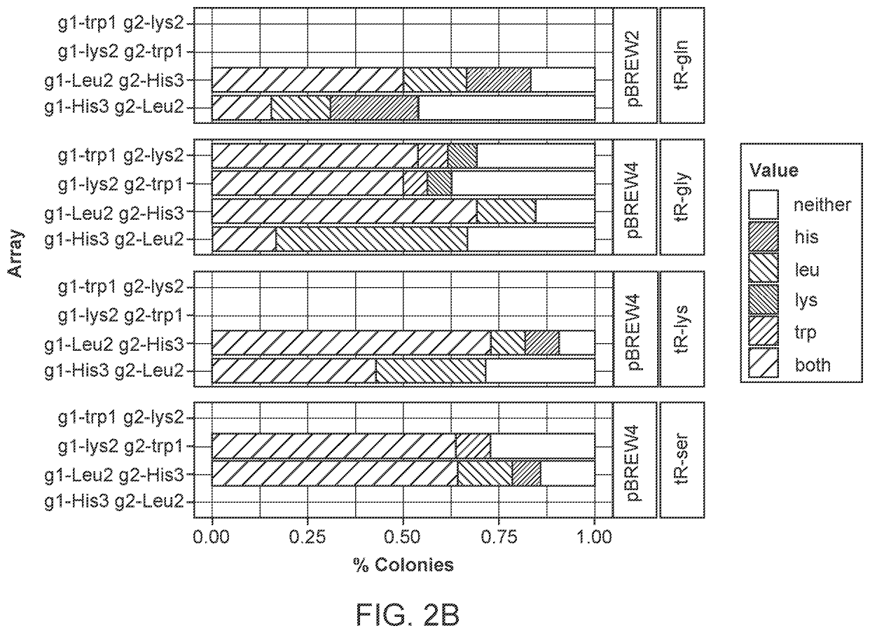

To test tRNAs for their efficacy in initiating gRNA processing in a gRNA-tRNA array, the tRNAs for glycine (tR-gly in FIG. 2B), glutamate (tR-gln in FIG. 2B), lysine (tR-lys in FIG. 2B) and serine (tR-ser in FIG. 2B) were each assembled into a total of sixteen (16) duplex gRNA-tRNAs with spacers as shown in FIG. 2A targeting the loci for the trp1, lys2, leu2 and his3 genes as shown in FIG. 2B. Assembly of the duplex gRNA-tRNAs followed the method depicted in FIGS. 1A-1B except that there were only 2 slots (or gRNA-tRNAs) added into the plasmid backbone and, thus, one set of DNA parts for assembling into slot 1 of the plasmid backbone and a second set of DNA parts for assembling into slot 2 of the plasmid backbone using Golden Gate assembly. The plasmid backbone comprised from 5′ to 3′, a pSNR52 promoter, a BsaI site, a stuffer sequence, a second BsaI site and a gRNA backbone. Additionally, the plasmid backbone comprised sequence for Cas9, S. cerevisiae & E. coli origins of replication, a yeast centromere and selectable markers for E. coli (NptII providing Kanamycin resistance) and S. cerevisiae (pBrew2, KlURA3 providing uracil biosynthesis or pBrew4, NatMX providing resistance to nourseothricin). The DNA parts specific for slot 1 comprised from 5′ to 3′, a BsaI Type IIs restriction endonuclease recognition site, a spacer sequence specific for one of the four (4) genes (i.e., trp1, lys2, leu2 or his3 genes), a gRNA backbone, a tRNA sequence and another BsaI Type IIs restriction endonuclease, while the DNA parts specific for slot 2 comprised from 5′ to 3′, a BsaI Type IIs restriction endonuclease recognition site, a spacer sequence specific for one of the four (4) genes (i.e., trp1, lys2, leu2 or his3 genes) that was not the same or, rather, was different than the gene found in the DNA part specific for slot 1 and a second BsaI restriction endonuclease site. During Golden Gate assembly, digestion of the plasmid backbone and sets of DNA parts for slot 1 and slot 2 with BsaI generated a 5′ overhang in slot 1 DNA parts that was complementary in sequence to a 3′ overhang created in the SNR52 promoter in the plasmid backbone, a 5′ overhang in the slot 2 DNA parts that were complementary in sequence to a 3′ overhang in the slot 1 DNA parts, and a 5′ overhang in the gRNA backbone of the plasmid backbone that was complementary in sequence to a 3′ overhang in the slot 2 DNA parts.

### Results and Conclusions

As shown in FIG. 2B, all four of the tRNAs tested were capable of initiating efficient processing of multiple gRNA transcripts as evidenced by the efficient multiplexed editing that was observed for each gRNA-tRNA duplex. As a result, use of all or any combination of the tested tRNAs can be used DNA parts that can be assembled into gRNA-tRNA arrays in a modular fashion for subsequent use in the multiplexed editing of host organisms.

### Example 2: Use of 2RNA-tRNA Duplex Arrays for Use in Genome Editing Using Multiple Repair Fragments

### Objective

CRISPR/Cas systems can be used to create multiplexed genomic edits if multiple guide RNAs (gRNAs) are expressed in a host organism. One-way of introducing multiple gRNAs into a host organism entails building guide gRNA-tRNA arrays by employing a modular Golden Gate cloning strategy to stitch together interchangeable, modular parts, each containing a single gRNA sequence. This modular assembly process can reduce the cost of building constructs for expressing a variable number of gRNAs for multiplexed genome editing and can enable the combinatorial re-use of DNA parts as well as pooled, combinatorial assembly of multiplexed gRNA expression constructs. An example of this process of modular assembly of gRNA-tRNA parts is shown in FIGS. 1A-1C. This example describes testing the efficacy of gRNA-tRNA arrays in genome editing when used in combination with multiple repair fragments for each targeted genomic locus.

### Materials and Methods

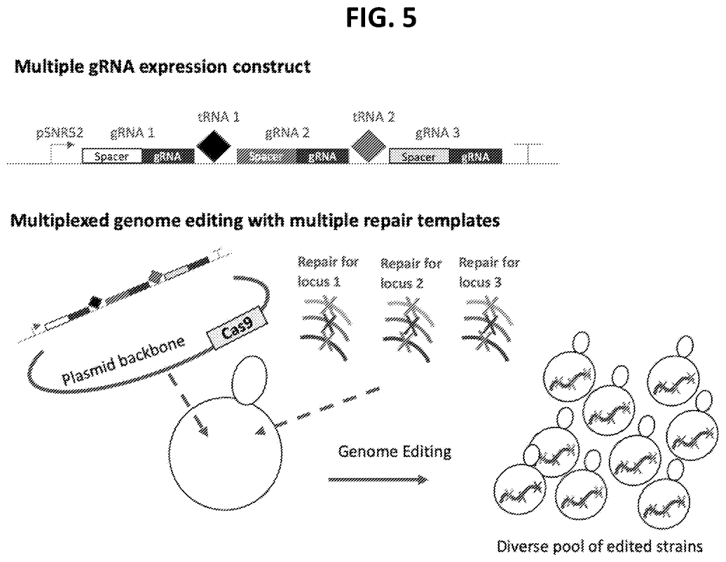

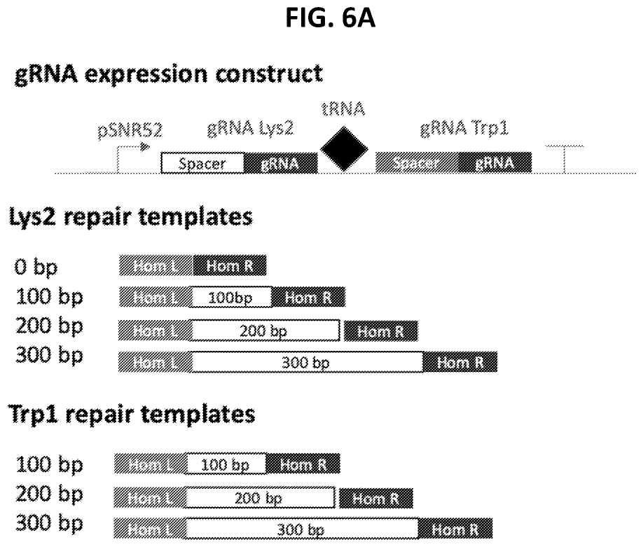

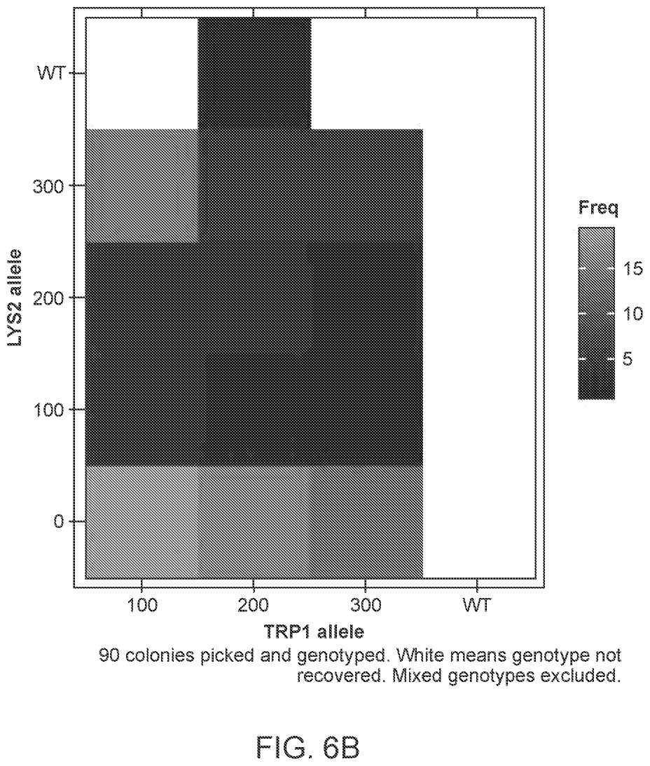

To test the efficacy of gRNA-tRNA arrays for use in genome editing when paired with multiple repair fragments per targeted loci, a method as generally depicted in FIG. 5 was employed. In contrast to FIG. 5, the method employed in this Example utilized a duplex gRNA-tRNA with spacers targeting the loci for the Lys2 and Trp1 genes under the control of the pSNR52 promoter (see FIG. 6A). The duplex gRNA-tRNA was generated as described in Example 1. The plasmid backbone into which the gRNA-tRNA was inserted further comprised sequence for Cas9, S. cerevisiae & E. coli origins of replication, a yeast centromere and selectable markers. NptII for E. coli and NatMX for S. cerevisiae. As shown in FIG. 5, the multiple gRNA containing plasmid was transformed into S. cerevisiae along with multiple repair templates for each locus (i.e., Lys1 and Trp1 genes) targeted by the gRNA. The specific repair fragments tested in this Example for the Lys1 and Trp1 loci are shown in FIG. 6A. More specifically, Lys2 and Trp1 were simultaneously edited with 4 and 3 repair templates, respectively, for integrating payloads of varied length. Following transformation, growth and subsequent isolation of transformants, said isolated transformants were genotyped by PCR to determine the insertion size in each colony.

### Results and Conclusions

As shown in FIG. 6B, genotyping of multiplexed pooled repair fragment colonies showed that all possible combinations of edited alleles were recovered by the experiments described in this Example. Accordingly, gRNA-tRNA arrays generated using the methods provided throughout this disclosure are effective for use in genome editing with multiple repair fragment pools per targeted loci.

### Example 3: Use of 2RNA-tRNA Arrays Assembled Using Modular Assembly Method Described Herein in Multiplexed Genome Editing

### Objective

CRISPR/Cas systems can be used to create multiplexed genomic edits if multiple guide RNAs (gRNAs) are expressed in a host organism. One-way of introducing multiple gRNAs into a host organism entails building guide gRNA-tRNA arrays by employing a modular Golden Gate cloning strategy to stitch together interchangeable, modular parts, each containing a single gRNA sequence. This modular assembly process can reduce the cost of building constructs for expressing a variable number of gRNAs for multiplexed genome editing and can enable the combinatorial re-use of DNA parts as well as pooled, combinatorial assembly of multiplexed gRNA expression constructs. An example of this process of modular assembly of gRNA-tRNA parts is shown in FIGS. 1A-1C. The goal or objective of this Example is to provide a proof-of-concept that gRNA-tRNA arrays assembled using the modular assembly method described herein are effective in multiplexed genome editing using repair fragments. gRNA-tRNA arrays were assembled and then PCR amplified for transformation as linear DNA fragments for gap repair following transformation or transformed as circular gRNA expression plasmids.

### Materials and Methods

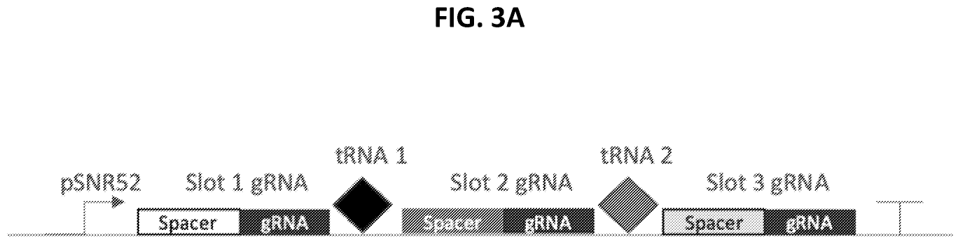

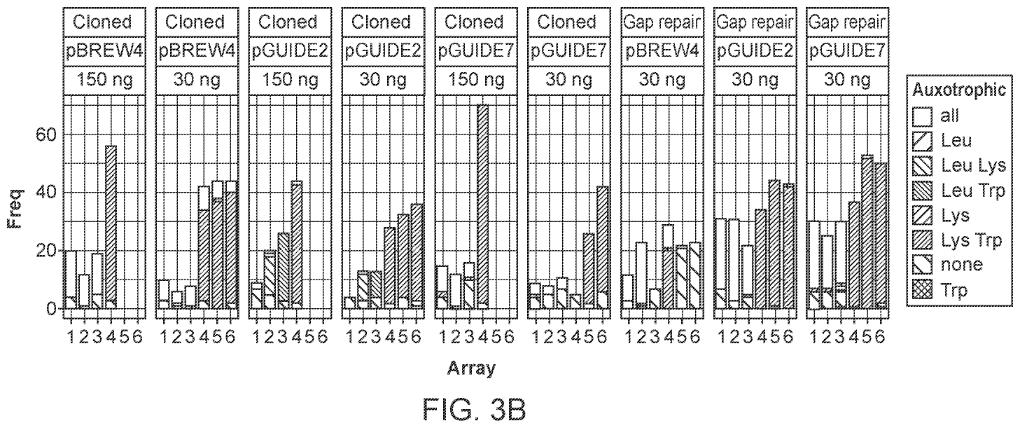

In order to test the use of gRNA-tRNA arrays generated using the modular assembly methods described herein in genome editing, six (6) triplex gRNA-tRNA arrays described in Table 1, each containing the structure shown in FIG. 3A were built using a plasmid backbone as shown in FIG. 1A and varied gRNA and tRNA parts as described in Table 1 using the method described in FIG. 1B. The plasmid backbone into which each of the six (6) gRNA-tRNA arrays were assembled (either prior to transformation (“cloned” in FIG. 3B) or in vivo (“gap repair” in FIG. 3B)) also contained a selectable marker (e.g., URA3, KanMX, etc.), and machinery necessary for maintenance within a host cell (i.e., centromere and autonomously replicating sequence, CEN-ARS). Subsequently, each of the six (6) triplex gRNA-tRNA array containing plasmids was transformed into a Saccharomyces cerevisiae host cell along with a plasmid comprising a Cas9 nuclease gene (i.e., pBREW4 in FIG. 3B) or a Saccharomyces cerevisiae host cell comprising a Cas9 nuclease gene integrated into the genome of the cell (i.e., pGuide2 & pGuide7 in FIG. 3B). In parallel, linear DNA molecules acting as repair templates for the targeted loci (i.e., Leu2 gene, Lys2 gene and/or Trp1 gene) containing the desired edits were included in the transformation. Following transformation recovered strains were plated on solid media selective for the plasmid backbone (YPD+100 μg/mL Nourseothricin). Colonies were then cultured and tested for presence of the desired edits.

TABLE 1
gRNA-tRNA array configurations tested as shown in FIGS 3A-3B.

| | slot 1 | | slot 2 | | slot 3 |
|---|---|---|---|---|---|
| array: | gRNA | tRNA 1 | gRNA | tRNA 2 | gRNA |
| triplex-1 | Leu2 | tR-Lys | Lys2 | tR-gly | Trp1 |
| triplex-2 | Leu2 | tR-Ser | Lys2 | tR-Lys | Trp1 |
| triplex-3 | Leu2 | tR-gly | Lys2 | tR-Ser | Trp1 |
| triplex-4 | NT | tR-Lys | Lys2 | tR-gly | Trp1 |
| triplex-5 | NT | tR-Ser | Lys2 | tR-Lys | Trp1 |
| triplex-6 | NT | tR-gly | Lys2 | tR-Ser | Trp1 |

NT = non-targeting guide

### Results and Conclusions

As shown in FIG. 3B, efficient triplex editing of Leu2, Lys2 and Trp1 was observed using arrays of gRNAs and tRNAs. Lysine, serine and glycine tRNAs support efficient gRNA expression and multiplexed genome editing with both plasmid-based (pBrew4) and integrated (pGuide2 & pGuide7) Cas9 expression. As a result, multiplexed gRNA-tRNAs generated using the modular assembly method described in FIGS. 1A-1C was effective in promoting genome editing of Cas9 expressing host cells.

### Numbered Embodiments of the Disclosure

Other subject matter contemplated by the present disclosure is set out in the following numbered embodiments:

1. A nucleic acid construct comprising, from 5′ to 3′, a first type IIs restriction site, a guide RNA (gRNA) and a tRNA sequence with a second type IIs restriction site within the tRNA sequence, wherein the gRNA comprises a spacer sequence comprising sequence complementary to a target sequence present at a locus in a genetic element in a cell.

2. The nucleic acid construct of embodiment 1, wherein the tRNA sequence comprises one or more of the group consisting of a pretRNA acceptor stem, a D-loop arm and a TψC-loop arm.

3. The nucleic acid construct of embodiment 1 or 2, wherein the tRNA sequence comprises an active site for one or more of the group consisting of RNase P, RNase Z, RNase E, RNase F and RNase D.

4. The nucleic acid construct of any one of the above embodiments, wherein the tRNA sequence comprises an entire pre-tRNA sequence.

5. The nucleic acid construct of embodiment 4, wherein the pre-tRNA sequence is selected from tRNA-ser, tRNA-gln, tRNA-lys or tRNA-gly.

6. The nucleic acid construct of any one of the above embodiments, wherein the second type IIs restriction site is within the 3′ terminus of the tRNA sequence.

7. The nucleic acid construct of any one of the above embodiments, wherein the first and/or second type IIs restriction site is BsaI.

8. The nucleic acid construct of any one of the above embodiments, wherein the gRNA is a single guide RNA (sgRNA).

9. The nucleic acid construct of any one of the above embodiments, wherein the cell is a prokaryotic cell or eukaryotic cell.

10. A composition comprising:
(a) a plasmid backbone comprising, from 5′ to 3′, a promoter sequence, a first type IIs restriction site, a stuffer sequence and a second type IIs restriction site; and
(b) a plurality of gRNA-tRNA sequence parts, wherein each gRNA-tRNA sequence part in the plurality, comprises, from 5′ to 3′, a first type IIs restriction site, a guide RNA (gRNA) and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality, wherein the first and second type IIs restriction sites in the plasmid backbone allow for insertion of each gRNA-tRNA sequence part of the plurality of gRNA-tRNA sequence parts of (b) into the plasmid backbone upon cleavage.

11. The composition of embodiment 10, wherein the promoter sequence is a Pol III promoter.

12. The composition of embodiment 10 or 11, wherein the promoter sequence is a pSNR52 promoter.

13. The composition of any one of embodiments 10-12, wherein the second type IIs restriction site in each gRNA-tRNA sequence part from the plurality is within the 3′ terminus of the tRNA sequence.

14. The composition of any one of embodiments 10-13, wherein the tRNA sequence in each gRNA-tRNA sequence part from the plurality comprises one or more of the group consisting of a pretRNA acceptor stem, a D-loop arm and a TψC-loop arm.

15. The composition of any one of embodiments 10-14, wherein the tRNA sequence in each gRNA-tRNA sequence part from the plurality comprises an active site for one or more of the group consisting of RNase P, RNase Z, RNase E, RNase F and RNase D.

16. The composition of any one of embodiments 10-15, wherein the tRNA sequence in each gRNA-tRNA sequence part from the plurality comprises a full pre-tRNA sequence.

17. The composition of embodiment 16, wherein the pre-tRNA sequence is selected from tRNA-ser, tRNA-gln, tRNA-lys or tRNA-gly.

18. The composition of any one of embodiments 10-17, further comprising a type IIs restriction enzyme that recognizes the first and/or the second type IIs restriction sites in the plasmid backbone and each gRNA-tRNA sequence part from the plurality.

19. The composition of embodiment 18, wherein the type IIs restriction enzyme is BsaI.

20. The composition of any one of embodiments 10-19, wherein the gRNA in each gRNA-tRNA sequence part from the plurality is a single guide RNA (sgRNA).

21. The composition of embodiment 20, wherein the spacer sequence in each gRNA-tRNA sequence part from the plurality of gRNA-tRNA sequence parts comprises sequence complementary to a target sequence present at a different locus in a genetic element in a host cell than the spacer sequence in each other gRNA-tRNA sequence part from the plurality.

22. The composition of any one of embodiments 10-21, wherein the plasmid backbone further comprises a scaffold sequence 3′ to the second type IIs restriction site, wherein the scaffold sequence comprises sequence necessary to bind to an RNA-guided DNA endonuclease.

23. The composition of embodiment 22, wherein one of the gRNA-tRNA sequence parts from the plurality differs from each other gRNA-tRNA sequence part from the plurality in that said gRNA-tRNA sequence part comprises, from 5′ to 3′, the first type IIs restriction site, a spacer sequence and the second type IIS restriction site.

24. The composition of any one of embodiments 10-23, wherein each gRNA-tRNA sequence part from the plurality of gRNA-tRNA sequence parts is represented by a pool of gRNA-tRNA sequence parts.

25. The composition of embodiment 24, wherein each gRNA-tRNA sequence part within a pool comprises a spacer sequence that comprises sequence complementary to a target sequence present at an identical locus in a genetic element in a host cell as the spacer sequence in each other gRNA-tRNA sequence part within the pool.

26. The composition of embodiment 24, wherein each gRNA-tRNA sequence part within a pool comprises a spacer sequence that comprises sequence complementary to a target sequence present at a different locus in a genetic element in a host cell as the spacer sequence in each other gRNA-tRNA sequence part within the pool.

27. The composition of any one of embodiments 24-26, wherein each gRNA-tRNA sequence part within a pool comprises a spacer sequence that comprises sequence complementary to a target sequence present at a different locus in a genetic element in a host cell than the spacer sequence in each gRNA-tRNA sequence part from each other pool.

28. A method for preparing a plasmid comprising an array of guide RNA (gRNA)-tRNA sequences in tandem arrangement, the method comprising:
(a) incubating under conditions that allow for digestion, a mixture comprising:
   (i) a type IIs restriction enzyme,
   (ii) a plasmid backbone comprising, from 5′ to 3′, a promoter sequence, a first type IIs restriction site, a stuffer sequence and a second Type IIs restriction site, wherein the first and the second type IIs restriction site are recognized and digested by the type IIs restriction enzyme of (i), and
   (iii) a plurality of gRNA-tRNA sequence parts, wherein each gRNA-tRNA sequence part in the plurality, comprises, from 5′ to 3′, a first type IIs restriction site, a gRNA and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality, and wherein the first and the second type IIs restriction site in each gRNA-tRNA sequence part in the plurality are recognized and digested by the type IIs restriction enzyme of (i),
wherein digestion of the plasmid backbone of (i) removes the stuffer sequence and generates a first end within the promoter sequence and a second end distal to the first end, while digestion of each gRNA-tRNA sequence part within the plurality generates opposing first and second ends on each gRNA-tRNA sequence part that are different than the opposing first and second ends on each other gRNA-tRNA sequence part in the plurality, and wherein digestion of one gRNA-tRNA sequence part from the plurality generates a first end comprising sequence complementary to the first end of the plasmid backbone, while digestion for each other gRNA-tRNA sequence part from the plurality generates a first end that comprises sequence complementary to the second end of a gRNA-tRNA cleavage sequence part from each other gRNA-tRNA sequence part, and wherein digestion of one gRNA-tRNA sequence part from the plurality generates a second end comprising sequence complementary to the second end of the plasmid backbone; and
(b) incubating the mixture with a ligase under conditions that allow for hybridization and covalent joining of first and/or second ends that comprise complementary sequence present within the mixture, wherein ligation operably links the promoter sequence in the plasmid backbone to an array of gRNA-tRNA sequence parts from the plurality of gRNA-tRNA sequence parts in tandem arrangement, thereby generating an assembled plasmid comprising an array of gRNA-tRNA sequences in tandem arrangement.

29. The method of embodiment 28, wherein the promoter sequence is a Pol III promoter.

30. The method of embodiment 28 or 29, wherein the promoter sequence is a pSNR52 promoter.

31. The method of any one of embodiments 28-30, wherein the second type IIs restriction site in each gRNA-tRNA sequence part from the plurality is within the 3′ terminus of the tRNA sequence.

32. The method of any one of embodiments 28-31, wherein the tRNA sequence in each gRNA-tRNA sequence part from the plurality comprises one or more of the group consisting of a pretRNA acceptor stem, a D-loop arm and a TψC-loop arm.

33. The method of any one of embodiments 28-32, wherein the tRNA sequence in each gRNA-tRNA sequence part from the plurality comprises an active site for one or more of the group consisting of RNase P, RNase Z, RNase E, RNase F and RNase D.

34. The method of any one of embodiments 28-33, wherein the tRNA sequence in each gRNA-tRNA sequence part from the plurality comprises a full pre-tRNA sequence.

35. The method of embodiment 34, wherein the pre-tRNA sequence is selected from tRNA-ser, tRNA-gln, tRNA-lys or tRNA-gly.

36. The method of any one of embodiments 28-35, wherein the type IIs restriction enzyme is BsaI.

37. The method of any one of embodiments 28-36, wherein the gRNA in each gRNA-tRNA sequence part from the plurality is a single guide RNA (sgRNA).

38. The method of embodiment 37, wherein the spacer sequence in each gRNA-tRNA sequence part from the plurality of gRNA-tRNA sequence parts comprises sequence complementary to a target sequence present at a different locus in a genetic element in a host cell than the spacer sequence in each other gRNA-tRNA sequence part from the plurality.

39. The method of any one of embodiments 28-38, wherein the plasmid backbone further comprises a scaffold comprising sequence necessary to bind to an RNA-guided DNA endonuclease 3′ to the second type IIs restriction site.

40. The method of embodiment 39, wherein the one gRNA-tRNA sequence part from the plurality whose second end comprises sequence complementary to the second end of the plasmid backbone following digestion with the type IIS restriction enzyme of (i) differs from each other gRNA-tRNA sequence part from the plurality in that said gRNA-tRNA sequence part comprises, from 5′ to 3′, the first type IIS restriction site, a spacer sequence and the second type IIs restriction site.

41. The method of any one of embodiments 28-40, wherein each gRNA-tRNA sequence part from the plurality of gRNA-tRNA sequence parts is represented by a pool of gRNA-tRNA sequence parts, thereby generating a library of assembled plasmids comprising an array of gRNA-tRNA sequences in tandem arrangement.

42. The method of embodiment 41, wherein each gRNA-tRNA sequence part within a pool comprises a spacer sequence that comprises sequence complementary to a target sequence present at an identical locus in a genetic element in a host cell as the spacer sequence in each other gRNA-tRNA sequence part within the pool.

43. The method of embodiment 41, wherein each gRNA-tRNA sequence part within a pool comprises a spacer sequence that comprises sequence complementary to a target sequence present at a different locus in a genetic element in a host cell as the spacer sequence in each other gRNA-tRNA sequence part within the pool.

44. The method of any one of embodiments 41-43, wherein each gRNA-tRNA sequence part within a pool comprises a spacer sequence that comprises sequence complementary to a target sequence present at a different locus in a genetic element in a host cell than the spacer sequence in each gRNA-tRNA sequence part from each other pool.

45. The method of any one of embodiments 28-40, further comprising (c) propagating the assembled plasmid comprising an array of gRNA-tRNA sequences in tandem arrangement in a microbial cell; and (d) isolating nucleic acid from the microbial cell of step (c), wherein the isolated nucleic acid comprises the assembled plasmid.

46. The method of any one of embodiments 41-44, further comprising (c) propagating each of the assembled plasmids comprising an array of gRNA-tRNA sequences in tandem arrangement from the library in a microbial cell; and (d) isolating nucleic acid from each of the microbial cells of step (c), wherein the isolated nucleic acid from each of the microbial cells comprises an assembled plasmid from the library.

47. The method of embodiment 45 or 46, wherein the propagating of step (c) entails transforming the microbial cell and growing in growth media.

48. The method of any one of embodiments 45-47, wherein the microbial cell is E. coli.

49. The method of embodiment 47 or 48, wherein step (d) comprises picking transformants from step (c).

50. The method of embodiment 49, further comprising (e) sequencing nucleic acid isolated from the transformants from step (d).

51. A method for editing the genome of a host cell, the method comprising:
(a) introducing an assembled plasmid comprising an array of guide RNA (gRNA)-tRNA sequences in tandem arrangement generated using the method of any one of embodiments 28-50 into a host cell, wherein the host cell expresses an RNA-guided DNA endonuclease or an RNA-guided DNA endonuclease is introduced into the host cell along with the assembled plasmid and wherein the host cell utilizes the tRNA sequence in each gRNA-tRNA sequence in the array to release each gRNA from the array of gRNA-tRNA sequences in tandem arrangement; and
(b) introducing a plurality of repair fragments under conditions that allow for homology-directed repair (HDR) utilizing the RNA-guided DNA endonuclease, wherein the plurality of repair fragments comprises a repair fragment for each gRNA released in step (a) that comprises homology arms on opposing ends of the repair fragment that comprise sequence complementary to the locus targeted by the gRNA and at least one genetic edit, thereby editing the genome of the host cell.

52. The method of embodiment 51, wherein the at least one genetic edit is selected from the group consisting of a substitution, an inversion, an insertion, a deletion, a single nucleotide polymorphism, and any combination thereof.

53. The method of embodiment 51 or 52, wherein each repair fragment from the plurality of repair fragments is provided on a plasmid or as a linear fragment.

54. The method of any one of embodiments 51-53, wherein each repair fragment from the plurality of repair fragments is provided as a single-stranded DNA (ssDNA) or double-stranded DNA (dsDNA) linear fragment.

55. The method of any one of embodiments 51-54, wherein each repair fragment from the plurality of repair fragments is represented by a pool of repair fragments.

56. The method of embodiment 55, wherein the at least one genetic edit within each repair fragment in the pool of repair fragment is different than the at least one genetic edit in each other repair fragment in the pool of repair fragments.

57. The method of any one of embodiments 51-56, wherein the assembled plasmid comprising an array of gRNA-tRNA sequences in tandem arrangement further comprises a selectable marker gene.

58. The method of any one of embodiments 51-57, wherein the assembled plasmid comprising an array of gRNA-tRNA sequences in tandem arrangement further comprises a centromere and autonomously replicating sequence.

59. The method of any one of embodiments 51-58, wherein the RNA-guided DNA endonuclease is selected from Cas9, Cas12a, Cas12b, Cas12c, Cas12d, Cas12e, Cas13a, Cas13b, Cas13c, Cpf1, and MAD7, or homologs, orthologs or paralogs thereof.

60. The method of any one of embodiments 51-59, wherein the RNA-guided DNA endonuclease is encoded on a plasmid, encoded in the genome of the host cell, translated from RNA, or introduced into the host cell as protein.

61. The method of any one of embodiments 51-60, wherein the host cell is a eukaryotic cell.

62. The method of embodiment 61, wherein the host cell is a yeast cell.

63. The method of embodiment 62, wherein the yeast cell is Saccharomyces cere mac.

64. The method of embodiment 61, wherein the host cell is a filamentous fungus.

65. The method of embodiment 64, wherein the filamentous fungus is Aspergillus niger.

66. The method of any one of embodiments 51-60, wherein the host cell is a prokaryotic cell.

67. The method of embodiment 66, wherein the prokaryotic host cell is Escherichia coli or Corynebacterium glutamicum.

68. A nucleic acid construct comprising two or more guide RNA (gRNA)-tRNA sequence units in tandem arrangement, wherein the tRNA sequence in each unit is different than the tRNA sequence in an adjacent unit, and wherein each gRNA in each gRNA-tRNA unit comprises a spacer sequence comprising sequence complementary to a locus in a genetic element in a host cell.

69. The nucleic acid construct of embodiment 68, wherein the tRNA sequence in each gRNA-tRNA sequence unit comprises one or more of the group consisting of a pretRNA acceptor stem, a D-loop arm and a TψC-loop arm.

70. The nucleic acid construct of embodiment 68 or 69, wherein the tRNA sequence in each gRNA-tRNA sequence unit comprises an active site for one or more of the group consisting of RNase P, RNase Z, RNase E, RNase F and RNase D.

71. The nucleic acid construct of any one of embodiments 68-70, wherein the tRNA sequence in each gRNA-tRNA sequence part from the plurality comprises a full pre-tRNA sequence.

72. The nucleic acid construct of embodiment 71, wherein the pre-tRNA sequence is selected from tRNA-ser, tRNA-gln, tRNA-lys or tRNA-gly.

73. The nucleic acid construct of any one of embodiments 68-72, wherein the gRNA in each gRNA-tRNA sequence unit is a single guide RNA (sgRNA).

74. The nucleic acid construct of any one of embodiments 68-73, wherein the spacer sequence in each gRNA-tRNA sequence unit comprises sequence complementary to a target sequence present at a different locus in a genetic element in a host cell than the spacer sequence in each other gRNA-tRNA sequence unit.

75. The nucleic acid construct of any one of embodiments 68-73, wherein the spacer sequence in each gRNA-tRNA sequence unit comprises sequence complementary to a target sequence present at an identical locus in a genetic element in a host cell as the spacer sequence in each other gRNA-tRNA sequence unit.

76. The nucleic acid construct of any one of embodiments 68-75, further comprising a promoter sequence that is operably linked to the two or more gRNA-tRNA sequence units in tandem arrangement.

77. The nucleic acid construct of any one of embodiments 68-76, further comprising a terminator sequence that is operably linked to the two or more gRNA-tRNA sequence units in tandem arrangement.

78. The nucleic acid construct of any one of embodiments 68-75, wherein each of the two or more gRNA-tRNA sequence units comprises a promoter sequence and a terminator sequence operably linked thereto.

79. The nucleic acid construct of any one of embodiments 76-78, wherein the promoter sequence is a Pol III promoter.

80. The nucleic acid construct of any one of embodiments 76-79, wherein the terminator sequence is a Pol III terminator.

81. An expression cassette comprising the nucleic acid construct of any one of embodiments 68-80.

82. A vector comprising the expression cassette of embodiment 81.

83. A host cell comprising the nucleic acid construct of any one of embodiments 68-80.

84. A genetically modified cell comprising a genetic edit, the cell having been edited by the introduction of the construct of any one of embodiments 68-80.

1. A nucleic acid construct comprising, from 5′ to 3′, a first type IIs restriction site, a guide RNA (gRNA) and a tRNA sequence with a second type IIs restriction site within the tRNA sequence, wherein the gRNA comprises a spacer sequence comprising sequence complementary to a target sequence present at a locus in a genetic element in a cell.

2.-3. (canceled)

4. The nucleic acid construct of claim 1, wherein the tRNA sequence comprises an entire pre-tRNA sequence, wherein the full pre-tRNA sequence is selected from tRNA-ser, tRNA-gln, tRNA-lys or tRNA-gly.

5. (canceled)

6. The nucleic acid construct of claim 1, wherein the second type IIs restriction site is within the 3′ terminus of the tRNA sequence.

7.-9. (canceled)

10. A composition comprising:
(a) a plasmid backbone comprising, from 5′ to 3′, a promoter sequence, a first type IIs restriction site, a stuffer sequence and a second type IIs restriction site; and
(b) a plurality of gRNA-tRNA sequence parts, wherein each gRNA-tRNA sequence part in the plurality, comprises, from 5′ to 3′, a first type IIs restriction site, a guide RNA (gRNA) and a tRNA sequence with a second type IIs restriction site within the tRNA sequence, wherein the gRNA comprises a spacer sequence comprising sequence complementary to a target sequence present at a locus in a genetic element in a host cell, wherein the tRNA sequences in each gRNA-tRNA sequence part in the plurality is different than the tRNA sequence in each other gRNA-tRNA sequence part in the plurality,
wherein the first and second type IIs restriction sites in the plasmid backbone allow for insertion of each gRNA-tRNA sequence part of the plurality of gRNA-tRNA sequence parts of (b) into the plasmid backbone upon cleavage.

11.-12. (canceled)

13. The composition of claim 10, wherein the second type IIs restriction site in each gRNA-tRNA sequence part from the plurality is within the 3′ terminus of the tRNA sequence.

14.-15. (canceled)

16. The composition of claim 10, wherein the tRNA sequence in each gRNA-tRNA sequence part from the plurality comprises a full pre-tRNA sequence, wherein the full pre-tRNA sequence is selected from tRNA-ser, tRNA-gln, tRNA-lys or tRNA-gly.

17. (canceled)

18. The composition of claim 10, further comprising a type IIs restriction enzyme that recognizes the first and/or the second type IIs restriction sites in the plasmid backbone and each gRNA-tRNA sequence part from the plurality.

19.-20. (canceled)

21. The composition of claim 10, wherein the spacer sequence in each gRNA-tRNA sequence part from the plurality of gRNA-tRNA sequence parts comprises sequence complementary to a target sequence present at a different locus in a genetic element in a host cell than the spacer sequence in each other gRNA-tRNA sequence part from the plurality.

22. The composition of claim 10, wherein the plasmid backbone further comprises a scaffold sequence 3′ to the second type IIs restriction site, wherein the scaffold sequence comprises sequence necessary to bind to an RNA-guided DNA endonuclease.

23. The composition of claim 22, wherein one of the gRNA-tRNA sequence parts from the plurality differs from each other gRNA-tRNA sequence part from the plurality in that said gRNA-tRNA sequence part comprises, from 5′ to 3′, the first type IIs restriction site, a spacer sequence and the second type IIS restriction site.

24. The composition of claim 10, wherein each gRNA-tRNA sequence part from the plurality of gRNA-tRNA sequence parts is represented by a pool of gRNA-tRNA sequence parts.

25. The composition of claim 24, wherein each gRNA-tRNA sequence part within a pool comprises a spacer sequence that comprises sequence complementary to a target sequence present at either a different locus or an identical locus in a genetic element in a host cell as the spacer sequence in each other gRNA-tRNA sequence part within the pool.

26. (canceled)

27. The composition of claim 24, wherein each gRNA-tRNA sequence part within a pool comprises a spacer sequence that comprises sequence complementary to a target sequence present at a different locus in a genetic element in a host cell than the spacer sequence in each gRNA-tRNA sequence part from each other pool.

28.-50. (canceled)

51. A method for editing the genome of a host cell, the method comprising:
(a) introducing an assembled plasmid comprising the nucleic acid construct of claim 68 into a host cell, wherein the host cell expresses an RNA-guided DNA endonuclease or an RNA-guided DNA endonuclease is introduced into the host cell along with the assembled plasmid and wherein the host cell utilizes the tRNA sequence in each gRNA-tRNA sequence in the nucleic acid construct to release each gRNA from the nucleic acid construct; and
(b) introducing a plurality of repair fragments under conditions that allow for homology-directed repair (HDR) utilizing the RNA-guided DNA endonuclease, wherein the plurality of repair fragments comprises a repair fragment for each gRNA released in step (a) that comprises homology arms on opposing ends of the repair fragment that comprise sequence complementary to the locus targeted by the gRNA and at least one genetic edit, thereby editing the genome of the host cell.

52.-54. (canceled)

55. The method of claim 51, wherein each repair fragment from the plurality of repair fragments is represented by a pool of repair fragments.

56.-67. (canceled)

68. A nucleic acid construct comprising two or more guide RNA (gRNA)-tRNA sequence units in tandem arrangement, wherein the tRNA sequence in each unit is different than the tRNA sequence in an adjacent unit, and wherein each gRNA in each gRNA-tRNA unit comprises a spacer sequence comprising sequence complementary to a locus in a genetic element in a host cell.

69.-70. (canceled)

71. The nucleic acid construct of claim 68, wherein the tRNA sequence in each gRNA-tRNA sequence part from the plurality comprises a full pre-tRNA sequence, wherein the pre-tRNA sequence is selected from tRNA-ser, tRNA-gln, tRNA-lys or tRNA-gly.

72.-73. (canceled)

74. The nucleic acid construct of claim 68, wherein the spacer sequence in each gRNA-tRNA sequence unit comprises sequence complementary to a target sequence present at a different locus or an identical locus in a genetic element in a host cell than the spacer sequence in each other gRNA-tRNA sequence unit.

75. (canceled)

76. The nucleic acid construct of claim 68, further comprising a promoter sequence and/or a terminator sequence that is operably linked to the two or more gRNA-tRNA sequence units in tandem arrangement.

77. (canceled)

78. The nucleic acid construct of claim 68, wherein each of the two or more gRNA-tRNA sequence units comprises a promoter sequence and a terminator sequence operably linked thereto.

79.-84. (canceled)
